SEQUENCE LISTING (390 sequences)

SEQ ID NO: 1-9 - skipped (9 entries)

SEQ ID NO: 10 - protein, 8 aa, Artificial Sequence
  anti-CD123 HCDR1
GFTFSSYG

SEQ ID NO: 11 - protein, 8 aa, Artificial Sequence
  anti-CD123 HCDR2
ISGSGGST

SEQ ID NO: 12 - protein, 17 aa, Artificial Sequence
  anti-CD123 HCDR3
AKEKLRYFDWLSDAFDI

SEQ ID NO: 13 - protein, 12 aa, Artificial Sequence
  anti-CD123 LCDR1
HSVLYSSNNKNY

SEQ ID NO: 14 - protein, 3 aa, Artificial Sequence
  anti-CD123 LCDR2
WAS

SEQ ID NO: 15 - protein, 10 aa, Artificial Sequence
  anti-CD123 LCDR3
QQYYSTPPTT

SEQ ID NO: 16 - protein, 114 aa, Artificial Sequence
  anti-CD123 VH
DIVMTQSPDSLAVSLGERATINCKSSHSVLYSSNNKNYLAWYQQKPGQPPKLLIYWASTRESGVPDRFSGSGSGTDFTLTISSLQAEDVAVYYCQQYYSTPPTTFGGGTKVEIK

SEQ ID NO: 17 - protein, 124 aa, Artificial Sequence
  anti-CD123 VL
EVQLLESGGGLVQPGGSLRLSCAASGFTFSSYGMSWVRQAPGKGLEGVSAISGSGGSTYYADSVKGRFTISRDNSKNTLYLQMNSLRAEDTAVYYCAKEKLRYFDWLSDAFDIWGQGTMVTVSS

SEQ ID NO: 18 - protein, 258 aa, Artificial Sequence
  anti-CD123 scFv
DIVMTQSPDSLAVSLGERATINCKSSHSVLYSSNNKNYLAWYQQKPGQPPKLLIYWASTRESGVPDRFSGSGSGTDFTLTISSLQAEDVAVYYCQQYYSTPPTTFGGGTKVEIKGGGGSGGGGSGGGGSGGGGSEVQLLESGGGLVQPGGSLRLSCAASGFTFSSYGMSWVRQAPGKGLEGVSAISGSGGSTYYADSVKGRFTISRDNSKNTLYLQMNSLRAEDTAVYYCAKEKLRYFDWLSDAFDIWGQGTMVTVSS

SEQ ID NO: 19 - protein, 8 aa, Artificial Sequence
  anti-CD3epsilon HCDR1
GYTFSRST

SEQ ID NO: 20 - protein, 8 aa, Artificial Sequence
  anti-CD3epsilon HCDR2
INPSSAYT

SEQ ID NO: 21 - protein, 14 aa, Artificial Sequence
  anti-CD3epsilon HCDR3
ARPQVHYDYNGFPY

SEQ ID NO: 22 - protein, 7 aa, Artificial Sequence
  anti-CD3epsilon LCDR1
ASSSVSY

SEQ ID NO: 23 - protein, 3 aa, Artificial Sequence
  anti-CD3epsilon LCDR2
DSS

SEQ ID NO: 24 - protein, 9 aa, Artificial Sequence
  anti-CD3epsilon LCDR3
QQWSRNPPT

SEQ ID NO: 25 - protein, 121 aa, Artificial Sequence
  anti-CD3epsilon VH
QVQLVQSGPEVKKPGSSVKVSCKASGYTFSRSTMHWVRQAPGQGLEWIGYINPSSAYTNYNQKFKDRVTITADKSTSTAYMELSSLRSEDTAVYYCARPQVHYDYNGFPYWGQGTLVTVSS

SEQ ID NO: 26 - protein, 108 aa, Artificial Sequence
  anti-CD3epsilon VL
DIQMTQSPSTLSASVGDRVTMTCSASSSVSYMNWYQQKPGKAPKRWIYDSSKLASGVPSRFSGSGSGTEYTLTISSLQPDDFATYYCQQWSRNPPTFGGGTKVEIKRS

SEQ ID NO: 27 - protein, 249 aa, Artificial Sequence
  anti-CD3epsilon scFv
QVQLVQSGPEVKKPGSSVKVSCKASGYTFSRSTMHWVRQAPGQGLEWIGYINPSSAYTNYNQKFKDRVTITADKSTSTAYMELSSLRSEDTAVYYCARPQVHYDYNGFPYWGQGTLVTVSSGGGGSGGGGSGGGGSGGGGSDIQMTQSPSTLSASVGDRVTMTCSASSSVSYMNWYQQKPGKAPKRWIYDSSKLASGVPSRFSGSGSGTDYTLTISSLQPDDFATYYCQQWSRNPPTFGGGTKVEIKRS

SEQ ID NO: 28-30 - skipped (3 entries)

SEQ ID NO: 31 - protein, 777 aa, Artificial Sequence
  anti-CD123 x anti-CD3epsilon bispecific construct amino acid sequence
MEAPAQLLFLLLLWLPDTTGDIVMTQSPDSLAVSLGERATINCKSSHSVLYSSNNKNYLAWYQQKPGQPPKLLIYWASTRESGVPDRFSGSGSGTDFTLTISSLQAEDVAVYYCQQYYSTPPTTFGGGTKVEIKGGGGSGGGGSGGGGSGGGGSEVQLLESGGGLVQPGGSLRLSCAASGFTFSSYGMSWVRQAPGKGLEGVSAISGSGGSTYYADSVKGRFTISRDNSKNTLYLQMNSLRAEDTAVYYCAKEKLRYFDWLSDAFDIWGQGTMVTVSSSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGSGGGGSPSQVQLVQSGPEVKKPGSSVKVSCKASGYTFSRSTMHWVRQAPGQGLEWIGYINPSSAYTNYNQKFKDRVTITADKSTSTAYMELSSLRSEDTAVYYCARPQVHYDYNGFPYWGQGTLVTVSSGGGGSGGGGSGGGGSGGGGSDIQMTQSPSTLSASVGDRVTMTCSASSSVSYMNWYQQKPGKAPKRWIYDSSKLASGVPSRFSGSGSGTDYTLTISSLQPDDFATYYCQQWSRNPPTFGGGTKVEIKRS

SEQ ID NO: 32 - DNA, 699 bp, Artificial Sequence
  modified immunoglobulin constant region
tcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggt

SEQ ID NO: 33 - protein, 233 aa, Artificial Sequence
  modified immunoglobulin constant region
SSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 34 - DNA, 693 bp, Artificial Sequence
  modified immunoglobulin constant region
gagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggt

SEQ ID NO: 35 - protein, 231 aa, Artificial Sequence
  modified immunoglobulin constant region
EPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 36 - protein, 15 aa, Artificial Sequence
  immunoglobulin hinge region
EPKSSDKTHTSPPSS

SEQ ID NO: 37 - protein, 15 aa, Artificial Sequence
  immunoglobulin hinge region
EPKSCDKTHTSPPCS

SEQ ID NO: 38 - protein, 15 aa, Artificial Sequence
  immunoglobulin hinge region
EPKSSDKTHTSPPCS

SEQ ID NO: 39 - protein, 15 aa, Artificial Sequence
  immunoglobulin hinge region
EPKSSDKTHTCPPCS

SEQ ID NO: 40 - protein, 15 aa, Artificial Sequence
  immunoglobulin hinge region
EPKSCDKTHTSPPSS

SEQ ID NO: 41 - protein, 15 aa, Artificial Sequence
  immunoglobulin hinge region
EPKSSDKTHTCPPSS

SEQ ID NO: 42 - protein, 15 aa, Artificial Sequence
  immunoglobulin hinge region
EPKSCDKTHTSPPCS

SEQ ID NO: 43 - protein, 15 aa, Artificial Sequence
  immunoglobulin hinge region
EPKSCDKTHTSPPCP

SEQ ID NO: 44 - protein, 15 aa, Artificial Sequence
  immunoglobulin hinge region
EPKSSDKTHTSPPSP

SEQ ID NO: 45 - protein, 15 aa, Artificial Sequence
  immunoglobulin hinge region
EPKSCDKTHTSPPCP

SEQ ID NO: 46 - protein, 15 aa, Artificial Sequence
  immunoglobulin hinge region
EPKSSDKTHTSPPCP

SEQ ID NO: 47 - protein, 15 aa, Artificial Sequence
  immunoglobulin hinge region
EPKSSDKTHTCPPCP

SEQ ID NO: 48 - protein, 15 aa, Artificial Sequence
  immunoglobulin hinge region
EPKSCDKTHTSPPSP

SEQ ID NO: 49 - protein, 15 aa, Artificial Sequence
  immunoglobulin hinge region
EPKSSDKTHTCPPSP

SEQ ID NO: 50 - protein, 6 aa, Artificial Sequence
  Fc binding domain linker
SCPPCP

SEQ ID NO: 51 - protein, 20 aa, Artificial Sequence
  Fc binding domain linker
NYGGGGSGGGGSGGGGSGNS

SEQ ID NO: 52 - protein, 38 aa, Artificial Sequence
  Fc binding domain linker
NYGGGGSGGGGSGGGGSGNYGGGGSGGGGSGGGGSGNS

SEQ ID NO: 53 - protein, 8 aa, Artificial Sequence
  Fc binding domain linker
GGGGSGNS

SEQ ID NO: 54 - protein, 10 aa, Artificial Sequence
  Fc binding domain linker
NYGGGGSGNS

SEQ ID NO: 55 - protein, 13 aa, Artificial Sequence
  Fc binding domain linker
GGGGSGGGGSGNS

SEQ ID NO: 56 - protein, 15 aa, Artificial Sequence
  Fc binding domain linker
NYGGGGSGGGGSGNS

SEQ ID NO: 57 - protein, 18 aa, Artificial Sequence
  Fc binding domain linker
GGGGSGGGGSGGGGSGNS

SEQ ID NO: 58 - protein, 8 aa, Artificial Sequence
  Fc binding domain linker
GCPPCPNS

SEQ ID NO: 59 - protein, 15 aa, Artificial Sequence
  Fc binding domain linker
GGGGSGGGGSGGGGS

SEQ ID NO: 60 - protein, 16 aa, Artificial Sequence
  Fc binding domain linker
SGGGGSGGGGSGGGGS

SEQ ID NO: 61 - protein, 20 aa, Artificial Sequence
  Fc binding domain linker
GGGGSGGGGSGGGGSGGGGS

SEQ ID NO: 62 - protein, 25 aa, Artificial Sequence
  Fc binding domain linker
GGGGSGGGGSGGGGSGGGGSGGGGS

SEQ ID NO: 63 - protein, 21 aa, Artificial Sequence
  Fc binding domain linker
QRHNNSSLNTGTQMAGHSPNS

SEQ ID NO: 64 - protein, 16 aa, Artificial Sequence
  Fc binding domain linker
SSLNTGTQMAGHSPNS

SEQ ID NO: 65 - protein, 18 aa, Artificial Sequence
  Fc binding domain linker
QRHNNSSLNTGTQMAGHS

SEQ ID NO: 66 - protein, 14 aa, Artificial Sequence
  Fc binding domain linker
EVQIPLTESYSPNS

SEQ ID NO: 67 - protein, 20 aa, Artificial Sequence
  Fc binding domain linker
NSLANQEVQIPLTESYSPNS

SEQ ID NO: 68 - protein, 19 aa, Artificial Sequence
  Fc binding domain linker
SGGGGSGGGGSGGGGSPNS

SEQ ID NO: 69 - protein, 19 aa, Artificial Sequence
  Fc binding domain linker
SGGGGSGGGGSGGGGSPGS

SEQ ID NO: 70 - protein, 17 aa, Artificial Sequence
  Fc binding domain linker
GGGGSGGGGSGGGGSPS

SEQ ID NO: 71-109 - skipped (39 entries)

SEQ ID NO: 110 - protein, 14 aa, Artificial Sequence
  anti-CD3 LCDR1
GSSTGAVTSGYYPN

SEQ ID NO: 111 - protein, 14 aa, Artificial Sequence
  anti-CD3 LCDR1
RSSTGAVTSGYYPN

SEQ ID NO: 112 - protein, 14 aa, Artificial Sequence
  anti-CD3 LCDR1
GSSTGAVTSGNYPN

SEQ ID NO: 113 - protein, 7 aa, Artificial Sequence
  anti-CD3 LCDR2
GTKFLAP

SEQ ID NO: 114 - protein, 7 aa, Artificial Sequence
  anti-CD3 LCDR2
ATDMRPS

SEQ ID NO: 115 - protein, 7 aa, Artificial Sequence
  anti-CD3 LCDR2
GTKFLAP

SEQ ID NO: 116 - protein, 9 aa, Artificial Sequence
  anti-CD3 LCDR3
ALWYSNRWV

SEQ ID NO: 117 - protein, 9 aa, Artificial Sequence
  anti-CD3 LCDR3
ALWYSNRWV

SEQ ID NO: 118 - protein, 9 aa, Artificial Sequence
  anti-CD3 LCDR3
VLWYSNRWV

SEQ ID NO: 119 - protein, 5 aa, Artificial Sequence
  anti-CD3 HCDR1
IYAMN

SEQ ID NO: 120 - protein, 5 aa, Artificial Sequence
  anti-CD3 HCDR1
KYAMN

SEQ ID NO: 121 - protein, 5 aa, Artificial Sequence
  anti-CD3 HCDR1
SYAMN

SEQ ID NO: 122 - protein, 19 aa, Artificial Sequence
  anti-CD3 HCDR2
RIRSKYNNYATYYADSVKS

SEQ ID NO: 123 - protein, 19 aa, Artificial Sequence
  anti-CD3 HCDR2
RIRSKYNNYATYYADSVKD

SEQ ID NO: 124 - protein, 19 aa, Artificial Sequence
  anti-CD3 HCDR2
RIRSKYNNYATYYADSVKG

SEQ ID NO: 125 - protein, 14 aa, Artificial Sequence
  anti-CD3 HCDR3
HGNFGNSYVSFFAY

SEQ ID NO: 126 - protein, 14 aa, Artificial Sequence
  anti-CD3 HCDR3
HGNFGNSYISYWAY

SEQ ID NO: 127 - protein, 14 aa, Artificial Sequence
  anti-CD3 HCDR3
HGNFGNSYLSFWAY

SEQ ID NO: 128 - protein, 5 aa, Artificial Sequence
  peptide linker
GGGGS

SEQ ID NO: 129 - protein, 25 aa, Artificial Sequence
  peptide linker
  MISC_FEATURE (6)..(10): may be present or absent
  MISC_FEATURE (11)..(15): may be present or absent
  MISC_FEATURE (16)..(20): may be present or absent
  MISC_FEATURE (21)..(25): may be present or absent
GGGGSGGGGSGGGGSGGGGSGGGGS

SEQ ID NO: 130 - skipped

SEQ ID NO: 131 - protein, 216 aa, Artificial Sequence
  Modified immunoglobulin constant region
APEAAGAPSVFLFPPKPKDTLMISRTPEVTCVVVDVSHEDPEVKFNWYVDGVEVHNAKTKPREEQYNSTYRVVSVLTVLHQDWLNGKAYACAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 132 - protein, 11 aa, Artificial Sequence
  Fc binding domain linker
SSLNTGTQPNS

SEQ ID NO: 133 - DNA, 342 bp, Artificial Sequence
  OMT1 variable light chain domain
gacatcgtgatgacccagtctccagactccctggctgtgtctctgggcgagagggccaccatcaactgcaagtccagccacagtgttttatacagctccaacaataagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatagtactcctccgaccactttcggcggagggaccaaggtggagatcaaa

SEQ ID NO: 134 - protein, 114 aa, Artificial Sequence; identical to SEQ ID NO: 16
  OMT1 variable light chain domain

SEQ ID NO: 135 - DNA, 372 bp, Artificial Sequence
  OMT1 variable heavy chain domain
gaggtgcagctgttggagtctgggggaggcttggtacagcctggggggtccctgagactctcctgtgcagcctctggattcacctttagcagctatggcatgagctgggtccgccaggctccagggaaggggctggagggggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagaaaagttacgatattttgactggttatccgatgcttttgatatctggggccaagggacaatggtcaccgtctcttca

SEQ ID NO: 136 - protein, 124 aa, Artificial Sequence; identical to SEQ ID NO: 17
  OMT1 variable heavy chain domain

SEQ ID NO: 137 - DNA, 36 bp, Artificial Sequence
  OMT1 CDRL1
cacagtgttttatacagctccaacaataagaactac

SEQ ID NO: 138 - protein, 12 aa, Artificial Sequence
  OMT1 CDRL1
HSVLYSSNNKNY

SEQ ID NO: 139 - DNA, 9 bp, Artificial Sequence
  OMT1 CDRL2
tgggcatct

SEQ ID NO: 140 - protein, 3 aa, Artificial Sequence
  OMT1 CDRL2
WAS

SEQ ID NO: 141 - DNA, 30 bp, Artificial Sequence
  OMT1 CDRL3
cagcaatattatagtactcctccgaccact

SEQ ID NO: 142 - protein, 10 aa, Artificial Sequence
  OMT1 CDRL3
QQYYSTPPTT

SEQ ID NO: 143 - DNA, 24 bp, Artificial Sequence
  OMT1 CDRH1
ggattcacctttagcagctatggc

SEQ ID NO: 144 - protein, 8 aa, Artificial Sequence
  OMT1 CDRH1
GFTFSSYG

SEQ ID NO: 145 - DNA, 24 bp, Artificial Sequence
  OMT1 CDRH2
attagtggtagtggtggtagcaca

SEQ ID NO: 146 - protein, 8 aa, Artificial Sequence
  OMT1 CDRH2
ISGSGGST

SEQ ID NO: 147 - DNA, 51 bp, Artificial Sequence
  OMT1 CDRH3
gcgaaagaaaagttacgatattttgactggttatccgatgcttttgatatc

SEQ ID NO: 148 - protein, 17 aa, Artificial Sequence
  OMT1 CDRH3
AKEKLRYFDWLSDAFDI

SEQ ID NO: 149-308 - skipped (160 entries)

SEQ ID NO: 309 - DNA, 2340 bp, Artificial Sequence
  humanized bispecific construct TRI129
atggaagcaccagcgcagcttctcttcctcctgctactctggctcccagataccaccggtgaggtgcagctgttggagtctgggggaggcttggtacagcctggggggtccctgagactctcctgtgcagcctctggattcacctttagcagctatggcatgagctgggtccgccaggctccagggaaggggctggagggggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagaaaagttacgatattttgactggttatccgatgcttttgatatctggggccaagggacaatggtcaccgtctcttcaggtggaggcggttcaggcggaggtggatccggcggtggcggctccggtggcggcggatctgacatcgtgatgacccagtctccagactccctggctgtgtctctgggcgagagggccaccatcaactgcaagtccagccacagtgttttatacagctccaacaataagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatagtactcctccgaccactttcggcggagggaccaaggtggagatcaaatcctcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggttccggaggagggggttcaggtgggggaggttctggcggcgggggaagcccttcacaggtgcaactggtgcagagtggacccgaggttaaaaaaccagggtcctccgttaaggttagctgcaaagcctctggctacacattttccaggagtacaatgcactgggtgaggcaggctcctggacagggactcgagtggatcgggtatatcaacccatctagcgcctataccaattacaaccaaaagtttaaggaccgagttaccattaccgctgacaaatccaccagtacagcttatatggagctgtcatctcttaggtccgaggacactgctgtttattactgcgctcgtcctcaggttcactatgactataatggttttccctactggggtcagggaaccctggtgactgtctcttctggcggtggaggcagcggtgggggtgggtctggaggcggtggcagtggcggcggaggctctgatattcagatgactcagtctcctagcactctcagcgccagcgtgggggatcgtgtgacaatgacttgctccgctagcagtagtgtgtcttacatgaattggtatcagcagaagcccgggaaagcacctaagcgctggatctatgactcttccaagctggcaagtggtgtcccctcacggttctctggctcaggttctggtactgactatactttgactatctcctccctccagcccgatgatttcgctacctattattgtcagcagtggagccgtaacccacccactttcggaggcggtaccaaagtggagatcaagaggtcataa

SEQ ID NO: 310 - protein, 759 aa, Artificial Sequence
  humanized bispecific construct TRI129
EVQLLESGGGLVQPGGSLRLSCAASGFTFSSYGMSWVRQAPGKGLEGVSAISGSGGSTYYADSVKGRFTISRDNSKNTLYLQMNSLRAEDTAVYYCAKEKLRYFDWLSDAFDIWGQGTMVTVSSGGGGSGGGGSGGGGSGGGGSDIVMTQSPDSLAVSLGERATINCKSSHSVLYSSNNKNYLAWYQQKPGQPPKLLIYWASTRESGVPDRFSGSGSGTDFTLTISSLQAEDVAVYYCQQYYSTPPTTFGGGTKVEIKSSSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGSGGGGSPSQVQLVQSGPEVKKPGSSVKVSCKASGYTFSRSTMHWVRQAPGQGLEWIGYINPSSAYTNYNQKFKDRVTITADKSTSTAYMELSSLRSEDTAVYYCARPQVHYDYNGFPYWGQGTLVTVSSGGGGSGGGGSGGGGSGGGGSDIQMTQSPSTLSASVGDRVTMTCSASSSVSYMNWYQQKPGKAPKRWIYDSSKLASGVPSRFSGSGSGTDYTLTISSLQPDDFATYYCQQWSRNPPTFGGGTKVEIKRS

SEQ ID NO: 311 - DNA, 2334 bp, Artificial Sequence
  humanized bispecific construct TRI130
atggaagcaccagcgcagcttctcttcctcctgctactctggctcccagataccaccggtgacatcgtgatgacccagtctccagactccctggctgtgtctctgggcgagagggccaccatcaactgcaagtccagccacagtgttttatacagctccaacaataagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatagtactcctccgaccactttcggcggagggaccaaggtggagatcaaaggtggaggcggttcaggcggaggtggatccggcggtggcggctccggtggcggcggatctgaggtgcagctgttggagtctgggggaggcttggtacagcctggggggtccctgagactctcctgtgcagcctctggattcacctttagcagctatggcatgagctgggtccgccaggctccagggaaggggctggagggggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagaaaagttacgatattttgactggttatccgatgcttttgatatctggggccaagggacaatggtcaccgtctcctcgagtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggaatacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggttccggaggagggggttcaggtgggggaggttctggcggcgggggaagcccttcacaggtgcaactggtgcagagtggacccgaggttaaaaaaccagggtcctccgttaaggttagctgcaaagcctctggctacacattttccaggagtacaatgcactgggtgaggcaggctcctggacagggactcgagtggatcgggtatatcaacccatctagcgcctataccaattacaaccaaaagtttaaggaccgagttaccattaccgctgacaaatccaccagtacagcttatatggagctgtcatctcttaggtccgaggacactgctgtttattactgcgctcgtcctcaggttcactatgactataatggttttccctactggggtcagggaaccctggtgactgtctcttctggcggtggaggcagcggtgggggtgggtctggaggcggtggcagtggcggcggaggctctgatattcagatgactcagtctcctagcactctcagcgccagcgtgggggatcgtgtgacaatgacttgctccgctagcagtagtgtgtcttacatgaattggtatcagcagaagcccgggaaagcacctaagcgctggatctatgactcttccaagctggcaagtggtgtcccctcacggttctctggctcaggttctggtactgactatactttgactatctcctccctccagcccgatgatttcgctacctattattgtcagcagtggagccgtaacccacccactttcggaggcggtaccaaagtggagatcaagaggtcataa

SEQ ID NO: 312 - protein, 757 aa, Artificial Sequence
  humanized bispecific construct TRI130
DIVMTQSPDSLAVSLGERATINCKSSHSVLYSSNNKNYLAWYQQKPGQPPKLLIYWASTRESGVPDRFSGSGSGTDFTLTISSLQAEDVAVYYCQQYYSTPPTTFGGGTKVEIKGGGGSGGGGSGGGGSGGGGSEVQLLESGGGLVQPGGSLRLSCAASGFTFSSYGMSWVRQAPGKGLEGVSAISGSGGSTYYADSVKGRFTISRDNSKNTLYLQMNSLRAEDTAVYYCAKEKLRYFDWLSDAFDIWGQGTMVTVSSSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGSGGGGSPSQVQLVQSGPEVKKPGSSVKVSCKASGYTFSRSTMHWVRQAPGQGLEWIGYINPSSAYTNYNQKFKDRVTITADKSTSTAYMELSSLRSEDTAVYYCARPQVHYDYNGFPYWGQGTLVTVSSGGGGSGGGGSGGGGSGGGGSDIQMTQSPSTLSASVGDRVTMTCSASSSVSYMNWYQQKPGKAPKRWIYDSSKLASGVPSRFSGSGSGTDYTLTISSLQPDDFATYYCQQWSRNPPTFGGGTKVEIKRS

SEQ ID NO: 313-336 - skipped (24 entries)

SEQ ID NO: 337 - protein, 777 aa, Artificial Sequence; identical to SEQ ID NO: 31
  humanized bispecific construct TRI130

SEQ ID NO: 338-340 - skipped (3 entries)

SEQ ID NO: 341 - protein, 107 aa, Artificial Sequence
  Cris-7 variable light chain sequence
QVVLTQSPAIMSAFPGEKVTMTCSASSSVSYMNWYQQKSGTSPKRWIYDSSKLASGVPARFSGSGSGTSYSLTISSMETEDAATYYCQQWSRNPPTFGGGTKLQITR

SEQ ID NO: 342 - protein, 121 aa, Artificial Sequence
  Cris-7 variable heavy chain sequence
QVQLQQSGAELARPGASVKMSCKASGYTFTRSTMHWVKQRPGQGLEWIGYINPSSAYTNYNQKFKDKATLTADKSSSTAYMQLSSLTSEDSAVYYCASPQVHYDYNGFPYWGQGTLVTVSA

SEQ ID NO: 343 - protein, 106 aa, Artificial Sequence [sequence omitted]

SEQ ID NO: 344 - protein, 120 aa, Artificial Sequence [sequence omitted]

SEQ ID NO: 345 - protein, 5 aa, Artificial Sequence
  Cris7 and DRA222 VH CDR1 (Kabat)
RSTMH

SEQ ID NO: 346 - protein, 16 aa, Artificial Sequence
  Cris7 and DRA222 VH CDR2 (Kabat)
YINPSSAYTNYNQKFK

SEQ ID NO: 347 - protein, 11 aa, Artificial Sequence
  Cris7 and DRA222 VH CDR3 (Kabat)
QVHYDYNGFPY

SEQ ID NO: 348 - protein, 10 aa, Artificial Sequence
  Cris7 and DRA222 VL CDR1 (Kabat)
SASSSVSYMN

SEQ ID NO: 349 - protein, 7 aa, Artificial Sequence
  Cris7 and DRA222 VL CDR2 (Kabat)
DSSKLAS

SEQ ID NO: 350 - protein, 9 aa, Artificial Sequence
  Cris7 and DRA222 VL CDR3 (Kabat)
QQWSRNPPT

SEQ ID NO: 351 - protein, 8 aa, Artificial Sequence
  Cris7 and DRA222 VH CDR1 (IMGT)
GYTFTRST

SEQ ID NO: 352 - protein, 8 aa, Artificial Sequence
  Cris7 and DRA222 VH CDR2 (IMGT)
INPSSAYT

SEQ ID NO: 353 - protein, 9 aa, Artificial Sequence
  Cris7 and DRA222 VH CDR3 (IMGT)
QQWSRNPPT

SEQ ID NO: 354 - protein, 7 aa, Artificial Sequence
  Cris7 and DRA222 VL CDR1 (IMGT)
ASSSVSY

SEQ ID NO: 355 - protein, 3 aa, Artificial Sequence
  Cris7 and DRA222 VL CDR2 (IMGT)
DSS

SEQ ID NO: 356 - protein, 9 aa, Artificial Sequence
  Cris7 and DRA222 VL CDR3 (IMGT)
QQWSRNPPT

SEQ ID NO: 357 - protein, 5 aa, Artificial Sequence
  I2C VH CDR1 (Kabat)
KYAMN

SEQ ID NO: 358 - protein, 19 aa, Artificial Sequence
  I2C VH CDR2 (Kabat)
RIRSKYNNYATYYADSVKD

SEQ ID NO: 359 - protein, 14 aa, Artificial Sequence
  I2C VH CDR3 (Kabat)
HGNFGNSYISYWAY

SEQ ID NO: 360 - protein, 14 aa, Artificial Sequence
  I2C VL CDR1 (Kabat)
GSSTGAVTSGNYPN

SEQ ID NO: 361 - protein, 7 aa, Artificial Sequence
  I2C VL CDR2 (Kabat)
GTKFLAP

SEQ ID NO: 362 - protein, 9 aa, Artificial Sequence
  I2C VL CDR3 (Kabat)
VLWYSNRWV

SEQ ID NO: 363 - protein, 8 aa, Artificial Sequence
  I2C VH CDR1 (IMGT)
GFTFNKYA

SEQ ID NO: 364 - protein, 10 aa, Artificial Sequence
  I2C VH CDR2 (IMGT)
IRSKYNNYAT

SEQ ID NO: 365 - protein, 16 aa, Artificial Sequence
  I2C VH CDR3 (IMGT)
VRHGNFGNSYISYWAY

SEQ ID NO: 366 - protein, 9 aa, Artificial Sequence
  I2C VL CDR1 (IMGT)
TGAVTSGNY

SEQ ID NO: 367 - protein, 3 aa, Artificial Sequence
  I2C VL CDR2 (IMGT)
GTK

SEQ ID NO: 368 - protein, 9 aa, Artificial Sequence
  I2C VL CDR3 (IMGT)
VLWYSNRWV

SEQ ID NO: 369 - protein, 5 aa, Artificial Sequence
  HuM291 VH CDR1 (Kabat)
SYTMH

SEQ ID NO: 370 - protein, 17 aa, Artificial Sequence
  HuM291 VH CDR2 (Kabat)
YINPRSGYTHYNQKLKD

SEQ ID NO: 371 - protein, 11 aa, Artificial Sequence
  HuM291 VH CDR3 (Kabat)
SAYYDYDGFAY

SEQ ID NO: 372 - protein, 10 aa, Artificial Sequence
  HuM291 VL CDR1 (Kabat)
SASSSVSYMN

SEQ ID NO: 373 - protein, 7 aa, Artificial Sequence
  HuM291 VL CDR2 (Kabat)
DTSKLAS

SEQ ID NO: 374 - protein, 9 aa, Artificial Sequence
  HuM291 VL CDR3 (Kabat)
QQWSSNPPT

SEQ ID NO: 375 - protein, 8 aa, Artificial Sequence
  HuM291 VH CDR1 (IMGT)
GYTFISYT

SEQ ID NO: 376 - protein, 8 aa, Artificial Sequence
  HuM291 VH CDR2 (IMGT)
INPRSGYT

SEQ ID NO: 377 - protein, 13 aa, Artificial Sequence
  HuM291 VH CDR3 (IMGT)
ARSAYYDYDGFAY

SEQ ID NO: 378 - protein, 7 aa, Artificial Sequence
  HuM291 VL CDR1 (IMGT)
ASSSVSY

SEQ ID NO: 379 - protein, 3 aa, Artificial Sequence
  HuM291 VL CDR2 (IMGT)
DTS

SEQ ID NO: 380 - protein, 9 aa, Artificial Sequence
  HuM291 VL CDR3 (IMGT)
QQWSSNPPT

SEQ ID NO: 381 - protein, 251 aa, Artificial Sequence
  TSC455 (anti-CD3) TSC394 F87Y scFv
QVQLVQSGPEVKKPGSSVKVSCKASGYTFSRSTMHWVRQAPGQGLEWIGYINPSSAYTNYNQKFKDRVTITADKSTSTAYMELSSLRSEDTAVYYCARPQVHYDYNGFPYWGQGTLVTVSSGGGGSGGGGSGGGGSGGGGSDIQMTQSPSTLSASVGDRVTMTCSASSSVSYMNWYQQKPGKAPKRWIYDSSKLASGVPSRFSGSGSGTEYTLTISSLQPDDFATYYCQQWSRNPPTFGGGTKVEIKRSSS

SEQ ID NO: 382 - protein, 251 aa, Artificial Sequence
  TSC456 (anti-CD3) TSC394 E86D F87Y scFv
QVQLVQSGPEVKKPGSSVKVSCKASGYTFSRSTMHWVRQAPGQGLEWIGYINPSSAYTNYNQKFKDRVTITADKSTSTAYMELSSLRSEDTAVYYCARPQVHYDYNGFPYWGQGTLVTVSSGGGGSGGGGSGGGGSGGGGSDIQMTQSPSTLSASVGDRVTMTCSASSSVSYMNWYQQKPGKAPKRWIYDSSKLASGVPSRFSGSGSGTDYTLTISSLQPDDFATYYCQQWSRNPPTFGGGTKVEIKRSSS

SEQ ID NO: 383 - protein, 121 aa, Artificial Sequence; identical to SEQ ID NO: 25
  TSC455 and TSC456 variable heavy domain

SEQ ID NO: 384 - protein, 108 aa, Artificial Sequence; identical to SEQ ID NO: 26
  TSC455 variable light domain

SEQ ID NO: 385 - protein, 108 aa, Artificial Sequence
  TSC456 variable light domain
DIQMTQSPSTLSASVGDRVTMTCSASSSVSYMNWYQQKPGKAPKRWIYDSSKLASGVPSRFSGSGSGTDYTLTISSLQPDDFATYYCQQWSRNPPTFGGGTKVEIKRS

SEQ ID NO: 386 - protein, 247 aa, Artificial Sequence
  DRA222 (anti-CD3)  scFv
QVQLVESGGGVVQPGRSLRLSCKASGYTFTRSTMHWVRQAPGQGLEWIGYINPSSAYTNYNQKFKDRFTISADKSKSTAFLQMDSLRPEDTGVYFCARPQVHYDYNGFPYWGQGTPVTVSSGGGGSGGGGSGGGGSAQDIQMTQSPSSLSASVGDRVTMTCSASSSVSYMNWYQQKPGKAPKRWIYDSSKLASGVPARFSGSGSGTDYTLTISSLQPEDFATYYCQQWSRNPPTFGGGTKLQITSSS

SEQ ID NO: 387 - protein, 121 aa, Artificial Sequence
  DRA222 variable heavy domain
QVQLVESGGGVVQPGRSLRLSCKASGYTFTRSTMHWVRQAPGQGLEWIGYINPSSAYTNYNQKFKDRFTISADKSKSTAFLQMDSLRPEDTGVYFCARPQVHYDYNGFPYWGQGTPVTVSS

SEQ ID NO: 388 - protein, 107 aa, Artificial Sequence
  DRA222 variable light domain
DIQMTQSPSSLSASVGDRVTMTCSASSSVSYMNWYQQKPGKAPKRWIYDSSKLASGVPARFSGSGSGTDYTLTISSLQPEDFATYYCQQWSRNPPTFGGGTKLQITS

SEQ ID NO: 389 - skipped

SEQ ID NO: 390 - protein, 4 aa, Artificial Sequence
  hinge region
  misc_feature (2)..(3): Xaa can be any naturally occurring amino acid
CXXC

(19) United States
(12) Patent Application Publication
(10) Pub. No.: US 2024/0262920 A1
(43) Pub. Date: Aug. 8, 2024

(54) DOSING REGIMENS FOR PROTEIN THERAPEUTICS

(71) Applicant: Aptevo Research and Development LLC, Seattle, WA (US)

(72) Inventors: Fatih UCKUN, Seattle, WA (US); Scott STROMATT, Seattle, WA (US)

(21) Appl. No.: 18/562,725

(22) PCT Filed: May 20, 2022
(86) PCT No.: PCT/US2022/030323

§ 371 Date: Nov. 20, 2023

Related U.S. Application Data
(60) Provisional application No. 63/253,714, filed on Oct. 8, 2021.
(60) Provisional application No. 63/191,488, filed on May 21, 2021.

(51) Int. Cl. C07K 16/28; A61K 9/00; A61K 31/136; A61K 31/635; A61K 31/704; A61K 31/7068; A61K 39/00; A61P 35/00

(52) U.S. Cl. CPC C07K 16/2866; A61K 9/0019; A61K 31/136; A61K 31/635; A61K 31/704; A61K 31/7068; A61P 35/00; C07K 16/2809; A61K 2039/505; A61K 2039/545; C07K 2317/53; C07K 2317/565; C07K 2317/622

(57) ABSTRACT

Provided herein are methods for treating a cancer, the methods comprising administering to a subject in need thereof: i) a multispecific protein comprising a CD123 binding domain and a CD3 binding domain; and ii) a second anti-cancer agent. The anti-cancer agent used in the methods described herein, may be, for example a chemotherapeutic drug. In some embodiments, the chemotherapeutic drug is venetoclax, azacitidine, decitabine, daunorubicin, cytarabine, idarubicin, mitoxantrone, or etoposide.

### CROSS-REFERENCE TO RELATED APPLICATIONS

This application claims priority to U.S. Provisional Patent Application No. 63/253,714, filed Oct. 8, 2021, and U.S. Provisional Patent Application No. 63/191,488, filed May 21, 2021, each of which is incorporated by reference herein in its entirety for all purposes.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: APVO_069_02WO_SeqList_ST25.txt, date recorded: May 20, 2022, file size: ˜105,091 bytes).

### TECHNICAL FIELD

The disclosure relates to bispecific or multispecific protein therapeutics for treating cancer. More specifically, the disclosure relates bispecific or multispecific proteins comprising a CD123 binding domain and a CD3 binding domain. The disclosure also relates to clinical methods, including dosing regimens, for administration of the protein therapeutics, alone or in combination with one or more additional anti-cancer agents, to a subject in need thereof.

### BACKGROUND

One of the most significant and frequent side effects associated with the use bispecific, T-cell engaging antibodies are neurologic toxicities. Neurologic toxicities may be severe, life-threatening, or fatal. Another potential complication associated with treatment using such antibodies is a systemic inflammatory syndrome known as Cytokine Release Syndrome (CRS). Accordingly, there is a need for dosing strategies to mitigate the risk associated with the effects of cytokine release and other toxicities in patients treated with bispecific and multispecific therapeutics that act by T-cell engagement (i.e., T-cell engagers). This class of therapeutics includes bispecific therapeutics that target CD123 and CD3. mAb14045 (Xencor), a CD123×CD3 bispecific antibody molecule being evaluated in patients with relapsed or refractory acute myeloid leukemia and other CD123-expressing hematologic malignancies, was placed on a partial clinical hold by the FDA in 2019 due to the deaths of two patients in a Phase I trial, including one death caused by CRS.

Dosing strategies designed to reduce the likelihood of severe effects of cytokine release, including CRS, may fail to be therapeutically effective. Accordingly, a need remains for methods to deliver a therapeutically effective dose of a T-cell engager (such as a CD123×CD3 therapeutic) to a patient in a manner that mitigates risk of toxicity, including cytokine toxicity.

### SUMMARY

Described herein are methods for treating cancer, including dosing strategies. The methods may comprise administering to a subject in need thereof a multispecific protein, such as a multispecific protein comprising a CD123 binding domain and a CD3 binding domain. In some embodiments, the multispecific protein is administered in combination with a second anti-cancer agent, such as a chemotherapeutic drug. The dosing strategies described herein may provide therapeutic effects while minimizing the likelihood of neurologic toxicities and/or cytokine release syndrome.

Accordingly, in some embodiments, provided herein is a method for treating a cancer, the method comprising administering to a subject in need thereof: i) a multispecific protein comprising a CD123 binding domain and a CD3 binding domain; and ii) a second anti-cancer agent. In some embodiments, the method further comprises administering to the subject a third anti-cancer agent. The anti-cancer agents used in the methods described herein may be, for example chemotherapeutic drugs. In some embodiments, the chemotherapeutic drug is venetoclax, azacitidine, decitabine, daunorubicin, cytarabine, idarubicin, mitoxantrone, or etoposide.

In some embodiments, provided herein is a method for treating a cancer, the method comprising administering to a subject in need thereof a multispecific protein comprising a CD123 binding domain and a CD3 binding domain.

In some embodiments, the multispecific protein comprises: a dimer of two identical polypeptides, wherein each polypeptide comprises, in order from amino-terminus to carboxyl-terminus, or in order from carboxyl-terminus to amino-terminus: (i) a CD123 binding domain, (ii) a hinge region, (iii) an immunoglobulin constant region, and (iv) a CD3 binding domain. In some embodiments, the polypeptide comprises, from N-terminus to C-terminus, the CD123 binding domain, the hinge region, the immunoglobulin constant region, and the CD3 binding domain. In some embodiments, at least one of the CD123 and the CD3 binding domains comprises: (i) an immunoglobulin heavy chain variable region (VH) comprising HCDR1, HCDR2, and HCDR3; and (ii) an immunoglobulin light chain variable region (VL) comprising LCDR1, LCDR2, and LCDR3. In some embodiments, the CD123 binding domain is a scFv comprising: a HCDR1 that comprises SEQ ID NO: 10, a HCDR2 that comprises SEQ ID NO: 11, and a HDCR3 that comprises SEQ ID NO: 12; and a LCDR1 that comprises SEQ ID NO: 13, a LCDR2 that comprises SEQ ID NO: 14, and a LCDR3 that comprises SEQ ID NO: 15. In some embodiments, the CD123 binding domain is a scFv comprising: a VH comprising a sequence at least 90%, at least 95%, or 100% identical to SEQ ID NO: 136, and a VL comprising a sequence at least 90%, at least 95%, or 100% identical to SEQ ID NO: 134. In some embodiments, the CD123 binding domain is a scFv, and wherein the scFv comprises a sequence at least 90%, at least 95%, or 100% identical to SEQ ID NO: 27. In some embodiments, the CD3 binding domain is a scFv comprising: a HCDR1 that comprises SEQ ID NO: 19, a HCDR2 that comprises SEQ ID NO: 20, and a HDCR3 that comprises SEQ ID NO: 21; and a LCDR1 that comprises SEQ ID NO: 22, a LCDR2 that comprises SEQ ID NO: 23, and a LCDR3 that comprises SEQ ID NO: 24. In some embodiments, the CD3 binding domain is a scFv that comprises: a VH comprising a sequence at least 90%, at least 95%, or 100% identical to SEQ ID NO: 383 or 387, and a VL comprising a sequence at least 90%, at least 95%, or 100% identical to SEQ ID NO: 384. In some embodiments, the CD3 binding domain is a scFv that comprises a sequence at least 90%, at least 95%, or 100% identical to SEQ ID NO: 27. In some embodiments, each polypeptide comprises a sequence at least 90%, at least 95%, or 100% identical to SEQ ID NO: 31.

In some embodiments, the multispecific protein is administered to the subject by IV infusion. In some embodiments, the multispecific protein is administered the subject by IV infusion at a dose of 0.3, 1, 3, 6, 9, 12, 18, 20, 24, 30, 36, 48, 50, 60, 75, or 100 μg. In some embodiments, the multispecific protein is administered once or twice per week. In some embodiments, the second anti-cancer agent is administered to the subject orally or by IV infusion.

In some embodiments, the multispecific protein is administered to the subject by IV infusion during a first 28-day cycle; wherein 6 μg of the multispecific protein is administered on day 8; 12 μg of the multispecific protein is administered on day 15; and 18 μg of the multispecific protein is administered on day 22 of the first 28-day cycle. In some embodiments, the multispecific protein is administered to the subject by IV infusion during at least one additional 28-day cycle following the first 28-day cycle, wherein 18 μg of the multispecific protein is administered on days 1, 8, 15, and 22 of the at least one additional 28-day cycle. In some embodiments, cytarabine is administered intravenously on days 1-5 of the first 28-day cycle, and days 1-5 of at least one additional 28-day cycle. In some embodiments, the dose of cytarabine is about 1 g/m². In some embodiments, mitoxantrone, etoposide, and cytarabine are administered intravenously on days 1-6 of the first 28-day cycle, and at least one additional 28-day cycle. In some embodiments, the dose of mitoxantrone is about 6 mg/m²/day, the dose of etoposide is about 80 mg/m²/day, and the dose of cytarabine is about 1 g/m²/day.

In some embodiments, the multispecific protein is administered to the subject by IV infusion during a first 28-day cycle; wherein 6 μg of the multispecific protein is administered on day 15; and 12 μg of the multispecific protein is administered on day 22 of the first 28-day cycle. In some embodiments, the multispecific protein is administered to the subject by IV infusion during at least one additional 28-day cycle following the first 28-day cycle, wherein 18 μg of the multispecific protein is administered on days 1, 8, 15, and 22 of the at least one additional 28-day cycle. In some embodiments, venetoclax is administered orally on days 1-21 of the first 28-day cycle, and days 1-21 of at least one additional 28-day cycle. In some embodiments, the dose of venetoclax is from about 100 to about 400 mg/day. In some embodiments, azacytidine is administered intravenously on days 1-7 of the first 28-day cycle, and at least one additional 28-day cycle. In some embodiments, the dose of azacytidine is about 75 mg/m².

In some embodiments, the multispecific protein is administered to the subject by IV infusion during a first 28-day cycle, wherein 6 μg of the multispecific protein is administered on day 1; 8 μg of the multispecific protein is administered on day 12; 18 μg of the multispecific protein is administered on day 15; and 18 μg of the multispecific protein is administered on day 22 of the first 28-day cycle. In some embodiments, the multispecific protein is administered to the subject by IV infusion during at least one additional 28-day cycle following the first 28-day cycle, wherein 18 μg of the multispecific protein is administered on days 1, 8, 15, and 22. In some embodiments, cytarabine is administered by intravenous infusion on days 1-7 of the first 28-day cycle, and days 1-7 of at least one additional 28-day cycle. In some embodiments, the dose of cytarabine is from about 100 to about 200 mg/m². In some embodiments, idarubicin is administered by intravenous infusion on days 1-3 of the first 28-day cycle, and days 1-3 of at least one additional 28-day cycle. In some embodiments, the dose of idarubicin is about 12 mg/m².

In some embodiments, the multispecific protein is administered to the subject by IV infusion during a first 28-day cycle, wherein 6 μg of the multispecific protein is administered on day 1; 8 μg of the multispecific protein is administered on day 12; 18 μg of the multispecific protein is administered on day 15; and 18 μg of the multispecific protein is administered on day 22 of the first 28-day cycle. In some embodiments, the multispecific protein is administered to the subject by IV infusion during at least one additional 28-day cycle following the first 28-day cycle, wherein 18 μg of the multispecific protein is administered on days 1, 8, 15, and 22 of the at least one additional 28-day cycle. In some embodiments, azacytidine is administered orally on days 1-14 of the first 28-day cycle, and at least one additional 28-day cycle. In some embodiments, the dose of azacytidine is about 300 mg/day.

In some embodiments, the cancer is a carcinoma or sarcoma. In some embodiments, the cancer is melanoma, kidney cancer, pancreatic cancer, lung cancer, intestinal cancer, prostate cancer, breast cancer, liver cancer, brain cancer, colon cancer, ovarian cancer, or hematological cancer. In some embodiments, wherein the cancer is acute myeloid leukemia (AML), myelodysplastic syndrome (MDS), hairy cell leukemia (HCL), blastic plasmacytoid dendritic cell neoplasm, B-cell acute lymphoblastic leukemia (ALL), or chronic myeloid leukemia (CML).

### BRIEF DESCRIPTION OF THE DRAWINGS

FIG. 1A and FIG. 1B are schematics showing the structures of exemplary therapeutic proteins for use with the compositions and methods of the disclosure. FIG. 1A shows a homodimeric protein comprising two identical polypeptides each comprising a CD3 binding domain and an Fc domain. FIG. 1B shows a homodimeric protein comprising two identical polypeptides each comprising a tumor binding domain (e.g., a CD123 binding domain), an Fc domain, and a CD3 binding domain. An exemplary CD123×CD3 bispecific therapeutic protein is referred to herein as TRI130.

FIG. 2 is a schematic that shows the design of a Phase 1/1b dose escalation clinical study, wherein TRI130 is administered to patients with relapsed or refractory acute myeloid leukemia (AML) or myelodysplastic syndrome (MDS).

FIG. 3A-3D show percentage of blasts in bone marrow aspirates, plotted over time for patients in the Phase 1/1b study described in FIG. 2 and Example 2. Data is graphed for patients in cohorts receiving a highest dose of ≥12 μg. N=14 patients evaluable for changes from baseline. Cohorts 6a and 6b tested different step dosing regimens, as shown in Table 10.

FIG. 4A-4D show serum concentrations of interleukin-6 (IL-6, FIG. 4A), interleukin-10 (IL-10, FIG. 4B), interferon-gamma (IFN-γ, FIG. 4C) and tumor necrosis factor-alpha (TNF-α, FIG. 4D) in patient samples from scheduled blood collections (pre-dose, about 15-30 minutes post-dose, and about 20-26 hours post-dose; N=26) in the Phase 1/1b study described in FIG. 2 and Example 2. The scheduled blood collections were collected at the first administration of the highest planned dose for each patient. Peak cytokine levels observed at unscheduled collections during IRR/CRS events are shown for comparison (N=6 events in 4 patients).

FIG. 5A-5E are schematics showing dosing regimens used in Cohort 1-Cohort 5 of the expansion study described in Example 3. It will be understood by those of skill in the art that the dosing regimens illustrated in FIG. 5A-5E may be used to administer any of the multispecific proteins disclosed herein.

FIG. 6A provides pharmacokinetic (PK) data. TRI130 concentration is shown in patients from Cohort 6a of the dose escalation study at various study time points. The horizontal hashed line represents lower limit of quantification (LLOQ) of assay. All other measurements were within detection limits, but not quantifiable range. The dotted vertical lines represent end of treatment (EOT) for each respective patient.

FIG. 6B shows anti-drug antibody (ADA) response values and titer in patients from Cohort 6a of the dose escalation study at various study time points. The dotted vertical lines represent EOT for each respective patient. The open symbols indicate follow-up samples.

FIG. 7A provides PK data. TRI130 concentration is shown in various patients from Cohort 6b of the dose escalation study at various study time points. The dotted vertical lines represent end of treatment (EOT) for each respective patient.

FIG. 7B shows ADA response values and titer in various patients from Cohort 6a of the dose escalation study at various study time points. The dotted vertical lines represent EOT for each respective patient. The open symbols indicate follow-up samples.

FIG. 8A shows PK simulations, comparing TRI130 concentration over time in Cohort A as compared to Cohort 7. FIG. 8B shows PK simulations, comparing TRI130 concentration overtime in Cohort B as compared to Cohort 7. FIG. 8C shows PK simulations, comparing TRI130 concentration over time in Cohort C as compared to Cohort 7. FIG. 8D shows PK simulations, comparing TRI130 concentration over time in Cohort D as compared to Cohort 7. Traces from these simulations are overlaid in FIG. 8E.

FIG. 9A shows estimated maximum concentration (Cmax), maximum time (Tmax), minimum concentration (Cmin), and area under the curve (AUC) for various cohorts. FIG. 9B shows the ratio of Cmax to Cmin at Cmax, AUC:Cmax, and highest Cmax:Cmin in cycle 1 for various cohorts.

FIG. 10 shows depletion of circulating putative AML-LSC cells in a VENAZA-resistant relapsed AML patient receiving TRI130 monotherapy. The numbers in the left upper corner of each graph in the first and second column of graphs represent the total LSC numbers, of which the vast majority co-expressed both CD123 and CD33. Virtually all of the CD34+CD38−cells were CD123+ and CD33+ consistent with AML. The size of this CD123+CD33+CD34+CD38−AML LSC population indicated with the arrow in Panel A, third column, was significantly reduced by TRI130 monotherapy. See also FIG. 15.

FIG. 11 shows circulating AML-LSC cells in a VENAZA-resistant relapsed AML patient receiving TRI130 monotherapy. Virtually all of the CD34+CD38− cells were CD123+ and CD33+, consistent with AML. The size of the CD123+CD33+CD34+CD38− AML LSC population (indicated with the arrow in the third column of graphs), did not significantly change during the course of TRI130 monotherapy. See also FIG. 15.

FIG. 12 shows the onset and duration of stable disease (SD), PR, CR, clearance of peripheral blasts, and onset of PD, each indicated with specific symbols. Arrow indicates that the patient is alive. See also FIG. 13A-13B.

FIG. 13A-13B is a table showing patient characteristics for AML patients (FIG. 13A) and MDS patients (FIG. 13B) enrolled in the study described in Example 4. C: Cycle number; D: Day number in the specific cycle; AZA: Azacitidine; MDS: MDS-rf: MDS-related features; abn: abnormalities; AZA: azacytidine; MDS: myelodysplastic syndrome; Myelodysplastic Syndromes; PD: Progressive disease; IM-1: Intermediate-1; IM-2: Intermediate-2; SD: stable disease; NA: not applicable; M: male; F: female; C: Caucasian; A: Asian; IPSS: International Prognosis Scoring System; 2016 WHO myelodysplastic syndrome subtypes: MDS with excess blasts (MDS-EB); *UPN09 had multiple transient Grade 3-4 AEs that have resolved; She had tumor lysis syndrome (TLS) Grade 3 on C1 D2, lasting 2 days; TLS Grade 3 on C4D1 lasting 2 days; Anemia, Grade 3 on C5D1 lasting 8 days, Anemia Grade 3 on C7D22 lasting 8 days, Anemia Grade 3 on C6D22 lasting 12 days, Anemia Grade 3 on C7D8 lasting 4 days; decreased platelet count on C4D15 Grade 3 lasting 7 days, decreased platelet count Grade 4 on C4D22 lasting 50 days, decreased platelet count Grade 3 on C6D15 lasting 2 days, decreased platelet count Grade 4 on C6D22 lasting 33 days; hyperglycemia Grade 3 on C5D5 lasting 2 days. $$UPN09: Anemia (Hgb 9.7 g/dL), Thrombocytopenia (Plt 30,000/μL), Intermediate risk karyotype, 47,XX+8, Multiple genetic mutations (NRAS, ASXL-1, PTPNII, RUNX1, STAG2), progressed to CMML-MPN with an absolute monocyte count 17,600/μL around Cycle 10. UPN10: Anemia (Hgb 8.0 g/dL), thrombocytopenia (Plt: 9,000/μL), severe neutropenia (ANC: 0.29×103/μL), 7.7% blasts in bone marrow, cytogenetics not available. Achieved a marrow CR with bone marrow myeloblast count of 2.4%. UPN-11: Anemia (Hgb 9.3 g/dL, thrombocytopenia (Plt: 23,000/μL) (—non-severe neutropenia with ANC 1.3×103/μL), intermediate risk cytogenetics with MDS related aberrations 11q- and -7, 11.3% blasts in the bone marrow. Achieved a marrow CR with bone marrow myeloblast count down to 0% at C2D1. UPN12: No pancytopenia (Hgb 11 g/dL, Plt: 100,000/μL, ANC: 1.1×103/μL), Favorable karyotype: 46, XY, 8.2% blasts in bone marrow. Achieved a marrow CR with bone marrow myeloblast count reduced to 2% at C2D1. UPN13: Anemia (Hgb 7.2 g/dL), thrombocytopenia (Plt: 8,000/μL), severe neutropenia (ANC: 0.5×103/μL), 5% blasts in bone marrow, complex karyotype (poor risk category): 46,XY,der(12;19)(q10;p10), +mar[8]/46,XY, del(20)(q11.2q13.1). Intermediate risk karyotype with 12(p) aberration on cytogenetics. 5% blasts in bone marrow. UPN14: Anemia (Hgb 9.3/dL), Neutropenia (ANC: 0×103/μL), Intermediate risk karyotype with t(1;2)(p36.3;p21) on cytogenetics, 6% blasts in bone marrow.

FIG. 14 is a table showing that TRI130 activates TH1 and TH2 cells from relapsed/refractory AML and MDS patients. E: early sample obtained at beginning of cycle 2; L: late sample obtained during cycle 2. *IL-10 induction was observed in UPN01 (46-fold), UPN05 (8.3-fold), UPN06 (17.3-fold), and UPN10 (24.5-fold). Only two MDS patients with SD as their BOR had no significant IL-10 induction. **IFN-γ induction was observed in UPN01 and UPN05. ***IL-5 induction was observed on UPN06 only.

FIG. 15 is a table showing pharmacodynamic effects of TRI130 on circulating putative LSC.

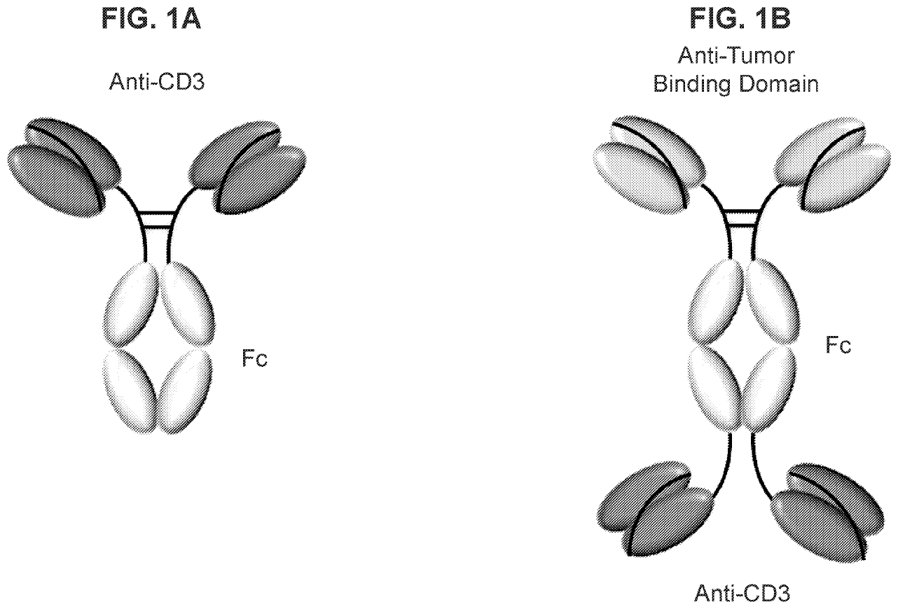

### DETAILED DESCRIPTION

One of the most significant and frequent side effects associated with the use bispecific, T-cell engaging antibodies are neurologic toxicities. Neurologic toxicities may be severe, life-threatening, or fatal. The instant application describes multispecific proteins comprising a CD123 binding domain and a CD3 binding, including TRI130. Clinical data is provided which shows that TRI130 is not associated with serious neurologic toxicity. Another potential complication associated with treatment using bispecific, T-cell engaging antibodies is a systemic inflammatory syndrome known as Cytokine Release Syndrome (CRS). TRI130 produced CRS in some patients, however it was managed using standard CRS treatments. TRI130 showed signs of clinical activity, sustained stabilization of leukemia, a response that consequently deepened to partial remission and complete remission in two difficult to treat relapsed/refractory AML patients.

CD123, although highly expressed on AML blasts, is also expressed on normal bone marrow hematopoietic stem cells and myeloid progenitor cells that give rise to the infection-fighting white blood cells. Therefore, treatment platforms targeting CD123 have been frequently associated with a prolonged and profound decrease white blood cell counts, known as neutropenia, and infections, especially pneumonias. This common side effect which overlaps with the blood count lowering side effects of standard chemotherapy drugs is among the main hurdles impeding the desired incorporation of CD123 targeting drugs into contemporary frontline as well as second-line standard of care treatment regimens. Thus, there is an urgent need for active new drugs for treating AML patients that are capable of destroying leukemia cells without damaging the normal bone marrow cells. Notably, the instant inventors unexpectedly discovered that—unlike other CD123 targeting drugs—TRI130 does not cause severe or prolonged neutropenia at doses that resulted in complete remissions, even after months of weekly infusions. This unique characteristic allows for the use of TRI130 in combination with one or more additional anti-cancer agents, such as chemotherapeutic drugs.

These and other aspects of the invention will be described in further detail below.

The section headings used herein are for organizational purposes only and are not to be construed as limiting the subject matter described. All documents, or portions of documents, cited herein, including but not limited to patents, patent applications, articles, books, and treatises, are hereby expressly incorporated by reference in their entirety for any purpose. In the event that one or more of the incorporated documents or portions of documents define a term that contradicts that term's definition in the application, the definition that appears in this application controls. However, mention of any reference, article, publication, patent, patent publication, and patent application cited herein is not, and should not be taken as an acknowledgment, or any form of suggestion, that they constitute valid prior art or form part of the common general knowledge in any country in the world.

In the present description, any concentration range, percentage range, ratio range, or integer range is to be understood to include the value of any integer within the recited range and, when appropriate, fractions thereof (such as one tenth and one hundredth of an integer), unless otherwise indicated. It should be understood that the terms “a” and “an” as used herein refer to “one or more” of the enumerated components unless otherwise indicated. The use of the alternative (e.g., “or”) should be understood to mean either one, both, or any combination thereof of the alternatives. As used herein, the terms “include” and “comprise” are used synonymously. In addition, it should be understood that the polypeptides comprising the various combinations of the components (e.g., domains or regions) and substituents described herein, are disclosed by the present application to the same extent as if each polypeptide was set forth individually. Thus, selection of particular components of individual polypeptides is within the scope of the present disclosure.

### Definitions

The term “about” when immediately preceding a numerical value means±up to 10% of the numerical value. For example, “about 40” means±up to 10% of 40 (i.e., from 36 to 44), for example ±up to 10%, up to 9%, ±up to 8%, ±up to 7%, ±up to 6%, ±up to 5%, ±up to 4%, ±up to 3%, up to 2%, ±up to 1%, up to less than 1%, or any other value or range of values therein.

As used herein, substantially has its ordinary meaning as used in the art. For example, “substantially” may mean “significantly,” “considerably,” “largely,” “mostly,” or “essentially.” In some embodiments, “substantially” may refer to at least about 50%, at least about 60%, at least about 70%, at least about 80%, at least about 90%, at least about 95%, at least about 96%, at least about 97%, at least about 98%, or at least about 99%.

The term “CD123” may refer to any isoform of CD123, also known as Cluster of Differentiation 123, Interleukin-3 receptor alpha chain, and IL3RA. CD123 associates with the beta chain of the interleukin-3 receptor to form the receptor. CD123 is a type I transmembrane glycoprotein, with an extracellular domain comprising a predicted Ig-like domain and two FnIII domains. The CD123-binding domains of the disclosure bind to the extracellular domain of CD123. CD123 is also known as the alpha chain of the human interleukin-3 (IL-3) receptor. CD123 is a type I transmembrane glycoprotein and is a member of the cytokine receptor superfamily. The interleukin-3 receptor is a heterodimer formed by CD123 and the beta chain (CD131). IL-3 binds to CD123, and signal transduction is provided by CD131. IL-3 regulates the function and production of hematopoietic and immune cells and stimulates endothelial cell proliferation (Testa et al., Biomark Res. 2:4 (2014)).

CD123 is overexpressed in many hematologic malignancies, including a subset of acute myeloid leukemia (AML), B-lymphoid leukemia, blastic plasmocytoid dendritic neoplasms (BPDCN) and hairy cell leukemia. While most AML patients respond well to initial therapies, the majority of AML patients are ultimately diagnosed with relapsed or refractory disease (Ramos et al., J. Clin. Med. 4:665-695 (2015)). There is a need for molecules targeting CD123 with increased efficiency and potency and reduced adverse effects and that may be used to treat disorders associated with dysregulation of CD123.

“CD3” is known in the art as a multi-protein complex of six chains (see, e.g., Abbas and Lichtman, 2003; Janeway et al., p. 172 and 178, 1999), which are subunits of the T-cell receptor complex. In mammals, the CD3 subunits of the T-cell receptor complex are a CD3γ chain, a CD3δ chain, two CD3ε chains, and a homodimer of CD3ζ chains. The CD3γ, CD3δ, and CD3ε chains are highly related cell surface proteins of the immunoglobulin superfamily containing a single immunoglobulin domain. The transmembrane regions of the CD3γ, CD3δ, and CD3ε chains are negatively charged, which is a characteristic that allows these chains to associate with the positively charged T-cell receptor chains. The intracellular tails of the CD3γ, CD3δ, and CD3ε chains each contain a single conserved motif known as an immunoreceptor tyrosine-based activation motif or ITAM, whereas each CD3ζ chain has three. It is believed the ITAMs are important for the signaling capacity of a TCR complex. CD3 as used in the present disclosure can be from various animal species, including human, monkey, mouse, rat, or other mammals.

“Cytokine release” or “cytokine storm” or “infusion reaction” refers to the release of cytokines from T-cells. When cytokines are released into the circulation, systemic symptoms such as fever, nausea, chills, hypotension, tachycardia, asthenia, headache, rash, scratchy throat, and dyspnea can result. Some patients may experience severe, life-threatening reactions that result from massive release of cytokines. “Reduced” cytokine release refers to the to the reduction in the release of at least one cytokine (e.g., IFN-γ, TNF-α, IL-6, IL-2, IL-8, IL-10, IL-17, GM-CSF, IL-4, IL-12, IL-13 or IL-1β) following administration of a bispecific molecule as disclosed herein, as compared to the OKT3 antibody (which binds CD3) or other CD3 binding bispecific molecule. Reduced cytokine release can be measured using in vitro assays or in vivo assays.

As used herein, the term “step dosing” or “stepped dosing” or similar terms refers to a dosing regimen wherein a multispecific polypeptide as described herein is administered to a patient on at least a first day and a second day, wherein the dose administered to the patient is either kept constant or increased between the first day and the second day. For example, in some step dosing regimens, a patient may be administered a first, a second, a third, and a fourth dose, wherein each dose is administered on different day, and wherein the second dose is greater than the first dose. The third dose may be greater than the second dose, or may be the same as the second dose. The fourth dose may be greater than the third dose, or may be the same as the third dose. In some embodiments, if the patient has an adverse response to a particular dose, the subsequent dose may be reduced.

As used herein, the term “binding domain” or “binding region” refers to the domain, region, portion, or site of a protein, polypeptide, oligopeptide, peptide, antibody, or binding domain derived from an antibody, receptor or ligand that possesses the ability to specifically recognize and bind to a target molecule, such as an antigen, ligand, receptor, substrate, or inhibitor. Exemplary binding domains include, antibodies and antibody-like proteins or domains, antibody heavy and light chain variable regions, and single-chain antibody variable regions (e.g., domain antibodies, sFv, scFv, scFab), receptor ectodomains and ligands (e.g., cytokines, chemokines). In certain embodiments, the binding domain comprises or consists of an antigen binding site (e.g., comprising a variable heavy chain sequence and variable light chain sequence or three light chain complementary determining regions (CDRs) and three heavy chain CDRs from an antibody placed into alternative framework regions (FRs) (e.g., human FRs optionally comprising one or more amino acid substitutions). A variety of assays are known for identifying binding domains of the present disclosure that specifically bind a particular target, including Western blot, ELISA, phage display library screening, and BIACORE® interaction analysis.

A binding domain or protein comprising a binding domain “specifically binds” a target if it binds the target with an affinity or Kₐ (i.e., an equilibrium association constant of a particular binding interaction with units of 1/M) equal to or greater than 10⁵ M⁻¹, while not significantly binding other components present in a test sample. Binding domains can be classified as “high affinity” binding domains and “low affinity” binding domains. “High affinity” binding domains refer to those binding domains with a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. “Low affinity” binding domains refer to those binding domains with a Kₐ of up to 10⁷ M⁻¹, up to 10⁶ M⁻¹, up to 10⁵ M⁻¹. Alternatively, affinity can be defined as an equilibrium dissociation constant (K_{d}) of a particular binding interaction with units of M (e.g., 10⁻⁵ M to 10⁻¹³, or about 500 nM, about 300 nM, about 250 nM, about 200 nM, about 150 nM, about 100 nM, about 50 nM, about 25 nM, about 10 nM, or about 5 nM). Affinities of binding domain polypeptides and single chain polypeptides according to the present disclosure can be readily determined using conventional techniques (see, e.g., Scatchard et al. (1949) Ann. N.Y. Acad. Sci. 51:660; and U.S. Pat. Nos. 5,283,173, 5,468,614, or the equivalent).

As used herein, a “conservative substitution” is recognized in the art as a substitution of one amino acid for another amino acid that has similar properties. Exemplary conservative substitutions are well-known in the art (see, e.g., PCT Application Publication No. WO 97/09433, page 10, published Mar. 13, 1997; Lehninger, Biochemistry, Second Edition; Worth Publishers, Inc. NY:NY (1975), pp. 71-77; Lewin, Genes IV, Oxford University Press, NY and Cell Press, Cambridge, MA (1990), p. 8). In certain embodiments, a conservative substitution includes a leucine to serine substitution.

As used herein, the term “derivative” refers to a modification of one or more amino acid residues of a peptide by chemical or biological means, either with or without an enzyme, e.g., by glycosylation, alkylation, acylation, ester formation, or amide formation.

As used herein, a polypeptide or amino acid sequence “derived from” a designated polypeptide or protein refers to the origin of the polypeptide. In certain embodiments, the polypeptide or amino acid sequence which is derived from a particular sequence (sometimes referred to as the “starting” or “parent” or “parental” sequence) and has an amino acid sequence that is essentially identical to the parent sequence or a portion thereof, wherein the portion consists of at least 10-20 amino acids, at least 20-30 amino acids, or at least 30-50 amino acids, or at least 50-150 amino acids, or which is otherwise identifiable to one of ordinary skill in the art as having its origin in the parent sequence. For example, a binding domain can be derived from an antibody, e.g., a Fab, F(ab′)2, Fab′, scFv, single domain antibody (sdAb), etc.

Polypeptides derived from another polypeptide can have one or more mutations or alterations relative to the parent polypeptide, e.g., one or more amino acid residues which have been substituted with another amino acid residue or which has one or more amino acid insertions or deletions. In such embodiments, polypeptides derived from a parent polypeptide and comprising one or more mutations or alteration are referred to as “variants.” As used herein, the term “variant” or “variants” refers to a polynucleotide or polypeptide with a sequence differing from that of a reference polynucleotide or polypeptide but retaining essential properties thereof. Generally, variant polynucleotide or polypeptide sequences are overall closely similar, and, in many regions, identical to the reference polynucleotide or polypeptide. For instance, a variant polynucleotide or polypeptide may exhibit at least about 70%, at least about 80%, at least about 90%, at least about 91%, at least about 92%, at least about 93%, at least about 94%, at least about 95%, at least about 96%, at least about 97%, at least about 98% or at least about 99% sequence identity compared to the active portion or full-length reference polynucleotide or polypeptide. The polypeptide can comprise an amino acid sequence which is not naturally occurring. Such variations necessarily have less than 100% sequence identity or similarity with the parent polypeptide. In one embodiment, the variant will have an amino acid sequence from about 60% to less than 100% amino acid sequence identity or similarity with the amino acid sequence of the parent polypeptide. In another embodiment, the variant will have an amino acid sequence from about 75% to less than 100%, from about 80% to less than 100%, from about 85% to less than 100%, from about 90% to less than 100%, from about 95% to less than 100% amino acid sequence identity or similarity with the amino acid sequence of the parent polypeptide.

As used herein, the term “sequence identity” refers to a relationship between two or more polynucleotide sequences or between two or more polypeptide sequences. When a position in one sequence is occupied by the same nucleic acid base or amino acid residue in the corresponding position of the comparator sequence, the sequences are said to be “identical” at that position. The percentage sequence identity is calculated by determining the number of positions at which the identical nucleic acid base or amino acid residue occurs in both sequences to yield the number of identical positions. The number of identical positions is then divided by the total number of positions in the comparison window and multiplied by 100 to yield the percentage of sequence identity. Percentage of sequence identity is determined by comparing two optimally aligned sequences over a comparison window. The comparison window for polynucleotide sequences can be, for instance, at least about 20, about 30, about 40, about 50, about 60, about 70, about 80, about 90, about 100, about 110, about 120, about 130, about 140, about 150, about 160, about 170, about 180, about 190, about 200, about 300, about 400, about 500, about 600, about 700, about 800, about 900 or about 1000 or more nucleic acids in length. The comparison window for polypeptide sequences can be, for instance, at least about 20, about 30, about 40, about 50, about 60, about 70, about 80, about 90, about 100, about 110, about 120, about 130, about 140, about 150, about 160, about 170, about 180, about 190, about 200, about 300 or more amino acids in length. In order to optimally align sequences for comparison, the portion of a polynucleotide or polypeptide sequence in the comparison window can comprise additions or deletions termed gaps while the reference sequence is kept constant. An optimal alignment is that alignment which, even with gaps, produces the greatest possible number of “identical” positions between the reference and comparator sequences. Percentage “sequence identity” between two sequences can be determined using the version of the program “BLAST 2 Sequences” which was available from the National Center for Biotechnology Information as of Sep. 1, 2004, which program incorporates the programs BLASTN (for nucleotide sequence comparison) and BLASTP (for polypeptide sequence comparison), which programs are based on the algorithm of Karlin and Altschul (Proc. Natl. Acad. Sci. USA 90(12):5873-5877, 1993). When utilizing “BLAST 2 Sequences,” parameters that were default parameters as of Sep. 1, 2004, can be used for word size (3), open gap penalty (11), extension gap penalty (1), gap dropoff (50), expect value (10) and any other required parameter including but not limited to matrix option. Two nucleotide or amino acid sequences are considered to have “substantially similar sequence identity” or “substantial sequence identity” if the two sequences have at least about 80%, at least about 85%, at least about 90%, at least about 95%, at least about 96%, at least about 97%, at least about 98%, or at least about 99% sequence identity relative to each other.

As used herein, unless otherwise provided, a position of an amino acid residue in a variable region of an immunoglobulin molecule is numbered according to the IMGT numbering convention (Brochet, X, et al, Nucl. Acids Res. (2008) 36, W503-508), and a position of an amino acid residue in a constant region of an immunoglobulin molecule is numbered according to EU nomenclature (Ward et al., 1995 Therap. Immunol. 2:77-94). Other numbering conventions are known in the art (e.g., the Kabat numbering convention (Kabat, Sequences of Proteins of Immunological Interest, 5^{th} ed. Bethesda, MD: Public Health Service, National Institutes of Health (1991)).

As used herein, the term “dimer” refers to a biological entity that consists of two subunits associated with each other via one or more forms of intramolecular forces, including covalent bonds (e.g., disulfide bonds) and other interactions (e.g., electrostatic interactions, salt bridges, hydrogen bonding, and hydrophobic interactions), and is stable under appropriate conditions (e.g., under physiological conditions, in an aqueous solution suitable for expressing, purifying, and/or storing recombinant proteins, or under conditions for non-denaturing and/or non-reducing electrophoresis). A “heterodimer” or “heterodimeric protein,” as used herein, refers to a dimer formed from two different polypeptides. A heterodimer does not include an antibody formed from four polypeptides (i.e., two light chains and two heavy chains). A “homodimer” or “homodimeric protein,” as used herein, refers to a dimer formed from two identical polypeptides. All disclosure of the polypeptide, including characteristics and activities (such as binding and RTCC) should be understood to include the polypeptide in its dimer form as well as other multimeric forms.

When a polypeptide of the disclosure is in dimeric form (i.e., a dimeric protein), it contains two binding sites at the amino-terminus and two binding sites at the carboxyl terminus. The binding domains are thus considered bivalent (i.e., two binding portions at each terminus) when the single chain polypeptides are dimerized.

An “immunoglobulin constant region” or “constant region” is a term defined herein to refer to a peptide or polypeptide sequence that corresponds to or is derived from part or all of one or more constant domains of an immunoglobulin. In certain embodiments, the constant region comprises IgG CH2 and CH3 domains, e.g., IgG1 CH2 and CH3 domains. In certain embodiments, the constant region does not comprise a CH1 domain. In certain embodiments, the constant domains making up the constant region are human. In some embodiments, the constant region of a fusion protein of this disclosure lacks or has minimal effector functions while retaining the ability to bind some Fc receptors such as the neonatal Fc receptor (FcRn) and retaining a relatively long half-life in vivo. For example, the constant region of a fusion protein of this disclosure do not result in, or substantially reduce the induction of antibody-dependent cell-mediated cytotoxicity (ADCC), antibody-dependent cell-mediated phagocytosis (ADCP), complement activation, and/or complement-dependent cytotoxicity (CDC). In other variations, a fusion protein of this disclosure comprises constant domains that retain one or more effector functions, such as of one or both of ADCC and CDC. In certain embodiments, a binding domain of this disclosure is fused to a human IgG1 constant region, wherein the IgG1 constant region has one or more of the following amino acids mutated: leucine at position 234 (L234), leucine at position 235 (L235), glycine at position 237 (G237), glutamate at position 318 (E318), lysine at position 320 (K320), lysine at position 322 (K322), or any combination thereof (numbering according to EU). For example, any one or more of these amino acids can be changed to alanine. In a further embodiment, an IgG1 Fc domain has each of L234, L235, G237, E318, K320, and K322 (according to EU numbering) mutated to an alanine (i.e., L234A, L235A, G237A, E318A, K320A, and K322A, respectively), and optionally an N297A mutation as well (i.e., essentially eliminating glycosylation of the CH2 domain).

The terms “light chain variable region” (also referred to as “light chain variable domain” or “V_{L}”) and “heavy chain variable region” (also referred to as “heavy chain variable domain” or “V_{H}”) refer to the variable binding region from an antibody light and heavy chain, respectively. The variable binding regions are made up of discrete, well-defined sub-regions known as “complementarity determining regions” (CDRs) and “framework regions” (FRs). In one embodiment, the FRs are humanized. The term “CL” refers to an “immunoglobulin light chain constant region” or a “light chain constant region,” i.e., a constant region from an antibody light chain. The term “CH” refers to an “immunoglobulin heavy chain constant region” or a “heavy chain constant region,” which is further divisible, depending on the antibody isotype into CH1, CH2, and CH3 (IgA, IgD, IgG), or CH1, CH2, CH3, and CH4 domains (IgE, IgM). A “Fab” (fragment antigen binding) is the part of an antibody that binds to antigens and includes the variable region and CH1 domain of the heavy chain linked to the light chain via an inter-chain disulfide bond.

As used herein, the term “linker” generally refers to a short polypeptide sequence connecting two sub-domains of a polypeptide. Non-limiting examples of linkers include flexible linkers comprising glycine-serine repeats, and linkers derived from (a) an interdomain region of a transmembrane protein (e.g., a type I transmembrane protein); or (b) an immunoglobulin hinge. In some embodiments, a linker provides a spacer function compatible with interaction of the two sub-binding domains so that the resulting polypeptide retains a specific binding affinity to the same target molecule as an antibody that comprises the same light and heavy chain variable regions. In certain embodiments, a linker is comprised of five to about 35 amino acids, for instance, about 15 to about 25 amino acids. As used herein, the phrase a “linker between CH3 and CH1 or CL” refers to one or more amino acid residues (e.g., about 2-12, about 2-10, about 4-10, about 5-10, about 6-10, about 7-10, about 8-10, about 9-10, about 8-12, about 9-12, or about 10-12) between the C-terminus of a CH3 domain (e.g., a wild type CH3 or a mutated CH3) and the N-terminus of a CH1 domain or CL domain (e.g., Cκ).

In some embodiments, depending on context, a linker may refer to (1) a polypeptide region between V_{H} and V_{L} regions in a single-chain Fv (scFv) or (2) a polypeptide region between a first binding domain and a second binding domain in a multispecific polypeptide comprising two binding domains. In the later example, wherein a linker connects two or more binding domains, such a linker is referred to herein as a “Fc-binding domain linker.” In some embodiments, a Fc-binding domain linker may directly link or connect two or more binding domains, resulting in a construct comprising the following structure: binding domain—Fc-binding domain linker—binding domain. In some embodiments, the multispecific polypeptides described herein comprise, in order from amino-terminus to carboxyl-terminus (i) a first binding domain, (ii) a Fc-binding domain linker, and (iii) a second binding domain. In some embodiments, a multispecific polypeptide comprises, in order from amino-terminus to carboxyl-terminus (i) a second binding domain, (ii) a Fc-binding domain linker, and (iii) a first binding domain. In some embodiments, a Fc-binding domain linker may link or connect two or more binding domains by linking at least one binding domain to a non-binding domain polypeptide, such as an immunoglobulin Fc domain (i.e., a polypeptide comprising the structure: Ig hinge—Ig constant region). In such embodiments, the resulting constructs may comprise the following structure: binding domain—Fc domain—Fc-binding domain linker—binding domain. In some embodiments, the multispecific polypeptides described herein comprise, in order from amino-terminus to carboxyl-terminus: (i) a first binding domain, (ii) a hinge region, (iii) an immunoglobulin constant region, (iv) a Fc-binding domain linker, and (v) a second binding domain. In some embodiments, a multispecific polypeptide comprises, in order from amino-terminus to carboxyl-terminus (i) a second binding domain, (ii) a Fc-binding domain linker, (iii) an immunoglobulin constant region, (iv) a hinge region, and (v) a first binding domain. A polypeptide region between an immunoglobulin constant region and a second binding domain in a multispecific polypeptide comprising two binding domains (e.g., a Fc-binding domain linker) may also be referred to as a “carboxyl-terminus linker” or an “amino-terminus linker” depending on the orientation of the domains within the multispecific polypeptide. Non-limiting examples of linkers are provided in Table 1.

In some embodiments, a “hinge” or a “hinge region” refers to a polypeptide derived from an immunoglobulin hinge region and located between a binding domain and an immunoglobulin constant region in a polypeptide described herein. A “wild-type immunoglobulin hinge region” refers to a naturally occurring upper and middle hinge amino acid sequences interposed between and connecting the CH1 and CH2 domains (for IgG, IgA, and IgD) or interposed between and connecting the CH1 and CH3 domains (for IgE and IgM) found in the heavy chain of an antibody. In certain embodiments, a wild type immunoglobulin hinge region sequence is human, and can comprise a human IgG hinge region (e.g., and IgG1, IgG2, IgG3, or IgG4 hinge region).

An “altered immunoglobulin hinge region” or “variant immunoglobulin hinge region” refers to a hinge region polypeptide with one or more mutations, substitutions, insertions, or deletions compared to a corresponding parental wild-type immunoglobulin hinge region. In certain embodiments, an altered immunoglobulin hinge region is at least about 70% identical to a wild-type immunoglobulin hinge region (e.g., at least about 70%, at least about 75%, at least about 80%, at least about 85%, at least about 90%, at least about 95%, at least about 97%, at least about 98%, or at least about 99% identical). In certain embodiments, an altered immunoglobulin hinge region is a fragment of a wild type immunoglobulin hinge region that has a length of about 5 amino acids (e.g., about 5, about 6, about 7, about 8, about 9, about 10, about 11, about 12, about 13, about 14, about 15, about 16, about 17, about 18, about 19, about 20, or more amino acids) up to about 120 amino acids (for instance, having a length of about 10 to about 40 amino acids or about 15 to about 30 amino acids or about 15 to about 20 amino acids or about 20 to about 25 amino acids). Typically, an altered immunoglobulin hinge region that is a fragment of a wild type immunoglobulin hinge region comprises an IgG core hinge region (e.g., a polypeptide comprising the sequence C-X-X-C, wherein X is any amino acid (SEQ ID NO: 390)) as disclosed in U.S. Patent Application Publication Nos. 2013/0129723 and 2013/0095097. Non-limiting examples of hinges are provided in Table 2.

As used herein, the term “humanized” refers to a process of making an antibody or immunoglobulin binding proteins and polypeptides derived from a non-human species (e.g., mouse or rat) less immunogenic to humans, while still retaining antigen-binding properties of the original antibody, using genetic engineering techniques. In some embodiments, the binding domain(s) of an antibody or immunoglobulin binding proteins and polypeptides (e.g., light and heavy chain variable regions, Fab, scFv) are humanized. Non-human binding domains can be humanized using techniques known as CDR grafting (Jones et al., Nature 321:522 (1986)) and variants thereof, including “reshaping” (Verhoeyen, et al., 1988 Science 239:1534-1536; Riechmann, et al., 1988 Nature 332:323-337; Tempest, et al., Bio/Technol 1991 9:266-271), “hyperchimerization” (Queen, et al., 1989 Proc Natl Acad Sci USA 86:10029-10033; Co, et al., 1991 Proc Natl Acad Sci USA 88:2869-2873; Co, et al., 1992 J Immunol 148:1149-1154), and “veneering” (Mark, et al., “Derivation of therapeutically active humanized and veneered anti-CD18 antibodies.” In: Metcalf B W, Dalton B J, eds. Cellular adhesion: molecular definition to therapeutic potential. New York: Plenum Press, 1994: 291-312). If derived from a non-human source, other regions of the antibody or immunoglobulin binding proteins and polypeptides, such as the hinge region and constant region domains, can also be humanized.

An “immunoglobulin dimerization domain” or “immunoglobulin heterodimerization domain”, as used herein, refers to an immunoglobulin domain of a polypeptide chain that preferentially interacts or associates with a different immunoglobulin domain of a second polypeptide chain, wherein the interaction of the different immunoglobulin heterodimerization domains substantially contributes to or efficiently promotes heterodimerization of the first and second polypeptide chains (i.e., the formation of a dimer between two different polypeptide chains, which is also referred to as a “heterodimer”). The interactions between immunoglobulin heterodimerization domains “substantially contributes to or efficiently promotes” the heterodimerization of first and second polypeptide chains if there is a statistically significant reduction in the dimerization between the first and second polypeptide chains in the absence of the immunoglobulin heterodimerization domain of the first polypeptide chain and/or the immunoglobulin heterodimerization domain of the second polypeptide chain. In certain embodiments, when the first and second polypeptide chains are co-expressed, at least 60%, at least about 60% to about 70%, at least about 70% to about 80%, at least 80% to about 90%, about 91%, about 92%, about 93%, about 94%, about 95%, about 96%, about 97%, about 98%, or about 99% of the first and second polypeptide chains form heterodimers with each other. Representative immunoglobulin heterodimerization domains include an immunoglobulin CH1 domain, an immunoglobulin CL domain (e.g., Cκ or Cλ isotypes), or derivatives thereof, including wild type immunoglobulin CH1 and CL domains and altered (or mutated) immunoglobulin CH1 and CL domains, as provided therein.

The terms patient and subject are used interchangeably herein. As used herein, the term “patient in need” or “subject in need” refers to a patient or subject at risk of, or suffering from, a disease, disorder or condition that is amenable to treatment or amelioration with a binding protein or multispecific polypeptide or a composition thereof provided herein. “Patient” and “subject” are used interchangeably herein.

As used herein, the term “pharmaceutically acceptable” refers to molecular entities and compositions that do not generally produce allergic or other serious adverse reactions when administered using routes well known in the art. Molecular entities and compositions approved by a regulatory agency of the Federal or a state government or listed in the U.S. Pharmacopeia or other generally recognized pharmacopeia for use in animals, and more particularly in humans are considered to be “pharmaceutically acceptable.”

As used herein, the terms “nucleic acid,” “nucleic acid molecule,” or “polynucleotide” refer to deoxyribonucleotides or ribonucleotides and polymers thereof in either single- or double-stranded form. Unless specifically limited, the terms encompass nucleic acids containing analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions can be achieved by generating sequences in which the third position of one or more selected (or all) codons is substituted with mixed-base and/or deoxyinosine residues (Batzer et al. (1991) Nucleic Acid Res. 19:5081; Ohtsuka et al. (1985) J. Biol. Chem. 260:2605-2608; Cassol et al. (1992); Rossolini et al. (1994) Mol. Cell. Probes 8:91-98). The term nucleic acid is used interchangeably with gene, cDNA, and mRNA encoded by a gene. As used herein, the terms “nucleic acid,” “nucleic acid molecule,” or “polynucleotide” are intended to include DNA molecules (e.g., cDNA or genomic DNA), RNA molecules (e.g., mRNA), analogs of the DNA or RNA generated using nucleotide analogs, and derivatives, fragments and homologs thereof.

The term “expression” refers to the biosynthesis of a product encoded by a nucleic acid. For example, in the case of nucleic acid segment encoding a polypeptide of interest, expression involves transcription of the nucleic acid segment into mRNA and the translation of mRNA into one or more polypeptides.

The terms “expression unit” and “expression cassette” are used interchangeably herein and denote a nucleic acid segment encoding a polypeptide of interest and capable of providing expression of the nucleic acid segment in a host cell. An expression unit typically comprises a transcription promoter, an open reading frame encoding the polypeptide of interest, and a transcription terminator, all in operable configuration. In addition to a transcriptional promoter and terminator, an expression unit can further include other nucleic acid segments such as, e.g., an enhancer or a polyadenylation signal.

The term “expression vector,” as used herein, refers to a nucleic acid molecule, linear or circular, comprising one or more expression units. In addition to one or more expression units, an expression vector can also include additional nucleic acid segments such as, for example, one or more origins of replication or one or more selectable markers. Expression vectors are generally derived from plasmid or viral DNA, or can contain elements of both.

As used herein, a “polypeptide,” “polypeptide chain,” or “protein” refers to a contiguous arrangement of covalently linked amino acids. Polypeptides can form one or more intrachain disulfide bonds. With regard to polypeptides as described herein, reference to modifications or alterations of amino acid residues corresponding to those specified by SEQ ID NO includes post-translational modifications of such residues. The terms polypeptide and protein also encompass embodiments where two polypeptide chains link together in a non-linear fashion, such as via an interchain disulfide bond. For example, a native immunoglobulin molecule is comprised of two heavy chain polypeptides and two light chain polypeptides.

As used herein, a “multispecific polypeptide” refers to a polypeptide comprising two or more binding domains each capable of specifically binding to a target antigen. For example, the polypeptides described herein may comprise 2, 3, 4, or more binding domains and may be able to bind 2, 3, 4, or more target antigens. In some embodiments, a multispecific polypeptide is a bispecific polypeptide. Herein, a “bispecific polypeptide” comprises two binding domains and capable of binding to two distinct target antigens. In some embodiments, the bispecific polypeptides described herein comprise a first binding domain that specifically binds to a cell surface antigen expressed on a target cell. In some embodiments, the bispecific polypeptides described herein comprise a binding domain that specifically binds to a cell surface antigen expressed on an effector cell. A binding domain may be derived from an antibody (e.g., a variable heavy chain and/or variable light change, scFv), a ligand, or a receptor.

Multispecific polypeptides are disclosed, for instance, in PCT Publication Nos. WO 2007/146968; WO 2010/040105; WO 2010/003108; WO 2016/094873; WO 2017/053469; U.S. Patent Application Publication No. 2006/0051844; and U.S. Pat. Nos. 7,166,707; and 8,409,577, which are each incorporated herein by reference in their entirety. In certain embodiments, the multispecific polypeptides described herein are bispecific polypeptides and may comprise an scFv-Fc-scFv structure, also referred to herein as an ADAPTIR™ polypeptide. The structure of a polypeptide comprising such a structure comprises, from N-terminus to C-terminus: a first scFv binding domain—an immunoglobulin (Ig) hinge region—an Ig constant region—a second scFv binding domain.

A protein or polypeptide may be an antibody or an antigen-binding fragment of an antibody. In some embodiments, a protein may be a recombinant multispecific protein. In other embodiments, a multispecific protein may be produced by chemically linking two different monoclonal antibodies or by fusing two hybridoma cell lines to produce a hybrid-hybridoma. Other multivalent formats that can be used include, for example, quadromas, Kλ-bodies, dAbs, diabodies, TandAbs, nanobodies, Small Modular ImmunoPharmaceutials (SMIPS™), DOCK-AND-LOCKs® (DNLs®), CrossMab Fabs, CrossMab V_{H}-VLs, strand-exchange engineered domain bodies (SEEDbodies), Affibodies, Fynomers, Kunitz Domains, Albu-dabs, two engineered Fv fragments with exchanged VHs (e.g., a dual-affinity re-targeting molecules (D.A.R.T.s)), scFv×scFv (e.g., BiTE), DVD-IG, Covx-bodies, peptibodies, scFv-Igs, SVD-Igs, dAb-Igs, Knobs-in-Holes, IgG1 antibodies comprising matched mutations in the CH3 domain (e.g., DuoBody antibodies) and triomAbs. Exemplary bispecific formats are discussed in Garber et al., Nature Reviews Drug Discovery 13:799-801 (2014), which is herein incorporated by reference in its entirety. Additional exemplary bispecific formats are discussed in Liu et al. Front. Immunol. 8:38 doi: 10.2289/fimmu.2017.00038, and Brinkmann and Kontermann, MABS 9: 2, 182-212 (2017), each of which is herein incorporated by reference in its entirety. In certain embodiments, a bispecific antibody can be a F(ab′)2 fragment. A F(ab′)2 fragment contains the two antigen-binding arms of a tetrameric antibody molecule linked by disulfide bonds in the hinge region.

As will be appreciated by one of skill in the art, proteins and polypeptides are defined herein in terms of the amino acid sequences of the individual polypeptide chains, which are indicated by the SEQ ID NOs referenced throughout this disclosure. For example, in some embodiments an scFv-Fc-scFv protein or polypeptide described herein is comprised of two scFv-Fc-scFv polypeptide chains associated by interchain bonds (e.g., interchain disulfide bonds) to form a dimeric scFv-Fc-scFv protein (e.g., a homodimeric or heterodimeric scFv-Fc-scFv protein). In such embodiments, the scFv-Fc-scFv protein is defined by the amino acid sequences of the individual scFv-Fc-scFv polypeptide chains. Polypeptides and proteins can also comprise non-peptidic components, such as carbohydrate groups. Carbohydrates and other non-peptidic substituents can be added to a protein or polypeptide by the cell in which the protein is produced, and will vary with the type of cell. Proteins and polypeptides are defined herein in terms of their amino acid backbone structures; substituents such as carbohydrate groups are generally not specified, but may be present nonetheless.

The terms “light chain variable region” (also referred to as “light chain variable domain” or “VL” or V_{L}) and “heavy chain variable region” (also referred to as “heavy chain variable domain” or “VH” or V_{H}) refer to the variable binding region from an antibody light and heavy chain, respectively. The variable binding regions are made up of discrete, well-defined sub-regions known as “complementarity determining regions” (CDRs) and “framework regions” (FRs), generally comprising in order FR1-CDR1-FR2-CDR2-FR3-CDR3-FR4 from amino-terminus to carboxyl-terminus. In one embodiment, the FRs are humanized. The term “CL” refers to an “immunoglobulin light chain constant region” or a “light chain constant region,” i.e., a constant region from an antibody light chain. The term “CH” refers to an “immunoglobulin heavy chain constant region” or a “heavy chain constant region,” which is further divisible, depending on the antibody isotype into CH1, CH2, and CH3 (IgA, IgD, IgG), or CH1, CH2, CH3, and CH4 domains (IgE, IgM). A “Fab” (fragment antigen binding) is the part of an antibody that binds to antigens and includes the variable region and CH1 domain of the heavy chain linked to the light chain via an inter-chain disulfide bond.

The terms “amino-terminal” and “carboxyl-terminal” are used herein to denote positions within polypeptides. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl-terminus of the reference sequence, but is not necessarily at the carboxyl-terminus of the complete polypeptide.

As used herein, the term “transformation,” “transfection,” and “transduction” refer to the transfer of nucleic acid (i.e., a nucleotide polymer) into a cell. As used herein, the term “genetic transformation” refers to the transfer and incorporation of DNA, especially recombinant DNA, into a cell. The transferred nucleic acid can be introduced into a cell via an expression vector.

“Antibody-dependent cell-mediated cytotoxicity” and “ADCC,” as used herein, refer to a cell-mediated process in which nonspecific cytotoxic cells that express FcγRs (e.g., monocytic cells such as natural killer (NK) cells and macrophages) recognize bound antibody (or other protein capable of binding FcγRs) on a target cell and subsequently cause lysis of the target cell. In principle, any effector cell with an activating FcγR can be triggered to mediate ADCC. The primary cells for mediating ADCC are NK cells, which express only FcγRIII, whereas monocytes, depending on their state of activation, localization, or differentiation, can express FcγRI, FcγRII, and FcγRIII. For a review of FcγR expression on hematopoietic cells, see, e.g., Ravetch et al., 1991, Annu. Rev. Immunol., 9:457-92.

The term “having ADCC activity,” as used herein in reference to a polypeptide or protein, means that the polypeptide or protein, for example, one comprising an Fc domain (e.g., an immunoglobulin hinge region and an immunoglobulin constant region having CH2 and CH3 domains) such as derived from IgG (e.g., IgG1), is capable of mediating antibody-dependent cell-mediated cytotoxicity (ADCC) through binding of a cytolytic Fc receptor (e.g., FcγRIII) on a cytolytic immune effector cell expressing the Fc receptor (e.g., an NK cell). In some embodiments, a multispecific polypeptide or protein comprising an Fc domain may lack effector function (e.g., null ADCC activity) as the result of mutations in the CH2 and/or CH3 domain.

“Complement-dependent cytotoxicity” and “CDC,” as used herein, refer to a process in which components in normal serum (“complement”), together with an antibody or other C1q-complement-binding protein bound to a target antigen, exhibit lysis of a target cell expressing the target antigen. Complement consists of a group of serum proteins that act in concert and in an orderly sequence to exert their effect.

The terms “classical complement pathway” and “classical complement system,” as used herein, are synonymous and refer to a particular pathway for the activation of complement. The classical pathway requires antigen-antibody complexes for initiation and involves the activation, in an orderly fashion, of nine major protein components designated C1 through C9. For several steps in the activation process, the product is an enzyme that catalyzes the subsequent step. This cascade provides amplification and activation of large amounts of complement by a relatively small initial signal.

The term “having CDC activity,” as used herein in reference to a polypeptide or protein, means that the polypeptide or protein, for example, one comprising an Fc domain (e.g., an immunoglobulin hinge region and an immunoglobulin constant region having CH2 and CH3 domains) such as derived from IgG (e.g., IgG1) is capable of mediating complement-dependent cytotoxicity (CDC) through binding of C1q complement protein and activation of the classical complement system. In some embodiments, a multispecific polypeptide or protein may lack effector function (e.g., null CDC activity) as the result of one or more mutations in the CH2 and/or CH3 domains.

“Enhanced effector cell activation” as used herein, refers to the increase, prolonging, and/or potentiation of an effector cell response by the polypeptides or proteins described herein. In some embodiments, enhanced effector cell activation refers to an increase in the cytotoxic activity of an effector cell. In some embodiments, enhanced effector cell activation refers to an increase in cytokine production, cell proliferation, or a change in cell-surface molecule expression such that the ability of the effector cell to lyse a target cell is enhanced.

As used herein, the term “effector cell” refers to a cell of the immune system that is capable of lysing or killing a target cell, such as a tumor cell. Herein, an effector cell may refer to a lymphocyte, such as a T cell, a natural killer (NK) cell, or an NKT cell, a monocyte, a macrophage, a dendritic cell, or a granulocyte. In particular embodiments, the term effector cell refers to a T cell, an NK cell, or an NKT cell.

As used herein, the terms “treatment,” “treating,” or “ameliorating” refers to either a therapeutic treatment or prophylactic/preventative treatment. A treatment is therapeutic if at least one symptom of disease in an individual receiving treatment improves or a treatment can delay worsening of a progressive disease in an individual, or prevent onset of additional associated diseases.

As used herein, the term “therapeutically effective amount (or dose)” or “effective amount (or dose)” of a polypeptide or protein described herein or a composition thereof refers to that amount of the compound sufficient to result in amelioration of one or more symptoms of the disease being treated in a statistically significant manner or a statistically significant improvement in organ function. When referring to an individual active ingredient, administered alone, a therapeutically effective dose refers to that ingredient alone. When referring to a combination, a therapeutically effective dose refers to combined amounts of the active ingredients that result in the therapeutic effect, whether administered serially or simultaneously (in the same formulation or concurrently in separate formulations).

### Pharmaceutical Compositions

Described herein are stable pharmaceutical formulations of protein therapeutics, such as multispecific polypeptides, that prevent denaturation and/or prevent or substantially reduce the formation of aggregates, especially upon freezing. In addition to a therapeutic protein, the pharmaceutical compositions described herein may further comprise one or more of a buffer, an excipient, and a surfactant. In some embodiments, the compositions comprise, consist of, or consist essentially of a buffer, an excipient and a surfactant, wherein the multispecific protein is a dimer of two identical polypeptides, wherein each polypeptide comprises, in order from amino-terminus to carboxyl-terminus, or in order from carboxyl-terminus to amino-terminus (i) a first binding domain, (ii) a hinge region, (iii) an immunoglobulin constant region, and (iv) a second binding domain; and the buffer comprises or consists of succinate or a pharmaceutically acceptable salt or acid thereof.

In some embodiments, the composition comprises from about 0.1 mg/ml to about 10 mg/ml of the multispecific protein. In some embodiments, the composition comprises from about 1 mg/ml to about 5 mg/ml of the multispecific protein. In some embodiments, the composition comprises about 2 mg/ml of the multispecific protein. In some embodiments, the composition comprises about 2 mg/ml of the multispecific protein, about 5 mM succinate, about 6.5% weight/volume (w/v) sucrose and about 0.02% w/v polysorbate 80.

In some embodiments, wherein the composition substantially prevents degradation of the multispecific protein. In some embodiments, the composition slows or reduces the degradation of the multispecific polypeptide as compared to an identical multispecific polypeptide stored in histidine buffer under identical storage conditions. In some embodiments, the composition is substantially stable for at least 1 year at 4° C. In some embodiments, the composition is substantially resistant to formation of aggregates of multispecific protein.

In some embodiments, the composition is capable of withstanding freeze to thaw conditions. In some embodiments, the composition slows or reduces degradation of the multispecific polypeptide in freeze to thaw conditions as compared to a multispecific polypeptide stored in a histidine buffer under identical freeze to thaw conditions.

In other embodiments, a CD123×CD3 targeting multispecific polypeptide undergoes little to no degradation after lyophilization when formulated as disclosed herein. For instance, a CD123×CD3 targeting multispecific polypeptide may be formulated in a succinate and sucrose formulation that exhibits reduced degradation after lyophilization as compared to an identical polypeptide formulated with a histidine buffer. Also provided herein is a lyophilized anti-CD123×anti-CD3 multispecific polypeptide, including but not limited to TRI130 and TRI129, formulated in about 5 mM succinate, about 6.5% weight/volume (w/v) sucrose and about 0.02% w/v polysorbate 80. In some embodiments, the composition is lyophilized.

### Buffer

As used herein, the term “buffer” or “buffering agent” refers to one or more components that when added to an aqueous solution is able to protect the solution against variations in pH when adding acid or alkali, or upon dilution with a solvent.

In some embodiments, the buffer comprises, consists of, or consists essentially of any pharmaceutically acceptable buffer. For example, the buffer may be potassium phosphate, acetic acid/sodium acetate, citric acid/sodium citrate, succinic acid/sodium succinate, tartaric acid/sodium tartrate, histidine/histidine HCl, glycine, Tris, glutamate, acetate, mixtures thereof, or pharmaceutically acceptable salts or acids thereof. In particular embodiments, the buffer comprises, consists of, or consists essentially of succinate or a pharmaceutically acceptable salt or acid thereof.

In some embodiments, the concentration of the buffer in the composition is from about 1 mM to about 500 mM, from about 1 mM to about 100 mM, from about 1 mM to about 50 mM, from about 1 to about 10 mM, from about 5 mM to about 50 mM, or from about 5 mM to about 20 mM from about 5 mM to about 10 mM. In some embodiments, the composition comprises from about 1 mM to about 10 mM succinate or a pharmaceutically acceptable salt or acid thereof. In some embodiments, the composition comprises about 5 mM succinate or a pharmaceutically acceptable salt or acid thereof.

In some embodiments, the pH of the composition is 3.0, 3.25, 3.5, 3.75, 4.0, 4.25, 4.5, 4.75, 5.0, 5.25, 5.5, 5.75, 6.0, 6.25, 6.5, 6.75, 7.0, 7.25, 7.5, 7.75, 8.0, 8.25, 8.5, 8.75, 9.0, 9.25, 9.5, 9.75, 10.0, 10.25, 10.5, 10.75, 11.0, 11.25 or 11.5. In some embodiments, the pH of the composition is about 3.0 to about 6.0. In some embodiments, the composition has a pH from about 4.0 to about 5.5. In some embodiments, the pH of the composition is about 4.8.

### Excipient

As referred to herein, an excipient is a pharmacologically inactive substance formulated alongside the active pharmaceutical ingredient of a composition. Excipients might aid in lubricity, flowability, disintegration, or taste and may confer some form of antimicrobial function.

Exemplary excipients which may be used in the compositions disclosed herein include pharmaceutical binders, diluents, release retarding excipients, lubricant, glidants, gas generating agents, coating systems, solvents, and coloring agents. Suitable excipients include the substances mentioned as excipients in the Handbook of Pharmaceutical Excipients, Third Edition, Edited by A. H. Kibbe, American Pharmaceutical Association and Pharmaceutical Press (2000), and Tables 3-5 in E. T. Cole et al., Advanced Drug Delivery Reviews 60 (2008), 747-756. For example, an excipient may be selected from the group consisting of polypropylene glycol; polyethylene glycol, polyoxyethylene castor oil derivatives, polyoxyethylene glycerol oxystearate, saturated polyglycolized glycerides, polyethylene polypropylene glycol, Vitamin E, and Vitamin E TPGS (d-alpha—tocopheryl polyethylene glycol 1000 succinate).

In some embodiments, the composition comprises from about 1% weight/volume (w/v) to about 20% w/v, about 1% w/v to about 10% w/v, about 5% w/v to about 15% w/v, or about 10% w/v of the excipient. In some embodiments, the composition comprises from about 1% w/v to about 12% w/v of the excipient, such as about 6.5% w/v of the excipient.

In some embodiments, the excipient the excipient comprises, consists of, or consists essentially of a sugar. In some embodiments, the composition comprises from about 1% w/v to about 12% w/v of the sugar. In some embodiments, the composition comprises about 4% to about 8% w/v of the sugar. In some embodiments, the composition comprises about 6.5% w/v of the sugar. In some embodiments, the sugar is sucrose.

### Surfactant

As described herein, a “surfactant” is a surface active molecule containing both a hydrophobic portion (e.g., alkyl chain) and a hydrophilic portion (e.g., carboxyl and carboxylate groups).

Surfactants suitable for use in the compositions described herein include, but are not limited to, polysorbates (e.g. polysorbates 20 or 80); poloxamers (e.g. poloxamer 188); sorbitan esters and derivatives; Triton; sodium laurel sulfate; sodium octyl glycoside; lauryl-, myristyl-, linoleyl-, or stearyl-sulfobetadine; lauryl-, myristyl-, linoleyl- or stearyl-sarcosine; linoleyl-, myristyl-, or cetyl-betaine; lauramidopropyl-cocamidopropyl-, linoleamidopropyl-, myristamidopropyl-, palmidopropyl-, or isostearamidopropylbetaine (e.g., lauroamidopropyl); myristamidopropyl-, palmidopropyl-, or isostearamidopropyl-dimethylamine; sodium methyl cocoyl-, or disodium methyl oleyl-taurate); and the MONAQUAT™ series (Mona Industries, Inc., Paterson, N.J.), polyethylene glycol, polypropyl glycol, and copolymers of ethylene and propylene glycol (e.g., Pluronics, PF68 etc.). In particular embodiments, the surfactant comprises or consists of polysorbate 80.

In some embodiments, the composition comprises from about 0.001% w/v to about 1% w/v, about 0.01% w/v to about 0.5% w/v, or about 0.01% w/v to about 0.1% w/v of the surfactant. In some embodiments, the composition comprises about 0.02% w/v of the surfactant.

In some embodiments, the composition comprises from about 0.001% w/v to about 1% w/v, about 0.01% w/v to about 0.5% w/v, or about 0.01% w/v to about 0.1% w/v of polysorbate 80. In some embodiments, the composition comprises about 0.02% w/v of polysorbate 80.

### Therapeutic Proteins

The compositions described herein may be used in connection with many different protein therapeutics as described herein.

### Binding Domains

In some embodiments, the therapeutic proteins comprise a binding domain. The binding domain may provide for specific binding to at least one cell-surface molecule (e.g., a cell-surface receptor). The binding domain can be in the form of an antibody, or fragment thereof, or a fusion protein of any of a variety of different formats (e.g., the fusion protein can be in the form of a bispecific or multispecific molecule). In other embodiments, the binding domain can comprise, for example, a particular cytokine or a molecule that targets the binding domain polypeptide to, for example, a particular cell type, a toxin, an additional cell receptor, or an antibody.

In some embodiments, a binding domain described herein is derived from an antibody and comprises a variable heavy chain (V_{H}) and a variable light chain (V_{L}). For example, an scFv comprising a V_{H} and V_{L} chain. These binding domains and variable chains may be arranged in any order that still retains some binding to the target(s). In some embodiments, a binding domain comprises (i) an immunoglobulin heavy chain variable region (V_{H}) comprising HCDR1, HCDR2, and HCDR3; and (ii) an immunoglobulin light chain variable region (V_{L}) comprising LCDR1, LCDR2, and LCDR3.

In some embodiments, the polypeptides and proteins described herein comprise binding domains that are scFvs. In such embodiments, the binding domains may be referred to as scFv domains. In some embodiments, a binding domain is a single-chain Fv fragment (scFv) that comprises V_{H} and V_{L} regions specific for a target of interest. In certain embodiments, the V_{H} and V_{L} regions are human or humanized. In some variations, a binding domain is a single-chain Fv (scFv) comprising V_{L} and V_{H} regions joined by a peptide linker.

In certain embodiments, the binding domains of the polypeptides described herein comprise (i) an immunoglobulin light chain variable region (V_{L}) comprising CDRs LCDR1, LCDR2, and LCDR3, and (ii) an immunoglobulin heavy chain variable region (V_{H}) comprising CDRs HCDR1, HCDR2, and HCDR3. In some embodiments, amino acid sequences provided for polypeptide constructs do not include the human immunoglobulin leader sequences. CDR sequences and amino acid substitution positions shown are those defined using the IMGT criteria (Brochet et al, Nucl. Acids Res. (2008) 36, W503-508).

In certain embodiments, a binding domain V_{L} and/or V_{H} region of the present disclosure is derived from a V_{L} and/or V_{H} of a parent V_{L} and/or V_{H} region (e.g., 1618/1619 as described in PCT Application Publication No. WO 2016/185016) and optionally contains about one or more (e.g., about 2, 3, 4, 5, 6, 7, 8, 9, 10) insertions, about one or more (e.g., about 2, 3, 4, 5, 6, 7, 8, 9, 10) deletions, about one or more (e.g., about 2, 3, 4, 5, 6, 7, 8, 9, 10) amino acid substitutions (e.g., conservative amino acid substitutions or non-conservative amino acid substitutions), or a combination of the above-noted changes, when compared to the V_{L} and/or V_{H} sequence of a known monoclonal antibody. The insertion(s), deletion(s) or substitution(s) can be anywhere in the V_{L} and/or V_{H} region, including at the amino- or carboxyl-terminus or both ends of this region, provided that each CDR comprises zero changes or at most one, two, or three changes. In some embodiments, the binding domain containing the modified V_{L} and/or V_{H} region can still specifically bind its target with an affinity similar to or greater than the parent binding domain.

The use of peptide linkers for joining V_{L} and V_{H} regions is well-known in the art, and a large number of publications exist within this particular field. In some embodiments, a peptide linker is a 15mer consisting of three repeats of a Gly-Gly-Gly-Gly-Ser (SEQ ID NO: 128) amino acid sequence ((Gly₄Ser)₃) (SEQ ID NO: 59). Other linkers have been used, and phage display technology, as well as selective infective phage technology, has been used to diversify and select appropriate linker sequences (Tang et al., J. Biol. Chem. 271, 15682-15686, 1996; Hennecke et al., Protein Eng. 11, 405-410, 1998). In certain embodiments, the V_{L} and V_{H} regions are joined by a peptide linker having an amino acid sequence comprising the formula (Gly₄Ser)ₙ, wherein n=1-5 (SEQ ID NO: 129). For instance, in some embodiments, the linker comprises (Gly4Ser)₄ (SEQ ID NO: 61). Other suitable linkers can be obtained by optimizing a simple linker through random mutagenesis. In some embodiments, the V_{H} region of the scFv described herein may be positioned N-terminally to a linker sequence. In some embodiments, the V_{L} region of the scFvs described herein may be positioned C-terminally to the linker sequence.

In some embodiments, the binding domain may bind to a tumor antigen, such as CD123, PSMA, CD19, CD33, 5T4, or HER2. In some embodiments, the binding domain may be a CD3 binding domain. In some embodiments, the binding domain may bind to 4-1-BB. In some embodiments, the binding domain may bind to OX40. In some embodiments, a formulated multispecific protein binds to both 4-1 BB and OX40.

### Hinge

In addition to a binding domain, the therapeutic polypeptides may further comprise a hinge region. In some embodiments, the hinge is an altered immunoglobulin hinge in which one or more cysteine residues in a wild type immunoglobulin hinge region are substituted with one or more other amino acid residues (e.g., serine or alanine). Exemplary altered immunoglobulin hinges, carboxyl-terminus linkers, and amino-terminus linkers include an immunoglobulin human IgG1 hinge region having one, two or three cysteine residues found in a wild type human IgG1 hinge substituted by one, two or three different amino acid residues (e.g., serine or alanine). An altered immunoglobulin hinge can additionally have a proline substituted with another amino acid (e.g., serine or alanine). For example, the above-described altered human IgG1 hinge can additionally have a proline located carboxyl-terminal to the three cysteines of wild type human IgG1 hinge region substituted by another amino acid residue (e.g., serine, alanine). In one embodiment, the prolines of the core hinge region are not substituted. In certain embodiments, a hinge, a carboxyl-terminus linker, or an amino-terminus linker polypeptide comprises or is a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to a wild type immunoglobulin hinge region, such as a wild type human IgG1 hinge, a wild type human IgG2 hinge, or a wild type human IgG4 hinge.

### Immunoglobulin Constant Domain

The therapeutic proteins may also comprise an immunoglobulin constant (Fc) domain (also referred to herein as a constant region, Fc domain, Fc region, and the like). In come embodiments, the constant region comprises IgG CH2 and CH3 domains, e.g., IgG1 CH2 and CH3 domains. In some embodiments, the constant region does not comprise a CH1 domain. In some embodiments, the immunoglobulin constant region is a human Fc domain. In some embodiments, the immunoglobulin constant region comprises one, two, three or more amino acid substitutions compared to a wild-type immunoglobulin constant region to reduce or prevent binding to FcγR1, FcγRIIa, FcγRIIb, FcγRIIa, and FcγRIIIb. In some embodiments, the constant domains making up the constant region are human or derived from human sequences. In some embodiments, the Fc domain comprises one or more mutations the Fc region to reduce or prevent complement fixation and interaction with Fcγ receptors. In some embodiments, the immunoglobulin constant region comprises one, two, three or more amino acid substitutions compared to a wild-type immunoglobulin constant region to prevent or reduce Fc-mediated T-cell activation. In some embodiments, the immunoglobulin constant region comprises one, two, three or more amino acid substitutions compared to a wild-type immunoglobulin constant region to prevent or reduce CDC activity. In some embodiments, the immunoglobulin constant region comprises one, two, three or more amino acid substitutions compared to a wild-type immunoglobulin constant region to prevent or reduce ADCC activity.

In some embodiments, the Fc region comprises one or more mutations at positions 234, 235, 237 and 322 of the CH2 domain, according to the EU numbering system. In some embodiments, the Fc domain comprises mutations at positions 234, 235, 237, 318, 320 and 322 of the CH2 domain, according to the EU numbering system. In some embodiments, the Fc domain comprises mutations L234A, L235A, G237A and K322A of the CH2 domain, according to the EU numbering system. In some embodiments, the Fc domain comprises mutations L234A, L235A, G237A, E318A, K320A, and K322A of the CH2 domain, according to the EU numbering system. In some embodiments, the immunoglobulin constant region comprises a human IgG1 CH2 domain comprising the substitutions E233P, L234A, L235A, G237A, and K322A and a deletion of G236, according to the EU numbering system. In some embodiments, the Fc domain is derived from human IgG1. In some embodiments, the two or more mutations in the IgG1 Fc domain prevent or substantially reduce signaling through Fc-mediated cross-linking.

In some embodiments, the immunoglobulin constant region comprises an amino acid sequence of any one of SEQ ID NO:32-35, or a variant thereof. The inclusion of an immunoglobulin constant region slows clearance of the polypeptides and proteins of the present disclosure from circulation after administration to a subject. By mutations or other alterations, an immunoglobulin constant region further enables relatively easy modulation of polypeptide effector functions (e.g., ADCC, ADCP, CDC, complement fixation, and binding to Fc receptors), which can either be increased or decreased depending on the disease being treated, as known in the art and described herein. In certain embodiments, the polypeptides and proteins described herein comprise an immunoglobulin constant region capable of mediating one or more of these effector functions. In other embodiments, one or more of these effector functions are reduced or absent in an immunoglobulin constant region of a polypeptide or protein described in the present disclosure, as compared to a corresponding wild-type immunoglobulin constant region.

An immunoglobulin constant region present in the polypeptides and proteins of the present disclosure can comprise or can be derived from part or all of: a CH2 domain, a CH3 domain, a CH4 domain, or any combination thereof. For example, an immunoglobulin constant region can comprise a CH2 domain, a CH3 domain, both CH2 and CH3 domains, both CH3 and CH4 domains, two CH3 domains, a CH4 domain, two CH4 domains, and a CH2 domain and part of a CH3 domain. In certain embodiments, the polypeptides or proteins described herein do not comprise a CH1 domain.

A polypeptide or protein described herein may comprise a wild type immunoglobulin CH2 domain or an altered immunoglobulin CH2 domain from certain immunoglobulin classes or subclasses (e.g., IgG1, IgG2, IgG3, IgG4, IgA1, IgA2, or IgD) and from various species (including human, mouse, rat, and other mammals). In certain embodiments, a CH2 domain of a polypeptide or a protein described herein is a wild type human immunoglobulin CH2 domain, such as wild type CH2 domains of human IgG1, IgG2, IgG3, IgG4, IgA1, IgA2, or IgD, as set forth in SEQ ID NOs: 115, 199-201 and 195-197, respectively, of U.S. Patent Application Publication No. 2013/0129723 (said sequences incorporated by reference herein). In certain embodiments, the CH2 domain is a wild type human IgG1 CH2 domain as set forth in SEQ ID NO: 115 of U.S. Patent Application Publication No. US 2013/0129723 (said sequence incorporated by reference herein).

In certain embodiments, an altered CH2 region in a polypeptide or a protein of the present disclosure comprises or is a sequence that is at least 90%, at least 91%, at least 92%, at least 93%, at least 94%, at least 95%, at least 96%, at least 97%, at least 98%, at least 99% identical to a wild type immunoglobulin CH2 region, such as the CH2 region of wild type human IgG1, IgG2, or IgG4, or mouse IgG2a (e.g., IGHG2c).

An altered immunoglobulin CH2 region in a polypeptide or protein of the present disclosure can be derived from a CH2 region of various immunoglobulin isotypes, such as IgG1, IgG2, IgG3, IgG4, IgA1, IgA2, and IgD, from various species (including human, mouse, rat, and other mammals). In certain embodiments, an altered immunoglobulin CH2 region in a fusion protein of the present disclosure can be derived from a CH2 region of human IgG1, IgG2 or IgG4, or mouse IgG2a (e.g., IGHG2c), whose sequences are set forth in SEQ ID NOs: 115, 199, 201, and 320 of U.S. Patent Application Publication No. 2013/0129723 (said sequences incorporated by reference herein). In certain embodiments, an altered CH2 domain of a polypeptide or a protein described herein is an altered human IgG1 CH2 domain with mutations known in the art that enhance or reduce immunological activities (i.e., effector functions) such as ADCC, ADCP, CDC, complement fixation, Fc receptor binding, or any combination thereof.

In certain embodiments, a CH2 domain of a polypeptide or a protein described herein is an altered immunoglobulin CH2 region (e.g., an altered human IgG1 CH2 domain) that comprises one or more amino acid deletions or substitutions. In some embodiments, the CH2 domain comprises an amino acid substitution at the asparagine of position 297 (e.g., asparagine to alanine). Such an amino acid substitution reduces or eliminates glycosylation at this site and abrogates efficient Fc binding to FcγR and C1q. The sequence of an altered human IgG1 CH2 domain with an Asn to Ala substitution at position 297 is set forth in SEQ ID NO: 324 of U.S. Patent Application Publication No. 2013/0129723 (said sequence incorporated by reference herein). In some embodiments, the altered CH2 domain comprises at least one substitution or deletion at positions 234 to 238. For example, an immunoglobulin CH2 region can comprise a substitution at position 234, 235, 236, 237 or 238; positions 234 and 235; positions 234 and 236; positions 234 and 237; positions 234 and 238; positions 234-236; positions 234, 235 and 237; positions 234, 236 and 238; positions 234, 235, 237, and 238; positions 236-238; or any other combination of two, three, four, or five amino acids at positions 234-238. In some embodiments, an altered CH2 region comprises one or more (e.g., two, three, four or five) amino acid deletions at positions 234-238, for instance, at one of position 236 or position 237 while the other position is substituted. In certain embodiments, the amino acid residues at one or more of positions 234-238 has been replaced with one or more alanine residues. In further embodiments, only one of the amino acid residues at positions 234-238 have been deleted while one or more of the remaining amino acids at positions 234-238 can be substituted with another amino acid (e.g., alanine or serine).

In some embodiments, the above-noted mutation(s) decrease or eliminate the ADCC activity or Fc receptor-binding capability of a polypeptide that comprises the altered CH2 domain.

In certain other embodiments, a CH2 domain of a polypeptide or a protein described herein is an altered immunoglobulin CH2 region (e.g., an altered human IgG1 CH2 domain) that comprises one or more amino acid substitutions at positions 253, 310, 318, 320, 322, and 331. For example, an immunoglobulin CH2 region can comprise a substitution at position 253, 310, 318, 320, 322, or 331, positions 318 and 320, positions 318 and 322, positions 318, 320 and 322, or any other combination of two, three, four, five or six amino acids at positions 253, 310, 318, 320, 322, and 331. In such embodiments, the above-noted mutation(s) decrease or eliminate the CDC activity of a polypeptide comprising the altered CH2 domain.

In certain other embodiments, in addition to the amino acid substitution at position 297, an altered CH2 region of a polypeptide or a protein described herein (e.g., an altered human IgG1 CH2 domain) can further comprise one or more (e.g., two, three, four, or five) additional substitutions at positions 234-238. For example, an immunoglobulin CH2 region can comprise a substitution at positions 234 and 297, positions 234, 235, and 297, positions 234, 236 and 297, positions 234-236 and 297, positions 234, 235, 237 and 297, positions 234, 236, 238 and 297, positions 234, 235, 237, 238 and 297, positions 236-238 and 297, or any combination of two, three, four, or five amino acids at positions 234-238 in addition to position 297. In addition or alternatively, an altered CH2 region can comprise one or more (e.g., two, three, four or five) amino acid deletions at positions 234-238, such as at position 236 or position 237. The additional mutation(s) decreases or eliminates the ADCC activity or Fc receptor-binding capability of a polypeptide comprising the altered CH2 domain. In certain embodiments, the amino acid residues at one or more of positions 234-238 have been replaced with one or more alanine residues. In further embodiments, only one of the amino acid residues at positions 234-238 has been deleted while one or more of the remaining amino acids at positions 234-238 can be substituted with another amino acid (e.g., alanine or serine).

In certain embodiments, in addition to one or more (e.g., 2, 3, 4, or 5) amino acid substitutions at positions 234-238, a mutated CH2 region of a polypeptide or a protein described herein (e.g., an altered human IgG1 CH2 domain) in a fusion protein of the present disclosure can contain one or more (e.g., 2, 3, 4, 5, or 6) additional amino acid substitutions (e.g., substituted with alanine) at one or more positions involved in complement fixation (e.g., at positions 1253, H310, E318, K320, K322, or P331). Examples of mutated immunoglobulin CH2 regions include human IgG1, IgG2, IgG4 and mouse IgG2a CH2 regions with alanine substitutions at positions 234, 235, 237 (if present), 318, 320 and 322. An exemplary mutated immunoglobulin CH2 region is mouse IGHG2c CH2 region with alanine substitutions at L234, L235, G237, E318, K320, and K322.

In still further embodiments, in addition to the amino acid substitution at position 297 and the additional deletion(s) or substitution(s) at positions 234-238, an altered CH2 region of a polypeptide or a protein described herein (e.g., an altered human IgG1 CH2 domain) can further comprise one or more (e.g., two, three, four, five, or six) additional substitutions at positions 253, 310, 318, 320, 322, and 331. For example, an immunoglobulin CH2 region can comprise a (1) substitution at position 297, (2) one or more substitutions or deletions or a combination thereof at positions 234-238, and one or more (e.g., 2, 3, 4, 5, or 6) amino acid substitutions at positions 1253, H310, E318, K320, K322, and P331, such as one, two, three substitutions at positions E318, K320 and K322. The amino acids at the above-noted positions can be substituted by alanine or serine.

In certain embodiments, an immunoglobulin CH2 region of a polypeptide or a protein described herein comprises: (i) an amino acid substitution at the asparagines of position 297 and one amino acid substitution at position 234, 235, 236 or 237; (ii) an amino acid substitution at the asparagine of position 297 and amino acid substitutions at two of positions 234-237; (iii) an amino acid substitution at the asparagine of position 297 and amino acid substitutions at three of positions 234-237; (iv) an amino acid substitution at the asparagine of position 297, amino acid substitutions at positions 234, 235 and 237, and an amino acid deletion at position 236; (v) amino acid substitutions at three of positions 234-237 and amino acid substitutions at positions 318, 320 and 322; or (vi) amino acid substitutions at three of positions 234-237, an amino acid deletion at position 236, and amino acid substitutions at positions 318, 320 and 322.

Exemplary altered immunoglobulin CH2 regions with amino acid substitutions at the asparagine of position 297 include: human IgG1 CH2 region with alanine substitutions at L234, L235, G237 and N297 and a deletion at G236 (SEQ ID NO: 325 of U.S. Patent Application Publication No. 2013/0129723, said sequence incorporated by reference herein), human IgG2 CH2 region with alanine substitutions at V234, G236, and N297 (SEQ ID NO: 326 of U.S. Patent Application Publication No. 2013/0129723, said sequence incorporated by reference herein), human IgG4 CH2 region with alanine substitutions at F234, L235, G237 and N297 and a deletion of G236 (SEQ ID NO: 322 of U.S. Patent Application Publication No. 2013/0129723, said sequence incorporated by reference herein), human IgG4 CH2 region with alanine substitutions at F234 and N297 (SEQ ID NO: 343 of U.S. Patent Application Publication No. US 2013/0129723, said sequence incorporated by reference herein), human IgG4 CH2 region with alanine substitutions at L235 and N297 (SEQ ID NO: 344 of U.S. Patent Application Publication No. 2013/0129723, said sequence incorporated by reference herein), human IgG4 CH2 region with alanine substitutions at G236 and N297 (SEQ ID NO: 345 of U.S. Patent Application Publication No. 2013/0129723, said sequence incorporated by reference herein), and human IgG4 CH2 region with alanine substitutions at G237 and N297 (SEQ ID NO: 346 of U.S. Patent Application Publication No. 2013/0129723, said sequence incorporated by reference herein). These CH2 regions can be used in a polypeptide of the present disclosure.

In certain embodiments, in addition to the amino acid substitutions described above, an altered CH2 region of a polypeptide or a protein described herein (e.g., an altered human IgG1 CH2 domain) can contain one or more additional amino acid substitutions at one or more positions other than the above-noted positions. Such amino acid substitutions can be conservative or non-conservative amino acid substitutions. For example, in certain embodiments, P233 can be changed to E233 in an altered IgG2 CH2 region (see, e.g., SEQ ID NO: 326 of U.S. Patent Application Publication No. 2013/0129723, said sequence incorporated by reference herein). In addition or alternatively, in certain embodiments, the altered CH2 region can contain one or more amino acid insertions, deletions, or both. The insertion(s), deletion(s) or substitution(s) can be anywhere in an immunoglobulin CH2 region, such as at the N- or C-terminus of a wild type immunoglobulin CH2 region resulting from linking the CH2 region with another region (e.g., a binding domain or an immunoglobulin heterodimerization domain) via a hinge.

In certain embodiments, an altered CH2 domain of a polypeptide or protein described herein is a human IgG1 CH2 domain with alanine substitutions at positions 235, 318, 320, and 322 (i.e., a human IgG1 CH2 domain with L235A, E318A, K320A and K322A substitutions) (SEQ ID NO: 595 of U.S. Patent Application Publication No. 2013/0129723, said sequence incorporated by reference herein), and optionally an N297 mutation (e.g., to alanine). In certain other embodiments, an altered CH2 domain is a human IgG1 CH2 domain with alanine substitutions at positions 234, 235, 237, 318, 320 and 322 (i.e., a human IgG1 CH2 domain with L234A, L235A, G237A, E318A, K320A and K322A substitutions) (SEQ ID NO: 596 of U.S. Patent Application Publication No. 2013/0129723, said sequence incorporated by reference herein), and optionally an N297 mutation (e.g., to alanine).

In some embodiments, an immunoglobulin constant region of a polypeptide or a protein described herein comprises a human IgG1 CH2 domain comprising the substitutions L234A, L235A, G237A, and K322A, according to the EU numbering system.

The CH3 domain that can form an immunoglobulin constant region of a polypeptide or a protein described herein can be a wild type immunoglobulin CH3 domain or an altered immunoglobulin CH3 domain thereof from certain immunoglobulin classes or subclasses (e.g., IgG1, IgG2, IgG3, IgG4, IgA1, IgA2, IgD, IgE, IgM) of various species (including human, mouse, rat, and other mammals). In certain embodiments, a CH3 domain of a polypeptide described herein is a wild type human immunoglobulin CH3 domain, such as wild type CH3 domains of human IgG1, IgG2, IgG3, IgG4, IgA1, IgA2, IgD, IgE, or IgM as set forth in SEQ ID NOs: 116, 208-210, 204-207, and 212, respectively of U.S. Patent Application Publication No. 2013/0129723 (said sequences incorporated by reference herein). In certain embodiments, the CH3 domain is a wild type human IgG1 CH3 domain as set forth in SEQ ID NO: 116 of U.S. Patent Application Publication No. 2013/0129723 (said sequence incorporated by reference herein).

In certain embodiments, a CH3 domain of a polypeptide described herein is an altered human immunoglobulin CH3 domain, such as an altered CH3 domain based on or derived from a wild-type CH3 domain of human IgG1, IgG2, IgG3, IgG4, IgA1, IgA2, IgD, IgE, or IgM antibodies. For example, an altered CH3 domain can be a human IgG1 CH3 domain with one or two mutations at positions H433 and N434 (positions are numbered according to EU numbering). The mutations in such positions can be involved in complement fixation. In certain other embodiments, an altered CH3 domain of a polypeptide described herein can be a human IgG1 CH3 domain but with one or two amino acid substitutions at position F405 or Y407. The amino acids at such positions are involved in interacting with another CH3 domain. In certain embodiments, an altered CH3 domain of polypeptide described herein can be an altered human IgG1 CH3 domain with its last lysine deleted. The sequence of this altered CH3 domain is set forth in SEQ ID NO: 761 of U.S. Patent Application Publication No. 2013/0129723 (said sequence incorporated by reference herein).

In certain embodiments, a polypeptide or a protein described herein comprises a CH3 domain that comprises so called “knobs-into-holes” mutations (see, Marvin and Zhu, Acta Pharmacologica Sinica 26:649-58, 2005; Ridgway et al., Protein Engineering 9:617-21, 1966). More specifically, mutations can be introduced into each of the CH3 domains of each polypeptide chain so that the steric complementarity required for CH3/CH3 association obligates these two CH3 domains to pair with each other. For example, a CH3 domain in one single chain polypeptide of a polypeptide heterodimer can contain a T366W mutation (a “knob” mutation, which substitutes a small amino acid with a larger one), and a CH3 domain in the other single chain polypeptide of the polypeptide heterodimer can contain a Y407A mutation (a “hole” mutation, which substitutes a large amino acid with a smaller one). Other exemplary knobs-into-holes mutations include (1) a T366Y mutation in one CH3 domain and a Y407T in the other CH3 domain, and (2) a T366W mutation in one CH3 domain and T366S, L368A and Y407V mutations in the other CH3 domain.

The CH4 domain that can form an immunoglobulin constant region a polypeptide or a protein described herein can be a wild type immunoglobulin CH4 domain or an altered immunoglobulin CH4 domain thereof from IgE or IgM molecules. In certain embodiments, the CH4 domain of a polypeptide described herein is a wild type human immunoglobulin CH4 domain, such as wild type CH4 domains of human IgE and IgM molecules as set forth in SEQ ID NOs: 213 and 214, respectively, of U.S. Patent Application Publication No. 2013/0129723 (said sequences incorporated by reference herein). In certain embodiments, a CH4 domain of a polypeptide described herein is an altered human immunoglobulin CH4 domain, such as an altered CH4 domain based on or derived from a CH4 domain of human IgE or IgM molecules, which have mutations that increase or decrease an immunological activity known to be associated with an IgE or IgM Fc region.

In certain embodiments, an immunoglobulin constant region of a polypeptide or a protein described herein comprises a combination of CH2, CH3 or CH4 domains (i.e., more than one constant region domain selected from CH2, CH3 and CH4). For example, the immunoglobulin constant region can comprise CH2 and CH3 domains or CH3 and CH4 domains. In certain other embodiments, the immunoglobulin constant region can comprise two CH3 domains and no CH2 or CH4 domains (i.e., only two or more CH3). The multiple constant region domains that form an immunoglobulin constant region of the polypeptides described herein can be based on or derived from the same immunoglobulin molecule, or the same class or subclass immunoglobulin molecules. In certain embodiments, the immunoglobulin constant region is an IgG CH2-CH3 (e.g., IgG1 CH2-CH3, IgG2 CH2-CH3, and IgG4 CH2-CH3) and can be a human (e.g., human IgG1, IgG2, and IgG4) CH2CH3. For example, in certain embodiments, the immunoglobulin constant region of a polypeptide described herein comprises (1) wild type human IgG1 CH2 and CH3 domains, (2) human IgG1 CH2 with N297A substitution (i.e., CH2(N297A)) and wild type human IgG1 CH3, or (3) human IgG1 CH2(N297A) and an altered human IgG1 CH3 with the last lysine deleted. Alternatively, the multiple constant region domains of a polypeptide or a protein described herein can be based on or derived from different immunoglobulin molecules, or different classes or subclasses immunoglobulin molecules. For example, in certain embodiments, an immunoglobulin constant region comprises both human IgM CH3 domain and human IgG1 CH3 domain. The multiple constant region domains that form an immunoglobulin constant region of a polypeptide described herein can be directly linked together or can be linked to each other via one or more (e.g., about 2-10) amino acids.

Exemplary immunoglobulin constant regions that can be used in a polypeptide or a protein described herein are set forth in SEQ ID NOs: 305-309, 321, 323, 341, 342, and 762 of U.S. Patent Application Publication No. 2013/0129723 (said sequences incorporated by reference herein). Further exemplary immunoglobulin constant regions that can be used in a polypeptide or a protein described herein are provided in the table below.

TABLE 1
Exemplary immunoglobulin constant regions

| | | DNA | | AA |
|---|---|---|---|---|
| | | SEQ | | SEQ |
| | | ID | | ID |
| Name | DNA Sequence | NO: | AA Sequence | NO: |
| SS-Fc | TCGAGTGAGCCCAAATCTTCT | 32 | SSEPKSSDKTHT | 33 |
| domain | GACAAAACTCACACATGCCCA | | CPPCPAPEAAG | |
| | CCGTGCCCAGCACCTGAAGC | | APSVFLFPPKPK | |
| | CGCGGGTGCACCGTCAGTCTT | | DTLMISRTPEVT | |
| | CCTCTTCCCCCCAAAACCCAA | | CVVVDVSHEDP | |
| | GGACACCCTCATGATCTCCCG | | EVKFNWYVDGV | |
| | GACCCCTGAGGTCACATGCGT | | EVHNAKTKPRE | |
| | GGTGGTGGACGTGAGCCACG | | EQYNSTYRVVS | |
| | AAGACCCTGAGGTCAAGTTCA | | VLTVLHQDWLN | |
| | ACTGGTACGTGGACGGCGTG | | GKEYKCAVSNK | |
| | GAGGTGCATAATGCCAAGACA | | ALPAPIEKTISKA | |
| | AAGCCGCGGGAGGAGCAGTA | | KGQPREPQVYT | |
| | CAACAGCACGTACCGTGTGGT | | LPPSRDELTKNQ | |
| | CAGCGTCCTCACCGTCCTGCA | | VSLTCLVKGFYP | |
| | CCAGGACTGGCTGAATGGCAA | | SDIAVEWESNG | |
| | GGAATACAAGTGCGCGGTCTC | | QPENNYKTTPP | |
| | CAACAAAGCCCTCCCAGCCCC | | VLDSDGSFFLYS | |
| | CATCGAGAAAACCATCTCCAA | | KLTVDKSRWQQ | |
| | AGCCAAAGGGCAGCCCCGAG | | GNVFSCSVMHE | |
| | AACCACAGGTGTACACCCTGC | | ALHNHYTQKSLS | |
| | CCCCATCCCGGGATGAGCTGA | | LSPG | |
| | CCAAGAACCAGGTCAGCCTGA | | | |
| | CCTGCCTGGTCAAAGGCTTCT | | | |
| | ATCCAAGCGACATCGCCGTGG | | | |
| | AGTGGGAGAGCAATGGGCAG | | | |
| | CCGGAGAACAACTACAAGACC | | | |
| | ACGCCTCCCGTGCTGGACTCC | | | |
| | GACGGCTCCTTCTTCCTCTAC | | | |
| | AGCAAGCTCACCGTGGACAAG | | | |
| | AGCAGGTGGCAGCAGGGGAA | | | |
| | CGTCTTCTCATGCTCCGTGAT | | | |
| | GCATGAGGCTCTGCACAACCA | | | |
| | CTACACGCAGAAGAGCCTCTC | | | |
| | CCTGTCTCCGGGT | | | |
| Delta | GAGCCCAAATCTTCTGACAAA | 34 | EPKSSDKTHTCP | 35 |
| SS-Fc | ACTCACACATGCCCACCGTGC | | PCPAPEAAGAP | |
| domain | CCAGCACCTGAAGCCGCGGG | | SVFLFPPKPKDT | |
| | TGCACCGTCAGTCTTCCTCTT | | LMISRTPEVTCV | |
| | CCCCCCAAAACCCAAGGACAC | | VVDVSHEDPEV | |
| | CCTCATGATCTCCCGGACCCC | | KFNWYVDGVEV | |
| | TGAGGTCACATGCGTGGTGGT | | HNAKTKPREEQ | |
| | GGACGTGAGCCACGAAGACC | | YNSTYRVVSVLT | |
| | CTGAGGTCAAGTTCAACTGGT | | VLHQDWLNGKE | |
| | ACGTGGACGGCGTGGAGGTG | | YKCAVSNKALPA | |
| | CATAATGCCAAGACAAAGCCG | | PIEKTISKAKGQP | |
| | CGGGAGGAGCAGTACAACAG | | REPQVYTLPPSR | |
| | CACGTACCGTGTGGTCAGCGT | | DELTKNQVSLTC | |
| | CCTCACCGTCCTGCACCAGGA | | LVKGFYPSDIAV | |
| | CTGGCTGAATGGCAAGGAATA | | EWESNGQPENN | |
| | CAAGTGCGCGGTCTCCAACAA | | YKTTPPVLDSDG | |
| | AGCCCTCCCAGCCCCCATCGA | | SFFLYSKLTVDK | |
| | GAAAACCATCTCCAAAGCCAA | | SRWQQGNVFSC | |
| | AGGGCAGCCCCGAGAACCAC | | SVMHEALHNHY | |
| | AGGTGTACACCCTGCCCCCAT | | TQKSLSLSPG | |
| | CCCGGGATGAGCTGACCAAG | | | |
| | AACCAGGTCAGCCTGACCTGC | | | |
| | CTGGTCAAAGGCTTCTATCCA | | | |
| | AGCGACATCGCCGTGGAGTG | | | |
| | GGAGAGCAATGGGCAGCCGG | | | |
| | AGAACAACTACAAGACCACGC | | | |
| | CTCCCGTGCTGGACTCCGACG | | | |
| | GCTCCTTCTTCCTCTACAGCA | | | |
| | AGCTCACCGTGGACAAGAGCA | | | |
| | GGTGGCAGCAGGGGAACGTC | | | |
| | TTCTCATGCTCCGTGATGCAT | | | |
| | GAGGCTCTGCACAACCACTAC | | | |
| | ACGCAGAAGAGCCTCTCCCTG | | | |
| | TCTCCGGGT | | | |

In certain embodiments, the immunoglobulin constant regions of each polypeptide chain of a homodimeric or heterodimeric protein described herein are identical to each other. In certain other embodiments, the immunoglobulin constant region of one polypeptide chain of a heterodimeric protein is different from the immunoglobulin constant region of the other polypeptide chain of the heterodimer. For example, one immunoglobulin constant region of a heterodimeric protein can contain a CH3 domain with a “knob” mutation, whereas the other immunoglobulin constant region of the heterodimeric protein can contain a CH3 domain with a “hole” mutation.

### Fc-Binding Domain Linker

In some embodiments, the polypeptide may further comprise a Fc-binding domain linker. In some embodiments, the Fc-binding domain linker can be used to link the immunoglobulin constant region to a C-terminal binding domain (e.g., a CD3 binding domain). In some embodiments, the Fc-binding domain linker can be used as a hinge domain and/or incorporated into an scFv. In some embodiments, the Fc-binding domain linker is a Gly₄Ser linker (SEQ ID NO: 128). In some embodiments, the Fc-binding domain linker is a 20mer consisting of four repeats of a Gly-Gly-Gly-Gly-Ser (SEQ ID NO: 128) amino acid sequence ((Gly₄Ser)₄) (SEQ ID NO:61). In some embodiments, the Fc-binding domain linker comprises an amino acid sequence selected from any one of SEQ ID NOs 50-70. Other linkers have been used, and phage display technology, as well as selective infective phage technology, has been used to diversify and select appropriate linker sequences (Tang et al., J. Biol. Chem. 271, 15682-15686, 1996; Hennecke et al., Protein Eng. 11, 405-410, 1998). In certain embodiments, the V_{L} and V_{H} regions are joined by a peptide linker having an amino acid sequence comprising the formula (Gly₄Ser)ₙ, wherein n=1-5 (SEQ ID NO: 129). Other suitable linkers can be obtained by optimizing a simple linker through random mutagenesis. In some embodiments, bispecific molecules do not comprise a hinge region or a constant region.

In certain embodiments, a Fc-binding domain linker is a flexible linker sequence comprising glycine-serine (e.g., Gly₄Ser, SEQ ID NO: 128) repeats. In certain embodiments, the linker comprises three Gly₄Ser repeats (SEQ ID NO: 59) followed by a proline residue. In certain embodiments the proline residue is followed by an amino acid selected from the group consisting of glycine, arginine and serine. In some embodiments, a Fc-binding domain linker comprises or consists of a sequence selected from SEQ ID NO: 50-70.

Some exemplary hinge, Fc-binding domain linker sequences suitable for use in accordance with the present disclosure are shown in Table 2. Additional exemplary hinge and linker regions are set forth in SEQ ID NOs: 241-244, 601, 78, 763-791, 228, 379-434, 618-749 of U.S. 2013/0129723 (said sequences incorporated by reference herein).

TABLE 2
Exemplary hinges and linkers

| Name | Amino Acid Sequence | SEQ ID NO |
|---|---|---|
| sss(s)-hlgG1 | EPKSSDKTHTSPPSS | SEQ ID NO: 36 |
| hinge | | |
| csc(s)-hlgG1 | EPKSCDKTHTSPPCS | SEQ ID NO: 37 |
| hinge | | |
| ssc(s)-hlgG1 | EPKSSDKTHTSPPCS | SEQ ID NO: 38 |
| hinge | | |
| scc(s)-hlgG1 | EPKSSDKTHTCPPCS | SEQ ID NO: 39 |
| hinge | | |
| css(s)-hlgG1 | EPKSCDKTHTSPPSS | SEQ ID NO: 40 |
| hinge | | |
| scs(s)-hlgG1 | EPKSSDKTHTCPPSS | SEQ ID NO: 41 |
| hinge | | |
| ccc(s)-hlgG1 | EPKSCDKTHTSPPCS | SEQ ID NO: 42 |
| hinge | | |
| ccc(p)-hlgG1 | EPKSCDKTHTSPPCP | SEQ ID NO: 43 |
| hinge | | |
| sss(p)-hlgG1 | EPKSSDKTHTSPPSP | SEQ ID NO: 44 |
| hinge | | |
| csc(p)-hlgG1 | EPKSCDKTHTSPPCP | SEQ ID NO: 45 |
| hinge | | |
| ssc(p)-hlgG1 | EPKSSDKTHTSPPCP | SEQ ID NO: 46 |
| hinge | | |
| scc(p)-hlgG1 | EPKSSDKTHTCPPCP | SEQ ID NO: 47 |
| hinge | | |
| css(p)-hlgG1 | EPKSCDKTHTSPPSP | SEQ ID NO: 48 |
| hinge | | |
| scs(p)-hlgG1 | EPKSSDKTHTCPPSP | SEQ ID NO: 49 |
| hinge | | |
| Scppcp | SCPPCP | SEQ ID NO: 50 |
| STD1 | NYGGGGSGGGGSGGG | SEQ ID NO: 51 |
| | GSGNS | |
| STD2 | NYGGGGSGGGGSGGG | SEQ ID NO: 52 |
| | GSGNYGGGGSGGGGS | |
| | GGGGSGNS | |
| H1 | NS | — |
| H2 | GGGGSGNS | SEQ ID NO: 53 |
| H3 | NYGGGGSGNS | SEQ ID NO: 54 |
| H4 | GGGGSGGGGSGNS | SEQ ID NO: 55 |
| H5 | NYGGGGSGGGGSGNS | SEQ ID NO: 56 |
| H6 | GGGGSGGGGSGGGGS | SEQ ID NO: 57 |
| | GNS | |
| H7 | GCPPCPNS | SEQ ID NO: 58 |
| (G₄S)₃ | GGGGSGGGGSGGGGS | SEQ ID NO: 59 |
| H105 | SGGGGSGGGGSGGGG | SEQ ID NO: 60 |
| | S | |
| (G₄S)₄ | GGGGSGGGGSGGGGS | SEQ ID NO: 61 |
| | GGGGS | |
| (G₄S)₅ | GGGGSGGGGSGGGGS | SEQ ID NO: 62 |
| | GGGGSGGGGS | |
| H94 | SGGGGSGGGGSGGGG | SEQ ID NO: 68 |
| | SPNS | |
| H111 | SGGGGSGGGGSGGGG | SEQ ID NO: 69 |
| | SPGS | |
| H114 | GGGGSGGGGSGGGGS | SEQ ID NO: 70 |
| | PS | |

In addition to the aforementioned domains, the therapeutic polypeptides can further comprise immunoglobulin dimerization/heterodimerization domains, junctional amino acids, tags, additional binding domains, etc. In some embodiments, the polypeptides and proteins described herein are conjugated to a drug or a toxic moiety.

### Bispecific/Multispecific Proteins

In some embodiments, a therapeutic protein may be a bispecific or multispecific protein. Non-limiting examples of bispecific molecules include an scFv-Fc-scFv molecule, an scFv-Ig molecule and an scFv-scFv molecule. In some embodiments, the bispecific molecules described herein comprise or consist of a first binding domain scFv linked to a second binding domain scFv and do not include other sequences such as an immunoglobulin constant region. In some embodiments, a therapeutic protein may be a bispecific or multispecific protein that comprises, from amino-terminus to carboxyl-terminus, or in order from carboxyl-terminus to amino-terminus, (i) a first binding domain, (ii) a hinge region, (iii) an immunoglobulin constant region, (iv) (optionally) a Fc-binding domain linker, and (v) a second binding domain.

In some embodiments, a multispecific protein may comprise, from N-terminus to C-terminus, a CD3 binding domain, a hinge region, an immunoglobulin constant region, and a tumor antigen binding domain. The tumor antigen binding domain may bind to, for example, CD123, PSMA, CD19, CD33, 5T4, or HER2.

In some embodiments, a multispecific protein may comprise, from N-terminus to C-terminus, a tumor antigen binding domain, a hinge region, an immunoglobulin constant region, and a CD3 binding domain. The tumor antigen binding domain may bind to, for example, CD123, PSMA, CD19, CD33, 5T4, or HER2.

In some embodiments, a multispecific protein may comprise, from N-terminus to C-terminus, the 4-1-BB binding domain, a hinge region, an immunoglobulin constant region, and a tumor antigen binding domain. The tumor antigen binding domain may bind to, for example, CD123, PSMA, CD19, CD33, 5T4, or HER2.

In some embodiments, a multispecific protein may comprise, from N-terminus to C-terminus, a tumor antigen binding domain, a hinge region, an immunoglobulin constant region, and a 4-1-BB binding domain. The tumor antigen binding domain may bind to, for example, CD123, PSMA, CD19, CD33, 5T4, or HER2.

### Homodimers/Heterodimers

In some embodiments, a therapeutic protein may be a homodimer or a heterodimer. In some embodiments, a therapeutic protein is a dimer of two identical polypeptides, wherein each polypeptide comprises, in order from amino-terminus to carboxyl-terminus, or in order from carboxyl-terminus to amino-terminus (i) a first binding domain, (ii) a hinge region, and (iii) an immunoglobulin constant region, (iv) (optionally) a Fc-binding domain linker, and (v) a second binding domain. In some embodiments, the bispecific or multispecific protein is a dimer of two identical polypeptides, wherein each polypeptide comprises, in order from amino-terminus to carboxyl-terminus, or in order from carboxyl-terminus to amino-terminus: (i) a first binding domain, (ii) a hinge region, (iii) an immunoglobulin constant region, (iv) (optionally) a Fc-binding domain linker, and (v) a second binding domain. In other embodiments, the bispecific proteins described herein are diabodies.

In certain embodiments, a hinge present in a polypeptide that forms a heterodimer with another polypeptide chain can be an immunoglobulin hinge, such as a wild-type immunoglobulin hinge region or an altered immunoglobulin hinge region thereof. In certain embodiments, a hinge of one polypeptide chain of a heterodimeric protein is identical to a corresponding hinge of the other polypeptide chain of the heterodimer. In certain other embodiments, a hinge of one chain is different from that of the other chain (in their length or sequence). The different hinges in the different chains allow different manipulation of the binding affinities of the binding domains to which the hinges are connected, so that the heterodimer is able to preferentially bind to the target of one binding domain over the target of the other binding domain.

In other embodiments, the polypeptides and proteins described herein include a heterodimerization domain that is capable of heterodimerization with a different heterodimerization domain in a second, non-identical polypeptide chain. In certain variations, the second polypeptide chain for heterodimerization includes a second binding domain. Accordingly, in certain embodiments of the present disclosure, two non-identical polypeptide chains, one comprising the polypeptide comprising a first binding domain and the second optionally comprising a second binding domain, dimerize to form a heterodimeric binding protein. Dimerization/heterodimerization domains can be used where it is desired to form heterodimers from two non-identical polypeptide chains, where one or both polypeptide chains comprise a binding domain. In certain embodiments, one polypeptide chain member of certain heterodimers described herein does not contain a binding domain. Examples of types of heterodimers include those described in U.S. Patent Application Publication Nos. 2013/0095097 and 2013/0129723, and International PCT Publication No. WO 2016/094873.

In certain embodiments, the first and second polypeptide chains dimerize via the inclusion of an “immunoglobulin dimerization domain” or “immunoglobulin heterodimerization domain.” An “immunoglobulin dimerization domain” or “immunoglobulin heterodimerization domain” refers herein to an immunoglobulin domain of a first polypeptide chain that preferentially interacts or associates with a different immunoglobulin domain of a second polypeptide chain, wherein the interaction of the different immunoglobulin domains substantially contributes to or efficiently promotes heterodimerization of the first and second polypeptide chains (i.e., the formation of a dimer between two different polypeptide chains, which is also referred to as a “heterodimer”). The immunoglobulin heterodimerization domains in the polypeptide chains of a heterodimer are different from each other and thus can be differentially modified to facilitate heterodimerization of both chains and to minimize homodimerization of either chain. Immunoglobulin heterodimerization domains provided herein allow for efficient heterodimerization between different polypeptides and facilitate purification of the resulting heterodimeric protein.

As provided herein, immunoglobulin heterodimerization domains useful for promoting heterodimerization of two different polypeptide chains according to the present disclosure include wild-type and altered immunoglobulin CH1 and CL domains, for instance, human CH1 and CL domains. In certain embodiments, an immunoglobulin heterodimerization domain is a wild-type CH1 domain, such as a wild type IgG1, IgG2, IgG3, IgG4, IgA1, IgA2, IgD, IgE, or IgM CH1 domain, for example, as set forth in SEQ ID NOs: 114, 186-192 and 194, respectively, of U.S. Patent Application Publication No. 2013/0129723 or SEQ ID NO: 114 of U.S. Patent Application Publication No. 2013/0129723 (said sequence incorporated by reference herein). In further embodiments, a cysteine residue of a wild-type CH1 domain (e.g., a human CH1) involved in forming a disulfide bond with a wild type immunoglobulin CL domain (e.g., a human CL) is deleted or substituted in the altered immunoglobulin CH1 domain such that a disulfide bond is not formed between the altered CH1 domain and the wild-type CL domain.

Polypeptides and proteins described herein may be made using scaffolding as generally disclosed in U.S. Patent Application Publication Nos. 2013/0129723 and 2013/0095097, which are each incorporated herein by reference in their entirety. The polypeptides described herein may comprise two non-identical polypeptide chains, each polypeptide chain comprising an immunoglobulin heterodimerization domain. The interfacing immunoglobulin heterodimerization domains are different. In one embodiment, the immunoglobulin heterodimerization domain comprises a CH1 domain or a derivative thereof. In another embodiment, the immunoglobulin heterodimerization domain comprises a CL domain or a derivative thereof. In one embodiment, the CL domain is a Cκ or Cλ isotype or a derivative thereof.

### Exemplary Protein Therapeutics: Anti-CD123×Anti-CD3 Polypeptides and Dimers Thereof

An exemplary protein therapeutic may bind both CD123-expressing cells and the T-cell receptor complex on T-cells to induce target-dependent T-cell cytotoxicity, activation and proliferation.

Thus, in certain embodiments, the therapeutic protein used in connection with the methods and compositions described herein is a bispecific single chain molecule comprising a CD123 binding domain and a CD3 binding domain. In some embodiments, a CD123 and/or a CD3 binding domain is derived from an antibody and comprises a variable heavy chain (VH) and a variable light chain (VL). For example, the CD123 and/or CD3 binding domains may be an scFv that comprises a VH and VL. These binding domains and variable chains may be arranged in any order that still retains some binding to the target(s). For example, the variable domains may be arranged in the order such as (VH CD123)-(VL CD123)-(VH CD3)-(VL CD3); (VL CD123)-(VH CD123)-(VH CD3)-(VL CD3); (VH CD123)-(VL CD123)-(VL CD3)-(VH CD3); (VL CD123)-(VH CD123)-(VL CD3)-(VH CD3); (VH CD3)-(VL CD3)-(VH CD123)-(VL CD123); (VL CD3)-(VH CD3)-(VL CD123)-(VH CD123); (VH CD3)-(VL CD3)-(VL CD123)-(VH CD123); or (VL CD3)-(VH CD3)-(VH CD123)-(VL CD123). The pairs of VH regions and VL regions in the binding domain binding to CD3 may be in the format of a single chain antibody (scFv). The VH and VL regions may be arranged in the order VH-VL or VL-VH. In some embodiments, the scFv may bind to CD123 more effectively than the antibody comprising the same VH and VL region sequences in the same orientation. In certain embodiments, the scFv may bind more effectively to CD123 in the VL-VH orientation than in the VH-VL orientation, or vice versa. The VH-region may be positioned N-terminally to a linker sequence. The VL region may be positioned C-terminally to the linker sequence. The domain arrangement in the CD3 binding domain of the bispecific single chain molecule may be VH-VL, with the CD3 binding domain located C-terminally to the CD123-binding domain. A bispecific molecule may comprise an scFv binding to CD123 linked to an scFv binding to CD3. These scFvs may be linked with a short peptide. In some embodiments, bispecific single chain molecules do not comprise a hinge region or a constant region (see, for example, US 2013/0295121, WO 2010/037836, WO 2004/106381 and WO 2011/121110; each incorporated herein by reference in its entirety).

The CD123-bispecific binding construct may comprise one or more sequences shown in Table 3, Table 4, and/or Table 5.

TABLE 3
Binding Polypeptide Sequences and Components

| | | | SEQ ID |
|---|---|---|---|
| | | | NOS: |
| | | Amino Acid | nucleotide |
| Name | Nucleotide Sequence | Sequence | (amino acid) |
| OMT1 | gacatcgtgatgacccagtctccagactccctg | divmtqspdsl | SEQ ID |
| variable | gctgtgtctctgggcgagagggccaccatcaac | avslgeratin | NO: 133 |
| light | tgcaagtccagccacagtgttttatacagctcc | cksshsvlyss | (SEQ ID |
| chain | aacaataagaactacttagcttggtaccagcag | nnknylawyqq | NO: 134) |
| domain | aaaccaggacagcctcctaagctgctcatttac | kpgqppklliy | |
| | tgggcatctacccgggaatccggggtccctgac | wastresgvpd | |
| | cgattcagtggcagcgggtctgggacagatttc | rfsgsgsgtdf | |
| | actctcaccatcagcagcctgcaggctgaagat | tltisslqaed | |
| | gtggcagtttattactgtcagcaatattatagt | vavyycqqyys | |
| | actcctccgaccactttcggcggagggaccaag | tppttfgggtk | |
| | gtggagatcaaa | veik | |
| OMT1 | gaggtgcagctgttggagtctgggggaggcttg | evqllesgggl | SEQ ID |
| variable | gtacagcctggggggtccctgagactctcctgt | vqpggslrlsc | NO: 135 |
| heavy | gcagcctctggattcacctttagcagctatggc | aasgftfssyg | (SEQ ID |
| chain | atgagctgggtccgccaggctccagggaagggg | mswvrqapgkg | NO: 136) |
| domain | ctggagggggtctcagctattagtggtagtggt | legvsaisgsg | |
| | ggtagcacatactacgcagactccgtgaagggc | gstyyadsvkg | |
| | cggttcaccatctccagagacaattccaagaac | rftisrdnskn | |
| | acgctgtatctgcaaatgaacagcctgagagcc | tlylqmnslra | |
| | gaggacacggccgtatattactgtgcgaaagaa | edtavyycake | |
| | aagttacgatattttgactggttatccgatgct | klryfdwlsda | |
| | tttgatatctggggccaagggacaatggtcacc | fdiwgqgtmvt | |
| | gtctcttca | vss | |
| OMT1 | cacagtgttttatacagctccaacaataagaac | HSVLYSSNNKN | SEQ ID |
| CDR L1 | tac | Y | NO: 137 |
| | | | (SEQ ID |
| | | | NO: 138) |
| OMT1 | tgggcatct | WAS | SEQ ID |
| CDR L2 | | | NO: 139 |
| | | | (SEQ ID |
| | | | NO: 140) |
| OMT1 | cagcaatattatagtactcctccgaccact | QQYYSTPPTT | SEQ ID |
| CDR L3 | | | NO: 141 |
| | | | (SEQ ID |
| | | | NO: 142) |
| OMT1 | ggattcacctttagcagctatggc | GFTFSSYG | SEQ ID |
| CDR H1 | | | NO: 143 |
| | | | (SEQ ID |
| | | | NO: 144) |
| OMT1 | attagtggtagtggtggtagcaca | ISGSGGST | SEQ ID |
| CDR H2 | | | NO: 145 |
| | | | (SEQ ID |
| | | | NO: 146) |
| OMT1 | gcgaaagaaaagttacgatattttgactggtta | AKEKLRYFDWL | SEQ ID |
| CDR H3 | tccgatgcttttgatatc | SDAFDI | NO: 147 |
| | | | (SEQ ID |
| | | | NO: 148) |
| MT1 | atggaagcaccagcgcagcttctcttcctcctg | evqllesgggl | SEQ ID |
| VHVL x | ctactctggctcccagataccaccggtgaggtg | vqpggslrlsc | NO: 309 |
| TSC456 | cagctgttggagtctgggggaggcttggtacag | aasgftfssyg | (SEQ ID |
| scFv-Fc- | cctggggggtccctgagactctcctgtgcagcc | mswvrqapgkg | NO: 310) |
| scFv | tctggattcacctttagcagctatggcatgagc | legvsaisgsg | |
| TRI129 | tgggtccgccaggctccagggaaggggctggag | gstyyadsvkg | |
| | ggggtctcagctattagtggtagtggtggtagc | rftisrdnskn | |
| | acatactacgcagactccgtgaagggccggttc | tlylqmnslra | |
| | accatctccagagacaattccaagaacacgctg | edtavyycake | |
| | tatctgcaaatgaacagcctgagagccgaggac | klryfdwlsda | |
| | acggccgtatattactgtgcgaaagaaaagtta | fdiwgqgtmvt | |
| | cgatattttgactggttatccgatgcttttgat | vssggggsggg | |
| | atctggggccaagggacaatggtcaccgtctct | gsggggsgggg | |
| | tcaggtggaggcggttcaggcggaggtggatcc | sdivmtqspds | |
| | ggcggtggcggctccggtggcggcggatctgac | lavslgerati | |
| | atcgtgatgacccagtctccagactccctggct | ncksshsvlys | |
| | gtgtctctgggcgagagggccaccatcaactgc | snnknylawyq | |
| | aagtccagccacagtgttttatacagctccaac | qkpgqppklli | |
| | aataagaactacttagcttggtaccagcagaaa | ywastresgvp | |
| | ccaggacagcctcctaagctgctcatttactgg | drfsgsgsgtd | |
| | gcatctacccgggaatccggggtccctgaccga | ftltisslqae | |
| | ttcagtggcagcgggtctgggacagatttcact | dvavyycqqyy | |
| | ctcaccatcagcagcctgcaggctgaagatgtg | stppttfgggt | |
| | gcagtttattactgtcagcaatattatagtact | kveiksssepk | |
| | cctccgaccactttcggcggagggaccaaggtg | ssdkthtcppc | |
| | gagatcaaatcctcgagtgagcccaaatcttct | papeaagapsv | |
| | gacaaaactcacacatgcccaccgtgcccagca | flfppkpkdtl | |
| | cctgaagccgcgggtgcaccgtcagtcttcctc | misrtpevtcv | |
| | ttccccccaaaacccaaggacaccctcatgatc | vvdvshedpev | |
| | tcccggacccctgaggtcacatgcgtggtggtg | kfnwyvdgvev | |
| | gacgtgagccacgaagaccctgaggtcaagttc | hnaktkpreeq | |
| | aactggtacgtggacggcgtggaggtgcataat | ynstyrvvsvl | |
| | gccaagacaaagccgcgggaggagcagtacaac | tvlhqdwlngk | |
| | agcacgtaccgtgtggtcagcgtcctcaccgtc | eykcavsnkal | |
| | ctgcaccaggactggctgaatggcaaggaatac | papiektiska | |
| | aagtgcgcggtctccaacaaagccctcccagcc | kgqprepqvyt | |
| | cccatcgagaaaaccatctccaaagccaaaggg | lppsrdeltkn | |
| | cagccccgagaaccacaggtgtacaccctgccc | qvsltclvkgf | |
| | ccatcccgggatgagctgaccaagaaccaggtc | ypsdiavewes | |
| | agcctgacctgcctggtcaaaggcttctatcca | ngqpennyktt | |
| | agcgacatcgccgtggagtgggagagcaatggg | ppvldsdgsff | |
| | cagccggagaacaactacaagaccacgcctccc | lyskltvdksr | |
| | gtgctggactccgacggctccttcttcctctac | wqqgnvfscsv | |
| | agcaagctcaccgtggacaagagcaggtggcag | mhealhnhytq | |
| | caggggaacgtcttctcatgctccgtgatgcat | kslslspgsgg | |
| | gaggctctgcacaaccactacacgcagaagagc | ggsggggsggg | |
| | ctctccctgtctccgggttccggaggagggggt | gspsqvqlvqs | |
| | tcaggtgggggaggttctggcggcgggggaagc | gpevkkpgssv | |
| | ccttcacaggtgcaactggtgcagagtggaccc | kvsckasgytf | |
| | gaggttaaaaaaccagggtcctccgttaaggtt | srstmhwvrqa | |
| | agctgcaaagcctctggctacacattttccagg | pgqglewigyi | |
| | agtacaatgcactgggtgaggcaggctcctgga | npssaytnynq | |
| | cagggactcgagtggatcgggtatatcaaccca | kfkdrvtitad | |
| | tctagcgcctataccaattacaaccaaaagttt | kststaymels | |
| | aaggaccgagttaccattaccgctgacaaatcc | slrsedtavyy | |
| | accagtacagcttatatggagctgtcatctctt | carpqvhydyn | |
| | aggtccgaggacactgctgtttattactgcgct | gfpywgqgtlv | |
| | cgtcctcaggttcactatgactataatggtttt | tvssggggsgg | |
| | ccctactggggtcagggaaccctggtgactgtc | ggsggggsggg | |
| | tcttctggcggtggaggcagcggtgggggtggg | gsdiqmtqsps | |
| | tctggaggcggtggcagtggcggcggaggctct | tlsasvgdrvt | |
| | gatattcagatgactcagtctcctagcactctc | mtcsasssvsy | |
| | agcgccagcgtgggggatcgtgtgacaatgact | mnwyqqkpgka | |
| | tgctccgctagcagtagtgtgtcttacatgaat | pkrwiydsskl | |
| | tggtatcagcagaagcccgggaaagcacctaag | asgvpsrfsgs | |
| | cgctggatctatgactcttccaagctggcaagt | gsgtdytltis | |
| | ggtgtcccctcacggttctctggctcaggttct | slqpddfatyy | |
| | ggtactgactatactttgactatctcctccctc | cqqwsrnpptf | |
| | cagcccgatgatttcgctacctattattgtcag | gggtkveikrs | |
| | cagtggagccgtaacccacccactttcggaggc | | |
| | ggtaccaaagtggagatcaagaggtcataa | | |
| OMT1 | atggaagcaccagcgcagcttctcttcctcctg | divmtqspdsl | SEQ ID |
| VLVH x | ctactctggctcccagataccaccggtgacatc | avslgeratin | NO: 311 |
| TSC456 | gtgatgacccagtctccagactccctggctgtg | cksshsvlyss | (SEQ ID |
| scFv-Fc- | tctctgggcgagagggccaccatcaactgcaag | nnknylawyqq | NO: 312) |
| scFv | tccagccacagtgttttatacagctccaacaat | kpgqppklliy | |
| TRI130 | aagaactacttagcttggtaccagcagaaacca | wastresgvpd | |
| | ggacagcctcctaagctgctcatttactgggca | rfsgsgsgtdf | |
| | tctacccgggaatccggggtccctgaccgattc | tltisslqaed | |
| | agtggcagcgggtctgggacagatttcactctc | vavyycqqyys | |
| | accatcagcagcctgcaggctgaagatgtggca | tppttfgggtk | |
| | gtttattactgtcagcaatattatagtactcct | veikggggsgg | |
| | ccgaccactttcggcggagggaccaaggtggag | ggsggggsggg | |
| | atcaaaggtggaggcggttcaggcggaggtgga | gsevqllesgg | |
| | tccggcggtggcggctccggtggcggcggatct | glvqpggslrl | |
| | gaggtgcagctgttggagtctgggggaggcttg | scaasgftfss | |
| | gtacagcctggggggtccctgagactctcctgt | ygmswvrqapg | |
| | gcagcctctggattcacctttagcagctatggc | kglegvsaisg | |
| | atgagctgggtccgccaggctccagggaagggg | sggstyyadsv | |
| | ctggagggggtctcagctattagtggtagtggt | kgrftisrdns | |
| | ggtagcacatactacgcagactccgtgaagggc | kntlylqmnsl | |
| | cggttcaccatctccagagacaattccaagaac | raedtavyyca | |
| | acgctgtatctgcaaatgaacagcctgagagcc | keklryfdwls | |
| | gaggacacggccgtatattactgtgcgaaagaa | dafdiwgqgtm | |
| | aagttacgatattttgactggttatccgatgct | vtvsssepkss | |
| | tttgatatctggggccaagggacaatggtcacc | dkthtcppcpa | |
| | gtctcctcgagtgagcccaaatcttctgacaaa | peaagapsvfl | |
| | actcacacatgcccaccgtgcccagcacctgaa | fppkpkdtlmi | |
| | gccgcgggtgcaccgtcagtcttcctcttcccc | srtpevtcvvv | |
| | ccaaaacccaaggacaccctcatgatctcccgg | dvshedpevkf | |
| | acccctgaggtcacatgcgtggtggtggacgtg | nwyvdgvevhn | |
| | agccacgaagaccctgaggtcaagttcaactgg | aktkpreeqyn | |
| | tacgtggacggcgtggaggtgcataatgccaag | styrvvsvltv | |
| | acaaagccgcgggaggagcagtacaacagcacg | lhqdwlngkey | |
| | taccgtgtggtcagcgtcctcaccgtcctgcac | kcavsnkalpa | |
| | caggactggctgaatggcaaggaatacaagtgc | piektiskakg | |
| | gcggtctccaacaaagccctcccagcccccatc | qprepqvytlp | |
| | gagaaaaccatctccaaagccaaagggcagccc | psrdeltknqv | |
| | cgagaaccacaggtgtacaccctgcccccatcc | sltclvkgfyp | |
| | cgggatgagctgaccaagaaccaggtcagcctg | sdiavewesng | |
| | acctgcctggtcaaaggcttctatccaagcgac | qpennykttpp | |
| | atcgccgtggagtgggagagcaatgggcagccg | vldsdgsffly | |
| | gagaacaactacaagaccacgcctcccgtgctg | skltvdksrwq | |
| | gactccgacggctccttcttcctctacagcaag | qgnvfscsvmh | |
| | ctcaccgtggacaagagcaggtggcagcagggg | ealhnhytqks | |
| | aacgtcttctcatgctccgtgatgcatgaggct | lslspgsgggg | |
| | ctgcacaaccactacacgcagaagagcctctcc | sggggsggggs | |
| | ctgtctccgggttccggaggagggggttcaggt | psqvqlvqsgp | |
| | gggggaggttctggcggcgggggaagcccttca | evkkpgssvkv | |
| | caggtgcaactggtgcagagtggacccgaggtt | sckasgytfsr | |
| | aaaaaaccagggtcctccgttaaggttagctgc | stmhwvrqapg | |
| | aaagcctctggctacacattttccaggagtaca | qglewigyinp | |
| | atgcactgggtgaggcaggctcctggacaggga | ssaytnynqkf | |
| | ctcgagtggatcgggtatatcaacccatctagc | kdrvtitadks | |
| | gcctataccaattacaaccaaaagtttaaggac | tstaymelssl | |
| | cgagttaccattaccgctgacaaatccaccagt | rsedtavyyca | |
| | acagcttatatggagctgtcatctcttaggtcc | rpqvhydyngf | |
| | gaggacactgctgtttattactgcgctcgtcct | pywgqgtlvtv | |
| | caggttcactatgactataatggttttccctac | ssggggsgggg | |
| | tggggtcagggaaccctggtgactgtctcttct | sggggsggggs | |
| | ggcggtggaggcagcggtgggggtgggtctgga | diqmtqspstl | |
| | ggcggtggcagtggcggcggaggctctgatatt | sasvgdrvtmt | |
| | cagatgactcagtctcctagcactctcagcgcc | csasssvsymn | |
| | agcgtgggggatcgtgtgacaatgacttgctcc | wyqqkpgkapk | |
| | gctagcagtagtgtgtcttacatgaattggtat | rwiydssklas | |
| | cagcagaagcccgggaaagcacctaagcgctgg | gvpsrfsgsgs | |
| | atctatgactcttccaagctggcaagtggtgtc | gtdytltissl | |
| | ccctcacggttctctggctcaggttctggtact | qpddfatyycq | |
| | gactatactttgactatctcctccctccagccc | qwsrnpptfgg | |
| | gatgatttcgctacctattattgtcagcagtgg | gtkveikrs | |
| | agccgtaacccacccactttcggaggcggtacc | | |
| | aaagtggagatcaagaggtcataa | | |
| Cris7 | | RSTMH | (SEQ ID |
| and | | | NO: 345) |
| DRA222 | | | |
| VH | | | |
| CDR1 | | | |
| (Kabat) | | | |
| Cris7 | | YINPSSAYTNY | (SEQ ID |
| and | | NQKFK | NO: 346) |
| DRA222 | | | |
| VH | | | |
| CDR2 | | | |
| (Kabat) | | | |
| Cris7 | | QVHYDYNGFPY | (SEQ ID |
| and | | | NO: 347) |
| DRA222 | | | |
| VH | | | |
| CDR3 | | | |
| (Kabat) | | | |
| Cris7 | | SASSSVSYMN | (SEQ ID |
| and | | | NO: 348) |
| DRA222 | | | |
| VL | | | |
| CDR1 | | | |
| (Kabat) | | | |
| Cris7 | | DSSKLAS | (SEQ ID |
| and | | | NO: 349) |
| DRA222 | | | |
| VL | | | |
| CDR2 | | | |
| (Kabat) | | | |
| Cris7 | | QQWSRNPPT | (SEQ ID |
| and | | | NO: 350) |
| DRA222 | | | |
| VL | | | |
| CDR3 | | | |
| (Kabat) | | | |
| Cris7 | | GYTFTRST | (SEQ ID |
| and | | | NO: 351) |
| DRA222 | | | |
| VH | | | |
| CDR1 | | | |
| (IMGT) | | | |
| Cris7 | | INPSSAYT | (SEQ ID |
| and | | | NO: 352) |
| DRA222 | | | |
| VH | | | |
| CDR2 | | | |
| (IMGT) | | | |
| Cris7 | | QQWSRNPPT | (SEQ ID |
| and | | | NO: 353) |
| DRA222 | | | |
| VH | | | |
| CDR3 | | | |
| (IMGT) | | | |
| Cris7 | | ASSSVSY | (SEQ ID |
| and | | | NO: 354) |
| DRA222 | | | |
| VL | | | |
| CDR1 | | | |
| (IMGT) | | | |
| Cris7 | | DSS | (SEQ ID |
| and | | | NO: 355) |
| DRA222 | | | |
| VL | | | |
| CDR2 | | | |
| (IMGT) | | | |
| Cris7 | | QQWSRNPPT | (SEQ ID |
| and | | | NO: 356) |
| DRA222 | | | |
| VL | | | |
| CDR3 | | | |
| (IMGT) | | | |
| Cris-7 | | QVVLTQSPAIM | (SEQ ID |
| variable | | SAFPGEKVTMT | NO: 341) |
| light | | CSASSSVSYMN | |
| chain | | WYQQKSGTSPK | |
| sequence | | RWIYDSSKLAS | |
| | | GVPARFSGSGS | |
| | | GTSYSLTISSM | |
| | | ETEDAATYYCO | |
| | | QWSRNPPTEGG | |
| | | GTKLQITR | |
| Cris-7 | | QVQLQQSGAEL | (SEQ ID |
| variable | | ARPGASVKMSC | NO: 342) |
| heavy | | KASGYTFTRST | |
| chain | | MHWVKQRPGQG | |
| sequence | | LEWIGYINPSS | |
| | | AYTNYNQKFKD | |
| | | KATLTADKSSS | |
| | | TAYMQLSSLTS | |
| | | EDSAVYYCASP | |
| | | QVHYDYNGFPY | |
| | | WGQGTLVTVSA | |
| HuM291 | | DIQMTQSPSSL | (SEQ ID |
| variable | | SASVGDRVTIT | NO: 343) |
| light | | CSASSSVSYMN | |
| chain | | WYQQKPGKAPK | |
| sequence | | RLIYDTSKLAS | |
| | | GVPSRFSGSGS | |
| | | GTDETLTISSL | |
| | | QPEDFATYYCQ | |
| | | QWSSNPPTFGG | |
| | | GTKVEIK | |
| HuM291 | | QVQLVQSGAEV | (SEQ ID |
| variable | | KKPGASVKVSC | NO: 344) |
| heavy | | KASGYTFISYT | |
| chain | | MHWVRQAPGQG | |
| sequence | | LEWMGYINPRS | |
| | | GYTHYNQKLKD | |
| | | KATLTADKSAS | |
| | | TAYMELSSLRS | |
| | | EDTAVYYCARS | |
| | | AYYDYDGFAYW | |
| | | GQGTLVTVSS | |
| 12C VH | | KYAMN | (SEQ ID |
| CDR1 | | | NO: 357) |
| (Kabat) | | | |
| 12C VH | | RIRSKYNNYAT | (SEQ ID |
| CDR2 | | YYADSVKD | NO: 358) |
| (Kabat) | | | |
| 12C VH | | HGNFGNSYISY | (SEQ ID |
| CDR3 | | WAY | NO: 359) |
| (Kabat) | | | |
| 12C VL | | GSSTGAVTSGN | (SEQ ID |
| CDR1 | | YPN | NO: 360) |
| (Kabat) | | | |
| 12C VL | | GTKFLAP | (SEQ ID |
| CDR2 | | | NO: 361) |
| (Kabat) | | | |
| 12C VL | | VLWYSNRWV | (SEQ ID |
| CDR3 | | | NO: 362) |
| (Kabat) | | | |
| 12C VH | | GFTFNKYA | (SEQ ID |
| CDR1 | | | NO: 363) |
| (IMGT) | | | |
| 12C VH | | IRSKYNNYAT | (SEQ ID |
| CDR2 | | | NO: 364) |
| (IMGT) | | | |
| 12C VH | | VRHGNFGNSYI | (SEQ ID |
| CDR3 | | SYWAY | NO: 365) |
| (IMGT) | | | |
| 12C VL | | TGAVTSGNY | (SEQ ID |
| CDR1 | | | NO: 366) |
| (IMGT) | | | |
| 12C VL | | GTK | (SEQ ID |
| CDR2 | | | NO: 367) |
| (IMGT) | | | |
| 12C VL | | VLWYSNRWV | (SEQ ID |
| CDR3 | | | NO: 368) |
| (IMGT) | | | |
| HuM291 | | SYTMH | (SEQ ID |
| VH | | | NO: 369) |
| CDR1 | | | |
| (Kabat) | | | |
| HuM291 | | YINPRSGYTHY | (SEQ ID |
| VH | | NQKLKD | NO: 370) |
| CDR2 | | | |
| (Kabat) | | | |
| HuM291 | | SAYYDYDGFAY | (SEQ ID |
| VH | | | NO: 371) |
| CDR3 | | | |
| (Kabat) | | | |
| HuM291 | | SASSSVSYMN | (SEQ ID |
| VL | | | NO: 372) |
| CDR1 | | | |
| (Kabat) | | | |
| HuM291 | | DTSKLAS | (SEQ ID |
| VL | | | NO: 373) |
| CDR2 | | | |
| (Kabat) | | | |
| HuM291 | | QQWSSNPPT | (SEQ ID |
| VL | | | NO: 374) |
| CDR3 | | | |
| (Kabat) | | | |
| HuM291 | | GYTFISYT | (SEQ ID |
| VH | | | NO: 375) |
| CDR1 | | | |
| (IMGT) | | | |
| HuM291 | | INPRSGYT | (SEQ ID |
| VH | | | NO: 376) |
| CDR2 | | | |
| (IMGT) | | | |
| HuM291 | | ARSAYYDYDGF | (SEQ ID |
| VH | | AY | NO: 377) |
| CDR3 | | | |
| (IMGT) | | | |
| HuM291 | | ASSSVSY | (SEQ ID |
| VL | | | NO: 378) |
| CDR1 | | | |
| (IMGT) | | | |
| HuM291 | | DTS | (SEQ ID |
| VL | | | NO: 379) |
| CDR2 | | | |
| (IMGT) | | | |
| HuM291 | | QQWSSNPPT | (SEQ ID |
| VL | | | NO: 380) |
| CDR3 | | | |
| (IMGT) | | | |
| TSC455 | | QVQLVQSGPEV | (SEQ ID |
| (anti- | | KKPGSSVKVSC | NO: 381) |
| CD3) | | KASGYTFSRST | |
| TSC394 | | MHWVRQAPGQG | |
| F87Y | | LEWIGYINPSS | |
| scFv | | AYTNYNQKFKD | |
| | | RVTITADKSTS | |
| | | TAYMELSSLRS | |
| | | EDTAVYYCARP | |
| | | QVHYDYNGFPY | |
| | | WGQGTLVTVSS | |
| | | GGGGSGGGGSG | |
| | | GGGSGGGGSDI | |
| | | QMTQSPSTLSA | |
| | | SVGDRVTMTCS | |
| | | ASSSVSYMNWY | |
| | | QQKPGKAPKRW | |
| | | IYDSSKLASGV | |
| | | PSRFSGSGSGT | |
| | | EYTLTISSLQP | |
| | | DDFATYYCQQW | |
| | | SRNPPTFGGGT | |
| | | KVEIKRSSS | |
| TSC456 | | QVQLVQSGPEV | (SEQ ID |
| (anti- | | KKPGSSVKVSC | NO: 382) |
| CD3) | | KASGYTFSRST | |
| TSC394 | | MHWVRQAPGQG | |
| E86D | | LEWIGYINPSS | |
| F87Y | | AYTNYNQKFKD | |
| scFv | | RVTITADKSTS | |
| | | TAYMELSSLRS | |
| | | EDTAVYYCARP | |
| | | QVHYDYNGFPY | |
| | | WGQGTLVTVSS | |
| | | GGGGSGGGGSG | |
| | | GGGSGGGGSDI | |
| | | QMTQSPSTLSA | |
| | | SVGDRVTMTCS | |
| | | ASSSVSYMNWY | |
| | | QQKPGKAPKRW | |
| | | IYDSSKLASGV | |
| | | PSRFSGSGSGT | |
| | | DYTLTISSLQP | |
| | | DDFATYYCQQW | |
| | | SRNPPTFGGGT | |
| | | KVEIKRSSS | |
| TSC455 | | QVQLVQSGPEV | (SEQ ID |
| and | | KKPGSSVKVSC | NO: 383) |
| TSC456 | | KASGYTFSRST | |
| variable | | MHWVRQAPGQG | |
| heavy | | LEWIGYINPSS | |
| domain | | AYTNYNQKFKD | |
| | | RVTITADKSTS | |
| | | TAYMELSSLRS | |
| | | EDTAVYYCARP | |
| | | QVHYDYNGFPY | |
| | | WGQGTLVTVSS | |
| TSC455 | | DIQMTQSPSTL | (SEQ ID |
| variable | | SASVGDRVTMT | NO: 384) |
| light | | CSASSSVSYMN | |
| domain | | WYQQKPGKAPK | |
| | | RWIYDSSKLAS | |
| | | GVPSRFSGSGS | |
| | | GTEYTLTISSL | |
| | | QPDDFATYYCQ | |
| | | QWSRNPPTFGG | |
| | | GTKVEIKRS | |
| TSC456 | | DIQMTQSPSTL | (SEQ ID |
| variable | | SASVGDRVTMT | NO: 385) |
| light | | CSASSSVSYMN | |
| domain | | WYQQKPGKAPK | |
| | | RWIYDSSKLAS | |
| | | GVPSRFSGSGS | |
| | | GTDYTLTISSL | |
| | | QPDDFATYYCQ | |
| | | QWSRNPPTFGG | |
| | | GTKVEIKRS | |
| DRA222 | | QVQLVESGGGV | (SEQ ID |
| (anti- | | VQPGRSLRLSC | NO: 386) |
| CD3) | | KASGYTFTRST | |
| scFv | | MHWVRQAPGQG | |
| | | LEWIGYINPSS | |
| | | AYTNYNQKFKD | |
| | | RFTISADKSKS | |
| | | TAFLOMDSLRP | |
| | | EDTGVYFCARP | |
| | | QVHYDYNGFPY | |
| | | WGQGTPVTVSS | |
| | | GGGGSGGGGSG | |
| | | GGGSAQDIQMT | |
| | | QSPSSLSASVG | |
| | | DRVTMTCSASS | |
| | | SVSYMNWYQQK | |
| | | PGKAPKRWIYD | |
| | | SSKLASGVPAR | |
| | | FSGSGSGTDYT | |
| | | LTISSLQPEDF | |
| | | ATYYCQQWSRN | |
| | | PPTFGGGTKLQ | |
| | | ITSSS | |
| DRA222 | | QVQLVESGGGV | (SEQ ID |
| variable | | VQPGRSLRLSC | NO: 387) |
| heavy | | KASGYTFTRST | |
| domain | | MHWVRQAPGQG | |
| | | LEWIGYINPSS | |
| | | AYTNYNQKFKD | |
| | | RFTISADKSKS | |
| | | TAFLQMDSLRP | |
| | | EDTGVYFCARP | |
| | | QVHYDYNGFPY | |
| | | WGQGTPVTVSS | |
| DRA222 | | DIQMTQSPSSL | (SEQ ID |
| variable | | SASVGDRVTMT | NO: 388) |
| light | | CSASSSVSYMN | |
| domain | | WYQQKPGKAPK | |
| | | RWIYDSSKLAS | |
| | | GVPARFSGSGS | |
| | | GTDYTLTISSL | |
| | | QPEDFATYYCQ | |
| | | QWSRNPPTFGG | |
| | | GTKLQITS | |

TABLE 4
Composition of Humanized Constructs

| | scFv | Nucleotide | Amino acid |
|---|---|---|---|
| Construct ID | Orientation | SEQ ID NO | SEQ ID NO |
| TRI129 | VHVL | 309 | 310 |
| TRI130 | VLVH | 311 | 312 |

TABLE 5
Amino acid sequences of exemplary binding protein construct

| Construct | | SEQ |
|---|---|---|
| name | Sequence | ID NO |
| TRI130 | MEAPAQLLFLLLLWLPDTTGDIVMTQSPDSLAVSLGERATINCKSSHSV | 337 |
| (CD123 | LYSSNNKNYLAWYQQKPGQPPKLLIYWASTRESGVPDRFSGSGSGTDFT | |
| binding | LTISSLQAEDVAVYYCQQYYSTPPTTFGGGTKVEIKGGGGSGGGGSGGG | |
| domain in | GSGGGGSEVQLLESGGGLVQPGGSLRLSCAASGFTFSSYGMSWVRQAPG | |
| bold, CD3 | KGLEGVSAISGSGGSTYYADSVKGRFTISRDNSKNTLYLQMNSLRAEDT | |
| binding | AVYYCAKEKLRYFDWLSDAFDIWGQGTMVTVSSSEPKSSDKTHTCPPCP | |
| domain in | APEAAGAPSVFLFPPKPKDTLMISRTPEVTCVVVDVSHEDPEVKFNWYV | |
| italics)(CDR | DGVEVHNAKTKPREEQYNSTYRVVSVLTVLHQDWLNGKEYKCAVSNKAL | |
| sequences | PAPIEKTISKAKGQPREPQVYTLPPSRDELTKNQVSLTCLVKGFYPSDI | |
| are single- | AVEWESNGQPENNYKTTPPVLDSDGSFFLYSKLTVDKSRWQQGNVFSCS | |
| underlined) | VMHEALHNHYTQKSLSLSPGSGGGGSGGGGSGGGGSPSQVQLVQSGPEV | |
| | KKPGSSVKVSCKASGYTFSRSTMHWVRQAPGQGLEWIGYINPSSAYTNY | |
| | NQKFKDRVTITADKSTSTAYMELSSLRSEDTAVYYCARPQVHYDYNGFP | |
| | YWGQGTLVTVSSGGGGSGGGGSGGGGSGGGGSDIQMTQSPSTLSASVGD | |
| | RVTMTCSASSSVSYMNWYQQKPGKAPKRWIYDSSKLASGVPSRFSGSGS | |
| | GTDYTLTISSLQPDDFATYYCQQWSRNPPTFGGGTKVEIKRS | |

In certain embodiments, the CD123-binding domain comprises (i) an immunoglobulin light chain variable region (V_{L}) comprising CDRs LCDR1, LCDR2, and LCDR3, and (ii) an immunoglobulin heavy chain variable region (V_{H}) comprising CDRs HCDR1, HCDR2, and HCDR3 with HCDR1 comprising an amino acid sequence as set forth in SEQ ID NO:144, with HCDR2 comprising an amino acid sequence as set forth in SEQ ID NO:146 and with HCDR3 comprising an amino acid sequence as set forth in SEQ ID NO:148. In certain embodiments, the CD123-binding domain comprises (i) an immunoglobulin light chain variable region (VL) comprising CDRs LCDR1, LCDR2, and LCDR3, and (ii) an immunoglobulin heavy chain variable region (VH) comprising CDRs HCDR1, HCDR2, and HCDR3. In some such embodiments, (i) the LCDR1 has an amino acid sequence set forth in SEQ ID NO:138 or a sequence that differs from SEQ ID NO:138 by at least one amino acid substitution; (ii) the LCDR2 has an amino acid sequence set forth in SEQ ID NO:140 or a sequence that differs from SEQ ID NO:140 by at least one amino acid substitution; (iii) the LCDR3 has an amino acid sequence set forth in SEQ ID NO:142 or a sequence that differs from SEQ ID NO:142 by at least one amino acid substitution; (iv) the HCDR1 has an amino acid sequence set forth in SEQ ID NO:144 or a sequence that differs from SEQ ID NO:144 by at least one amino acid substitution; (v) the HCDR2 has an amino acid sequence set forth in SEQ ID NO:146 or a sequence that differs from SEQ ID NO:146 by at least one amino acid substitution; and (vi) the HCDR3 has an amino acid sequence set forth in SEQ ID NO:148 or a sequence that differs from SEQ ID NO:148 by at least one amino acid substitution. The amino acid substitution described above may be a conservative or a non-conservative amino acid substitution. In some embodiments, an LCDR1, LCDR2, LCDR3, HCDR1, HCDR2, and/or HCDR3 differs from a recited sequence by 1, 2, 3, 4, 5, 6, 7, 8, 9, or 10 amino acids. In certain embodiments, a CDR of the present disclosure contains about one or more (e.g., about 2, 3, 4, 5, 6, 7, 8, 9, 10) insertions, about one or more (e.g., about 2, 3, 4, 5, 6, 7, 8, 9, 10) deletions, about one or more (e.g., about 2, 3, 4, 5, 6, 7, 8, 9, 10) amino acid substitutions (e.g., conservative amino acid substitutions or non-conservative amino acid substitutions), or a combination of the above-noted changes, when compared to the CDR sequence of a known monoclonal antibody. For instance, the disclosure includes a recombinant polypeptide comprising (i) the LCDR1 has an amino acid sequence set forth in SEQ ID NO:138 or a sequence that differs from SEQ ID NO:138 by one or two amino acid substitutions; (ii) the LCDR2 has an amino acid sequence set forth in SEQ ID NO:140 or a sequence that differs from SEQ ID NO:140 by one or two amino acid substitutions; (iii) the LCDR3 has an amino acid sequence set forth in SEQ ID NO:142 or a sequence that differs from SEQ ID NO:142 by one or two amino acid substitutions; (iv) the HCDR1 has an amino acid sequence set forth in SEQ ID NO:144 or a sequence that differs from SEQ ID NO:144 by one or two amino acid substitutions; (v) the HCDR2 has an amino acid sequence set forth in SEQ ID NO:146 or a sequence that differs from SEQ ID NO:146 by one or two amino acid substitutions; and (vi) the HCDR3 has an amino acid sequence set forth in SEQ ID NO:148 or a sequence that differs from SEQ ID NO:148 by one or two amino acid substitutions. The amino acid substitution described above may be a conservative or a non-conservative amino acid substitution.

In related embodiments, a recombinant polypeptide of the disclosure comprises or is a sequence that is at least about 80%, at least about 85%, at least about 88%, at least about 90%, at least about 91%, at least about 92%, at least about 93%, at least about 94%, at least about 95%, at least about 96%, at least about 97%, at least about 98%, at least about 99%, at least about 99.5%, or 100% identical to an amino acid sequence of a light chain variable region (V_{L}) (e.g., SEQ ID NO:134) or to a heavy chain variable region (V_{H}) (e.g., SEQ ID NO:136), or both. In one embodiment, the CD123-binding domain of the recombinant polypeptide is an scFv comprising a variable heavy chain comprising SEQ ID NO:136 and a variable light chain comprising SEQ ID NO:134 in the VHVL orientation. In another embodiment, the CD123-binding domain of the recombinant polypeptide is an scFv comprising a variable light chain comprising SEQ ID NO:134 and a variable heavy chain comprising SEQ ID NO:136 in the VLVH orientation. For instance, in certain embodiments, the polypeptide of the disclosure comprises an amino acid sequence of SEQ ID NO:337. The instant disclosure includes a recombinant polypeptide that is at least about 80%, at least about 85%, at least about 88%, at least about 90%, at least about 91%, at least about 92%, at least about 93%, at least about 94%, at least about 95%, at least about 96%, at least about 97%, at least about 98%, at least about 99%, at least about 99.5%, or 100% identical to an amino acid sequence of SEQ ID NO:337.

In certain embodiments, the CD123-binding domain comprises (i) an immunoglobulin light chain variable region (V_{L}) comprising CDRs LCDR1, LCDR2, and LCDR3, and (ii) an immunoglobulin heavy chain variable region (V_{H}) comprising CDRs HCDR1, HCDR2, and HCDR3. In some such embodiments, (i) the LCDR1 has an amino acid sequence set forth in SEQ ID NO:154 or a sequence that differs from SEQ ID NO:154 by at least one amino acid substitution; (ii) the LCDR2 has an amino acid sequence set forth in SEQ ID NO:156 or a sequence that differs from SEQ ID NO:156 by at least one amino acid substitution; (iii) the LCDR3 has an amino acid sequence set forth in SEQ ID NO:158 or a sequence that differs from SEQ ID NO:158 by at least one amino acid substitution; (iv) the HCDR1 has an amino acid sequence set forth in SEQ ID NO:160 or a sequence that differs from SEQ ID NO:160 by at least one amino acid substitution; (v) the HCDR2 has an amino acid sequence set forth in SEQ ID NO:162 or a sequence that differs from SEQ ID NO:162 by at least one amino acid substitution; and (vi) the HCDR3 has an amino acid sequence set forth in SEQ ID NO:164 or a sequence that differs from SEQ ID NO:164 by at least one amino acid substitution. The amino acid substitution described above may be a conservative or a non-conservative amino acid substitution. In some embodiments, an LCDR1, LCDR2, LCDR3, HCDR1, HCDR2, and/or HCDR3 differs from a recited sequence by 1, 2, 3, 4, 5, 6, 7, 8, 9, or 10 amino acids. In certain embodiments, a CDR of the present disclosure contains about one or more (e.g., about 2, 3, 4, 5, 6, 7, 8, 9, 10) insertions, about one or more (e.g., about 2, 3, 4, 5, 6, 7, 8, 9, 10) deletions, about one or more (e.g., about 2, 3, 4, 5, 6, 7, 8, 9, 10) amino acid substitutions (e.g., conservative amino acid substitutions or non-conservative amino acid substitutions), or a combination of the above-noted changes, when compared to the CDR sequence of a known monoclonal antibody.

In related embodiments, a CD123-binding domain comprises or is a sequence that is at least about 80%, at least about 85%, at least about 88%, at least about 90%, at least about 91%, at least about 92%, at least about 93%, at least about 94%, at least about 95%, at least about 96%, at least about 97%, at least about 98%, at least about 99%, at least about 99.5%, or 100% identical to an amino acid sequence of a light chain variable region (V_{L}) (e.g., SEQ ID NO:17) or to a heavy chain variable region (V_{H}) (e.g., SEQ ID NO: 16), or both.

In certain embodiments, a CD123-binding domain comprises humanized immunoglobulin V_{L} and/or V_{H} regions. Techniques for humanizing immunoglobulin V_{L} and V_{H} regions are known in the art and are discussed, for example, in U.S. Patent Application Publication No. 2006/0153837. In certain embodiments, a CD123-binding domain comprises human immunoglobulin V_{L} and/or V_{H} regions.

Essentially, humanization by CDR grafting involves recombining only the CDRs of a non-human antibody onto a human variable region framework and a human constant region. Theoretically, this should substantially reduce or eliminate immunogenicity (except if allotypic or idiotypic differences exist). However, it has been reported that some framework residues of the original antibody also may need to be preserved (Reichmann et al., Nature, 332:323 (1988); Queen et al., Proc. Natl. Acad. Sci. USA, 86:10,029 (1989)).

The framework residues that need to be preserved are amenable to identification through computer modeling. Alternatively, critical framework residues can potentially be identified by comparing known antigen-binding site structures (Padlan, Molec. Immunol., 31(3):169-217 (1994), incorporated herein by reference).

The residues that potentially affect antigen binding fall into several groups. The first group comprises residues that are contiguous with the antigen site surface, which could therefore make direct contact with antigens. These residues include the amino-terminal residues and those adjacent to the CDRs. The second group includes residues that could alter the structure or relative alignment of the CDRs, either by contacting the CDRs or another peptide chain in the antibody. The third group comprises amino acids with buried side chains that could influence the structural integrity of the variable domains. The residues in these groups are usually found in the same positions (Padlan, 1994, supra) although their positions as identified may differ depending on the numbering system (see Kabat et al., “Sequences of proteins of immunological interest, 5th ed., Pub. No. 91-3242, U.S. Dept. Health & Human Services, NIH, Bethesda, Md., 1991).

Knowledge about humanized antibodies in the art is applicable to the polypeptides according to the disclosure, even if these polypeptides are not antibodies.

In some embodiments, an anti-CD123 scFv comprises a HCDR1 that comprises SEQ ID NO: 10, a HCDR2 that comprises SEQ ID NO: 11, and a HDCR3 that comprises SEQ ID NO: 12; and a LCDR1 that comprises SEQ ID NO: 13, a LCDR2 that comprises SEQ ID NO: 14, and a LCDR3 that comprises SEQ ID NO: 15. In some embodiments, the anti-CD123 scFv comprises a VH comprising a sequence at least 90%, at least 95%, or 100% identical to SEQ ID NO: 136, and a VL comprising a sequence at least 90%, at least 95%, or 100% identical to SEQ ID NO: 134. In some embodiments, the anti-CD123 scFv comprises a VH comprising a sequence at least 90%, at least 95%, or 100% identical to SEQ ID NO: 16. In some embodiments, the anti-CD123 scFv comprises a VL comprising a sequence at least 90%, at least 95%, or 100% identical to SEQ ID NO: 17. In some embodiments, the tumor antigen binding domain is an anti-CD123 scFv, and wherein the scFv comprises a sequence at least 90%, at least 95%, or 100% identical to SEQ ID NO: 18.

In some embodiments, the disclosure relates to CD123-binding domains wherein (i) the immunoglobulin light chain variable region comprises an amino acid sequence that is at least 88%, at least 90%, at least 92%, at least 95%, at least 97%, at least 98% or at least 99% identical to the amino acid sequence set forth in SEQ ID NO:134 and the immunoglobulin heavy chain variable region comprises an amino acid sequence that is at least 85%, at least 90%, at least 92%, at least 95%, at least 97%, at least 98% or at least 99% identical to the amino acid sequence set forth in SEQ ID NO:136.

In further embodiments, each CDR comprises no more than one, two, or three substitutions, insertions or deletions, as compared to that from a monoclonal antibody or fragment or derivative thereof that specifically binds to a target of interest (e.g., CD123).

In certain embodiments, a CD123-binding domain does not inhibit IL-3 binding to CD123.

In certain embodiments, a CD123-binding molecule or protein can comprise a T-cell binding domain for recruitment of T-cells to target cells expressing CD123. In certain embodiments, a CD123-binding protein as described herein can comprise (i) a binding domain that specifically binds a TCR complex or a component thereof (e.g., TCRα, TCRβ, CD3γ, CD3δ, and CD3ε) and (ii) another binding domain that specifically binds to CD123. A CD123-binding protein can utilize essentially any binding domain that binds a T-cell, e.g., an antibody derived binding domain. Exemplary anti-CD3 antibodies from which the CD3 binding domain can be derived include the CRIS-7 monoclonal antibody (Reinherz, E. L. et al. (eds.), Leukocyte typing II., Springer Verlag, New York, (1986); V_{L} and V_{H} amino acid sequences respectively shown in SEQ ID NO: 341 (QVVLTQSPAIMSAFPGEKVTMTCSASSSVSYMNWYQQKSGTSPKRWIYDSSKLA SGVPARFSGSGSGTSYSLTISSMETEDAATYYCQQWSRNPPTFGGGTKLQITR) and SEQ ID NO: 342 (QVQLQQSGAELARPGASVKMSCKASGYTFTRSTMHWVKQRPGQGLEWIGYINP SSAYTNYNQKFKDKATLTADKSSSTAYMQLSSLTSEDSAVYYCASPQVHYDYNGF PYWGQGTLVTVSA)); HuM291 (Chau et al. (2001) Transplantation 71:941-950; V_{L} and V_{H} amino acid sequences respectively shown in SEQ ID NO:343 (DIQMTQSPSSLSASVGDRVTITCSASSSVSYMNWYQQKPGKAPKRLIYDTSKLAS GVPSRFSGSGSGTDFTLTISSLQPEDFATYYCQQWSSNPPTFGGGTKVEIK) and SEQ ID NO: 344 (QVQLVQSGAEVKKPGASVKVSCKASGYTFISYTMHWVRQAPGQGLEWMGYINPR SGYTHYNQKLKDKATLTADKSASTAYMELSSLRSEDTAVYYCARSAYYDYDGFAY WGQGTLVTVSS)); BC3 monoclonal antibody (Anasetti et al. (1990) J. Exp. Med. 172:1691); OKT3 monoclonal antibody (Ortho multicenter Transplant Study Group (1985) N. Engl. J. Med. 313:337) and derivatives thereof such as OKT3 ala-ala (also referred to as OKT3 AA-FL or OKT3 FL), a humanized, Fc variant with alanine substitutions at positions 234 and 235 (Herold et al. (2003) J. Clin. Invest. 11:409); visilizumab (Carpenter et al. (2002) Blood 99:2712), G19-4 monoclonal antibody (Ledbetter et al., 1986, J. Immunol. 136:3945), 145-2C11 monoclonal antibody (Hirsch et al. (1988) J. Immunol. 140: 3766) and I2C monoclonal antibody (see, e.g., US 2011/0293619 and US20120244162). For example, a CD3 binding domain may comprise a CD3 binding domain disclosed in U.S. Patent Application Publication No. 2012/0244162, including a CD3 binding domain comprising a VL region selected from SEQ ID NO: 17, 21, 35, 39, 53, 57, 71, 75, 89, 83, 107, 111, 125, 129, 143, 147, 161, 165, 179 and 183 of US 2012/0244162 and/or a VH region selected from SEQ ID NO:15, 19, 33, 37, 51, 55, 69, 73, 87, 91. 105, 109, 123, 127, 141, 145, 159, 163, 177 and 181 of US 2012/0244162. In some embodiments, a CD3 binding domain comprises an amino acid sequence selected from SEQ ID NO: 23, 25, 41, 43, 59, 61, 77, 79, 95, 97, 113, 115, 131, 133, 149, 151, 167, 169, 185, and 187 of US 2012/0244162. In some embodiments, a CD3 binding domain is one described in WO2004/106380, WO2005/040220A1, US 2014/0099318 or derived from a CD3 binding domain thereof. An exemplary anti-TCR antibody is the BMA031 monoclonal antibody (Borst et al. (1990) Human Immunology 29:175-188). The CD3 binding domain may be derived from any of the antibodies or sequences described in WO 2013/158856 (incorporated herein by reference in its entirety).

In some embodiments, the second binding domain of a CD123-binding polypeptide described herein comprises: (i) an immunoglobulin light chain variable region comprising LCDR1, LCDR2, and LCDR3, and (ii) an immunoglobulin heavy chain variable region comprising HCDR1, HCDR2, and HCDR3, wherein (a) the LCDR1, LCDR2 and LCDR3 has the amino acid sequences set forth in SEQ ID NOs:348, 349 and 350, respectively, and the HCDR1, HCDR2, and HCDR3 has the amino acid sequences set forth in SEQ ID NOs: 345, 346 and 347, respectively; or (b) the LCDR1, LCDR2 and LCDR3 has the amino acid sequences set forth in SEQ ID NO:354, SEQ ID NO:355, and SEQ ID NO:356, respectively, and the HCDR1, HCDR2, and HCDR3 has the amino acid sequences set forth in SEQ ID NO: 351, SEQ ID NO:352, and SEQ ID NO:353, respectively. In some embodiments, the second binding domain of a CD123-binding polypeptide described herein comprises: (i) an immunoglobulin light chain variable region comprising LCDR1, LCDR2, and LCDR3, and (ii) an immunoglobulin heavy chain variable region comprising HCDR1, HCDR2, and HCDR3, wherein (a) the LCDR1, LCDR2 and LCDR3 has the amino acid sequences set forth in SEQ ID NOs: 182, 183 and 184, respectively, and the HCDR1, HCDR2, and HCDR3 has the amino acid sequences set forth in SEQ ID NOs: 351, 352 and 353, respectively, and the HCDR1, HCDR2, and HCDR3 has the amino acid sequences set forth in SEQ ID NOs: 357, 359 and 359, respectively; or (b) the LCDR1, LCDR2 and LCDR3 has the amino acid sequences set forth in SEQ ID NOs: 359, 367 and 368, respectively, and the HCDR1, HCDR2, and HCDR3 has the amino acid sequences set forth in SEQ ID NOs: 363, 364 and 365, respectively. In some embodiments, the second binding domain of a CD123-binding polypeptide described herein comprises: (i) an immunoglobulin light chain variable region comprising LCDR1, LCDR2, and LCDR3, and (ii) an immunoglobulin heavy chain variable region comprising HCDR1, HCDR2, and HCDR3, wherein (a) the LCDR1, LCDR2 and LCDR3 has the amino acid sequences set forth in SEQ ID NOs: 372, 373 and 374, respectively; or (b) the LCDR1, LCDR2 and LCDR3 has the amino acid sequences set forth in SEQ ID NOs: 378, 379 and 380, respectively, and the HCDR1, HCDR2, and HCDR3 has the amino acid sequences set forth in SEQ ID NOs: 375, 376 and 377, respectively. In some embodiments, the second binding domains comprising the CDR sequences recited in this paragraph are humanized.

In some embodiments of a CD123-binding protein comprising a second binding domain that specifically binds CD3ε, the second binding domain competes for binding to CD3ε with the CRIS-7, HuM291 or I2C monoclonal antibody. In some embodiments, the CD3-binding domain comprises an immunoglobulin light chain variable region (V_{L}) and an immunoglobulin heavy chain variable region (V_{H}) derived from the CRIS-7, HuM291 or I2C monoclonal antibody (e.g., the V_{L} and V_{H} of the second binding domain can be humanized variable regions comprising, respectively, the light chain CDRs and the heavy chain CDRs of the monoclonal antibody). A second binding domain may comprise the light chain variable region, the heavy chain variable region, or both, of the DRA222, TSC455, or TSC456 CD3-binding domains. The amino acid sequences of DRA222, TSC455, and TSC456 are provided in Table 4. The DRA222 binding domains are also described in WO 2013/158856. TSC455 may also be referred to as TSC394 F87Y. TSC455 may also be referred to as TSC394 E86D F87Y or TSC394 DY.

In some embodiments, the second binding domain specifically binds CD3 and comprises an immunoglobulin light chain variable region and an immunoglobulin heavy chain variable region; wherein the immunoglobulin light chain variable region comprises an amino acid sequence that is at least about 93% identical, at least about 95% identical, at least about 97% identical, at least about 98% identical or at least about 99% identical to the amino acid sequence in SEQ ID NO:384; or at least about 94% identical, at least about 95% identical, at least about 97% identical, at least about 98% identical or at least about 99% identical to the amino acid sequence in SEQ ID NO:385; and wherein the immunoglobulin heavy chain variable region comprises an amino acid sequence that is at least about 82% identical, at least about 85% identical, at least about 87% identical, at least about 90% identical, at least about 92% identical, at least about 95% identical, at least about 97% identical, at least about 98% identical or at least about 99% identical to the amino acid sequence in SEQ ID NO:383.

In some embodiments, the second binding domain is a CD3 binding domain that comprises a HCDR1 that comprises SEQ ID NO: 19, a HCDR2 that comprises SEQ ID NO: 20, and a HDCR3 that comprises SEQ ID NO: 21; and a LCDR1 that comprises SEQ ID NO: 22, a LCDR2 that comprises SEQ ID NO: 23, and a LCDR3 that comprises SEQ ID NO: 24. In some embodiments, the CD3 binding domain is an anti-CD3 scFv that comprises a VH comprising a sequence at least 90%, at least 95%, or 100% identical to SEQ ID NO: 383 or 387, and a VL comprising a sequence at least 90%, at least 95%, or 100% identical to SEQ ID NO: 384. In some embodiments, the CD3 binding domain comprises a VH comprising a sequence at least 90%, at least 95%, or 100% identical to SEQ ID NO: 25. In some embodiments, the CD3 binding domain comprises a VL comprising a sequence at least 90%, at least 95%, or 100% identical to SEQ ID NO: 26. In some embodiments, the CD3 binding domain is an anti-CD3 scFv that comprises a sequence at least 90%, at least 95%, or 100% identical to SEQ ID NO: 27.

In some embodiments, a CD123-binding polypeptide or protein further comprising a CD3-binding domain may have a low level of high molecular weight aggregates produced during recombinant expression of the polypeptide or protein. A CD123-binding polypeptide or protein further comprising a CD3-binding domain may exhibit a relatively long stability in human serum, depending on the CD3-binding domain present in the polypeptide or protein.

In certain variations, the CD3-binding domain and comprises one or more of the CD3-binding sequences (e.g., CDRs or variable regions) disclosed in US 2013/0129730, US 2011/0293619, U.S. Pat. No. 7,635,472, WO 2010/037836, WO 2004/106381, or WO 2011/121110; each incorporated herein by reference in its entirety. In some embodiments, a CD3-binding domain comprises one or more of the sequences shown in Table 6.

TABLE 6
Exemplary CD3-binding domain light chain CDRs

| LCDR1 | LCDR2 | LCDR3 |
|---|---|---|
| GSSTGAVTSGYYPN | GTKFLAP | ALWYSNRWV |
| (SEQ ID NO: 110) | (SEQ ID NO: | (SEQ ID NO: 116) |
| | 113) | |
| RSSTGAVTSGYYPN | ATDMRPS | ALWYSNRWV |
| (SEQ ID NO: 111) | (SEQ ID NO: | (SEQ ID NO: 117) |
| | 114) | |
| GSSTGAVTSGNYPN | GTKFLAP | VLWYSNRWV |
| (SEQ ID NO: 112) | (SEQ ID NO: | (SEQ ID NO: 118) |
| | 115) | |

In various embodiments, a CD3-binding domain comprises one or more of the sequences shown in Table 7.

TABLE 7
Exemplary CD3-binding domain heavy chain CDRs

| HCDR1 | HCDR2 | HCDR3 |
|---|---|---|
| IYAMN | RIRSKYNNYATYYADSVKS | HGNFGNSYVSFFAY |
| (SEQ ID NO: | (SEQ ID NO: 122) | (SEQ ID NO: 125) |
| 119) | | |
| KYAMN | RIRSKYNNYATYYADSVKD | HGNFGNSYISYWAY |
| (SEQ ID NO: | (SEQ ID NO: 123) | (SEQ ID NO: 126) |
| 120) | | |
| SYAMN | RIRSKYNNYATYYADSVKG | HGNFGNSYLSFWAY |
| (SEQ ID NO: | (SEQ ID NO: 124) | (SEQ ID NO: 127) |
| 121) | | |

In some embodiments, a therapeutic protein comprises, in order from amino terminus to carboxyl terminus a first binding domain, a hinge region, an immunoglobulin constant region, and a second binding domain. In some embodiments, the immunoglobulin constant region comprises immunoglobulin CH2 and CH3 domains of IgG1, IgG2, IgG3, IgG4, IgA1, IgA2 or IgD. In some embodiments, the first binding domain comprises: an immunoglobulin heavy chain variable region (VH) comprising HCDR1, HCDR2, and HCDR3; and an immunoglobulin light chain variable region (VL) comprising LCDR1, LCDR2, and LCDR3. In some embodiments, the HCDR1 comprises SEQ ID NO: 10, the HCDR2 comprises SEQ ID NO: 11, and the HDCR3 comprises SEQ ID NO: 12. In some embodiments, the LCDR1 comprises SEQ ID NO: 13, the LCDR2 comprises SEQ ID NO: 14, and the LCDR3 comprises SEQ ID NO: 15. In some embodiments, the HCDR1 comprises SEQ ID NO: 10, the HCDR2 comprises SEQ ID NO: 11, and the HDCR3 comprises SEQ ID NO: 12; and the LCDR1 comprises SEQ ID NO: 13, the LCDR2 comprises SEQ ID NO: 14, and the LCDR3 comprises SEQ ID NO: 15. In some embodiments, the first binding domain comprises a sequence at least 95% identical to SEQ ID NO: 18. In some embodiments, the second binding domain comprises an immunoglobulin heavy chain variable region (VH) comprising HCDR1, HCDR2, and HCDR3; and (ii) an immunoglobulin light chain variable region (VL) comprising LCDR1, LCDR2, and LCDR3. In some embodiments, the HCDR1 comprises SEQ ID NO: 19, the HCDR2 comprises SEQ ID NO: 20, and the HDCR3 comprises SEQ ID NO: 21. In some embodiments, the LCDR1 comprises SEQ ID NO: 22, the LCDR2 comprises SEQ ID NO: 23, and the LCDR3 comprises SEQ ID NO: 24. In some embodiments, the HCDR1 comprises SEQ ID NO: 19, the HCDR2 comprises SEQ ID NO: 20, and the HDCR3 comprises SEQ ID NO: 21; and the LCDR1 comprises SEQ ID NO: 22, the LCDR2 comprises SEQ ID NO: 23, and the LCDR3 comprises SEQ ID NO: 24. In some embodiments, the second binding domain comprises a sequence at least 95% or 100% identical to SEQ ID NO: 27. In some embodiments, the therapeutic protein comprises the sequence of SEQ ID NO: 31.

The structural format of multispecific anti-CD123 and anti-CD3 molecules disclosed herein induces potent tumor cell lysis but reduced cytokine release compared to multispecific anti-CD123 and anti-CD3 molecules in alternative structural formats. Without being bound by any theory, the polypeptide structural format disclosed herein (e.g., in order from amino terminus to carboxyl terminus): (a) a first binding domain that is a CD123-binding domain; (b) a hinge region; (c) an immunoglobulin constant region; and (d) a second binding domain that is a human or humanized binding domain that specifically binds a T-cell, CD3, CD3ε or a T-cell receptor (TCR) complex) induces a moderate level of T-cell Receptor (TCR) stimulation, compared to other T-cell engagers. It has been extensively documented that the strength or magnitude of the TCR signal regulates the outcome of T-cell activation. TCR stimulation triggers a number of cellular events that include initiation of effector function (e.g., cytolytic granzymes), and cytokine secretion and cell division (Corse, Gottschalk and Allison. J Immunol 2011, 186:5039-5045). These distinct cellular events can proceed with different kinetics and reach variable maximum levels, depending on the intensity of the TCR stimulus and additional factors. The multispecific structural format disclosed herein is sufficiently potent to cause lysis of tumor cells over multiple days and to induce multiple rounds of T-cell division but moderate enough to limit the amount of cytokine secretion.

In some embodiments, the multispecific polypeptide comprising a CD123-binding domain and a CD3-binding domain when bound to a CD3 protein on a T cell induces reduced cytokine release from said T cell as compared to an OKT3 antibody control. In some embodiments, the multispecific polypeptide comprising a CD123-binding domain and CD3-binding domain induces reduced cytokine release from said T cell as compared to a multispecific polypeptide comprising an CD3-binding domain derived from OKT3 or I2C. In some embodiments, the multispecific polypeptide comprising a CD123-binding domain (e.g., a CD123-binding domain comprising an amino acid sequence at least 93%, at least 95%, at least 96%, at least 97%, at least 98%, at least 99% or 100% identical to SEQ ID NO: 312 and/or SEQ ID NO:337) and a CD3-binding domain and in the scFv-Fc-scFv format induces reduced cytokine release in a non-human primate or human as compared to a bispecific polypeptide comprising a CD123-binding domain and I2C derived CD3-binding domain in a bispecific T-cell engager (scFv-scFv) format or dual affinity re-targeting format.

Also provided herein are pharmaceutical compositions comprising the therapeutic proteins described herein. In some embodiments, the compositions comprise 1-20 mg/m, 2.5-12 mg/ml, or 5-10 mg/ml of a therapeutic protein. In some embodiments, the compositions comprise from about 2.5 mg/ml to about 12 mg/ml, or from about 5 mg/ml to about 10 mg/ml of a therapeutic protein. In some embodiments, the compositions comprise about 1, about 2, about 3, about 4, about 5, about 6, about 7, about 8, about 9, about 10, about 11, or about 12 mg/ml of a therapeutic protein. In some embodiments, the compositions comprise about 5 mg/ml of a therapeutic protein.

### Methods of Use

The present disclosure provides methods for treating a subject with a disease or disorder, the methods comprising administering a therapeutically effective amount of at least one composition of the disclosure to the subject.

In some embodiments, the disease or disorder may be cancer. The cancer may be selected from, for example, acute myeloid leukemia (AML), myelodysplastic syndrome (MDS), hairy cell leukemia (HCL), blastic plasmacytoid dendritic cell neoplasm, B-cell acute lymphoblastic leukemia (ALL), and chronic myeloid leukemia (CML).

In some embodiments, the disease or disorder may be an inflammatory disease or disorder. In embodiments, the inflammatory disease or disorder may be an autoimmune disease or disorder. In some embodiments, the autoimmune disease or disorder is selected from irritable bowel syndrome, inflammatory bowel disease (e.g. Crohn's disease or ulcerative colitis), psoriasis, rheumatoid arthritis, juvenile rheumatoid arthritis, psoriatic arthritis, systemic lupus erythematosus, asthma, multiple sclerosis, dermatomyositis, polymyositis, pernicious anaemia, primary biliary cirrhosis, acute disseminated encephalomyelitis (ADEM), Addison's disease, ankylosing spondylitis, antiphospholipid antibody syndrome (aPL), autoimmune hepatitis, diabetes mellitus type 1, Goodpasture's syndrome, Graves' disease, Guillain-Barré syndrome (GBS), Hashimoto's disease, idiopathic thrombocytopenic purpura, pemphigus vulgaris, Sjögren's syndrome, temporal arteritis, autoimmune hemolytic anemia, bullous pemphigoid, vasculitis, celiac disease, endometriosis, hidradenitis suppurativa, interstitial cystitis, morphea, scleroderma, narcolepsy, neuromyotonia, vitiligo, autoimmune inner ear disease and myasthenia gravis. In some embodiments, the inflammatory disease or disorder is psoriasis.

In some embodiments, the inflammatory disease or disorder may be a “neuroimmune disease” such as neuropathic pain, osteoarthritis, Parkinson's disease, amyotrophic lateral sclerosis, Huntington's disease, and Alzheimer's disease.

In some embodiments, the inflammatory disease or disorder may be an adverse transplant associated event, i.e. transplant rejection, allograft disease or graft-versus-host disease.

In some embodiments, for treatment methods and uses described herein, a protein or polypeptide described herein is delivered in a manner consistent with conventional methodologies associated with management of the disease or disorder for which treatment is sought. In accordance with the disclosure herein, a therapeutically effective amount of the protein or polypeptide is administered to a subject in need of such treatment for a time and under conditions sufficient to prevent or treat the disease or disorder.

Subjects for administration of a protein of the present disclosure include patients at high risk for developing a particular disorder as well as patients presenting with an existing such disorder. Typically, the subject has been diagnosed as having the disorder for which treatment is sought. Further, subjects can be monitored during the course of treatment for any change in the disorder (e.g., for an increase or decrease in clinical symptoms of the disorder). Also, in some variations, the subject does not suffer from another disorder requiring treatment.

In prophylactic applications, pharmaceutical compositions or medicants comprising a protein of the present disclosure are administered to a patient susceptible to, or otherwise at risk of, a particular disorder in an amount sufficient to eliminate or reduce the risk or delay the onset of the disorder. In therapeutic applications, compositions or medicants comprising a protein of the present disclosure are administered to a patient suspected of, or already suffering from such a disorder in an amount sufficient to cure, or at least partially arrest, the symptoms of the disorder and its complications. An amount adequate to accomplish this is referred to as a therapeutically effective dose or amount. In both prophylactic and therapeutic regimes, agents are usually administered in several dosages until a sufficient response (e.g., inhibition of inappropriate angiogenesis activity) has been achieved. Typically, the response is monitored and repeated dosages are given if the desired response starts to fade.

To identify subject patients for treatment according to the methods of the disclosure, accepted screening methods can be employed to determine risk factors associated with specific disorders or to determine the status of an existing disorder identified in a subject. Such methods can include, for example, determining whether an individual has relatives who have been diagnosed with a particular disorder. Screening methods can also include, for example, conventional work-ups to determine familial status for a particular disorder known to have a heritable component. For example, various cancers are also known to have certain inheritable components. Inheritable components of cancers include, for example, mutations in multiple genes that are transforming (e.g., Ras, Raf, EGFR, cMet, and others), the presence or absence of certain HLA and killer inhibitory receptor (KIR) molecules, or mechanisms by which cancer cells are able to modulate immune suppression of cells like NK cells and T-cells, either directly or indirectly (see, e.g., Ljunggren and Malmberg, Nature Rev. Immunol. 7:329-339, 2007; Boyton and Altmann, Clin. Exp. Immunol. 149:1-8, 2007). Toward this end, nucleotide probes can be routinely employed to identify individuals carrying genetic markers associated with a particular disorder of interest. In addition, a wide variety of immunological methods are known in the art that are useful to identify markers for specific disorder. For example, various ELISA immunoassay methods are available and well-known in the art that employ monoclonal antibody probes to detect antigens associated with specific tumors. Screening can be implemented as indicated by known patient symptomology, age factors, related risk factors, etc. These methods allow the clinician to routinely select patients in need of the methods described herein for treatment.

For administration, the pharmaceutical compositions of the disclosure may comprise: (i) therapeutic protein/polypeptide; and (ii) a pharmaceutically acceptable carrier, diluent or excipient. In some embodiments, the pharmaceutical composition may comprise (i) a therapeutic protein/peptide, (ii) a buffer, (iii) an excipient, and (iv) a surfactant.

A pharmaceutical composition comprising a polypeptide or protein described herein may be formulated in a dosage form selected from the group consisting of: an oral unit dosage form, an intravenous unit dosage form, an intranasal unit dosage form, a suppository unit dosage form, an intradermal unit dosage form, an intramuscular unit dosage form, an intraperitoneal unit dosage form, a subcutaneous unit dosage form, an epidural unit dosage form, a sublingual unit dosage form, and an intracerebral unit dosage form. The oral unit dosage form may be selected from the group consisting of: tablets, pills, pellets, capsules, powders, lozenges, granules, solutions, suspensions, emulsions, syrups, elixirs, sustained-release formulations, aerosols, and sprays.

A pharmaceutical composition comprising polypeptide or protein described herein may be administered to a subject in a therapeutically effective amount. According to the methods of the present disclosure, polypeptide or protein described herein can be administered to subjects by a variety of administration modes, including, for example, by intramuscular, subcutaneous, intravenous, intra-atrial, intra-articular, parenteral, intranasal, intrapulmonary, transdermal, intrapleural, intrathecal, and oral routes of administration. For prevention and treatment purposes, an antagonist can be administered to a subject in a single bolus delivery, via continuous delivery (e.g., continuous transdermal delivery) over an extended time period, or in a repeated administration protocol (e.g., on an hourly, daily, weekly, or monthly basis).

Determination of effective dosages in this context is typically based on animal model studies followed up by human clinical trials and is guided by determining effective dosages and administration protocols that significantly reduce the occurrence or severity of the subject disorder in model subjects. Effective doses of the compositions of the present disclosure vary depending upon many different factors, including means of administration, target site, physiological state of the patient, whether the patient is human or an animal, other medications administered, whether treatment is prophylactic or therapeutic, as well as the specific activity of the composition itself and its ability to elicit the desired response in the individual. Usually, the patient is a human, but in some diseases, the patient can be a nonhuman mammal. Typically, dosage regimens are adjusted to provide an optimum therapeutic response, i.e., to optimize safety and efficacy.

Also provided herein are uses of the compositions of the disclosure in the manufacture of a medicament for treating cancer. For instance, compositions of the disclosure may be used for treatment of acute myeloid leukemia (AML) or myelodysplastic syndrome (MDS). Also provided are methods comprising administering a composition comprising a multispecific polypeptide comprising a CD123 binding domain and a CD3 binding domain to a patient by IV infusion at a weekly dose of about 0.3, about 1, about 3, about 6, about 9, about 12, about 18, about 20, about 24, about 30, about 36, about 50, about 48, about 60, about 75, or about 100 μg. Typically a patient is treated once or twice a week for 4 to 6 weeks. The patient may receive the same dosage each week or the dosage may be increased, for instance, each week.

In some embodiments, the dosage is increased each week, with the first dosage being less that what a patient would be expected to tolerate. This type of step-up treatment regimen reduces the risk that the patient will develop an infusion related reaction or cytokine release syndrome. In some embodiments, a multispecific protein comprising a CD123 binding domain and a CD3 binding domain (e.g., TRI130 or TRI129) may be administered to a patient intravenously such that the dosage is increased each week for at least the first two or first three doses. For instance, a composition of the disclosure may be administered by IV infusion according to the following weekly treatment schedule: week 1 dosage: 6 μg; week 2 dosage: 9 μg; week 3 dosage: 12 μg; and week 4 dosage and subsequent week dosages: 12 μg. In some embodiments, a patient may be administered a composition of the disclosure intravenously according to the following weekly treatment schedule: week 1 dosage: 6 μg; week 2 dosage: 9 μg; week 3 dosage: 12 μg; and week 4 dosage and subsequent week dosages: 18 μg. In some embodiments, the composition is administered to a patient intravenously according to the weekly treatment schedule: week 1 dosage: 6 μg; and week 2 and subsequent week dosages: 9 μg, and in some embodiments, the composition is administered to a patient intravenously according to the weekly treatment schedule week 1 dosage: 9 μg; and week 2 and subsequent week dosages: 12 μg. In other embodiments, the composition is administered to a patient intravenously according to the weekly treatment schedule: week 1 dosage 12 μg, and week 2 and subsequent week dosages: 18 μg.

In some embodiments, a patient may be administered a composition of the disclosure intravenously according to the following weekly treatment schedule: week 1 dosage: 6 μg; week 2 dosage: 9 μg; week 3 dosage: 12 μg; week 4 dosage, and subsequent week doses: 12 μg. In some embodiments, a patient may be administered a composition of the disclosure intravenously according to the following weekly treatment schedule: week 1 dosage: 6 μg; week 2 dosage: 9 μg; week 3 dosage: 12 μg; week 4 dosage, and subsequent week doses: 18 μg. In some embodiments, a patient may be administered a composition of the disclosure intravenously according to the following weekly treatment schedule: week 1 dosage: 6 μg; week 2 dosage: 12 μg; week 3 dosage: 12 μg; week 4 dosage, and subsequent week doses: 12 μg. In some embodiments, a patient may be administered a composition of the disclosure intravenously according to the following weekly treatment schedule: week 1 dosage: 6 μg; week 2 dosage: 12 μg; week 3 dosage: 18 μg; week 4 dosage, and subsequent week doses: 24 μg. In some embodiments, a patient may be administered a composition of the disclosure intravenously according to the following weekly treatment schedule: week 1 dosage: 6 μg; week 2 dosage: 12 μg; week 3 dosage: 18 μg; week 4 dosage, and subsequent week doses: 36 μg. In some embodiments, a patient may be administered a composition of the disclosure intravenously according to the following weekly treatment schedule: week 1 dosage: 6 μg; week 2 dosage: 12 μg; week 3 dosage: 18 μg; week 4 dosage, and subsequent week doses: 48 μg. In some embodiments, a patient may be administered a composition of the disclosure intravenously according to the following weekly treatment schedule: week 1 dosage: 6 μg; week 2 dosage: 12 μg; week 3 dosage: 18 μg; week 4 dosage, and subsequent week doses: 60 μg.

In some embodiments, a patient may be administered a composition of the disclosure intravenously according to the following treatment schedule: day 1: 6 μg; day 2: 9 μg; day 3:12 μg; day 4:18 μg; day 8:18 μg; day 11: 18 μg; day 15: 36 μg; day 22: 36 μg; followed by weekly doses of 36 μg.

In some embodiments, a patient may be administered a composition of the disclosure intravenously according to the following treatment schedule: day 1: 6 μg; day 2: 12 μg; day 3: 18 μg; day 4: 24 μg; day 8:24 μg; day 11: 24 μg; day 15: 48 μg; day 22: 48 μg; followed by weekly doses of 48 μg.

In some embodiments, a patient may be administered a composition of the disclosure intravenously according to the following treatment schedule: day 1: 6 μg; day 2: 12 μg; day 3: 24 μg; day 4: 36 μg; day 8: 36 μg; day 11: 36 μg; day 15: 60 μg; day 22: 60 μg, followed by weekly doses of 60 μg.

In some embodiments, a patient may be administered a composition of the disclosure intravenously according to the following treatment schedule: day 1: 6 μg; day 2: 12 μg; day 3:24 μg; day 4:36 μg; day 8:48 μg; day 11:48 μg; day 15: 100 μg; day 22: 100 μg, followed by weekly doses of 100 μg.

In some embodiments, a method for treating a patient in need thereof comprises administering a composition comprising a multispecific protein comprising a CD123 binding domain and a CD3 binding domain to the patient on days 1, 8, 15, and 22. In some embodiments, 6 μg is administered on day 1, 9 μg is administered on day 8, 12 μg is administered on day 15, and 12 μg is administered on day 22. In some embodiments, 6 μg is administered on day 1, 9 μg is administered on day 8, 12 μg is administered on day 15, and 18 μg is administered on day 22. In some embodiments, 6 μg is administered on day 1, 9 μg is administered on day 8, 9 μg is administered on day 15, and 9 μg is administered on day 22. In some embodiments, 9 μg is administered on day 1, 12 μg is administered on day 8, 12 μg is administered on day 15, and 12 μg is administered on day 22. In some embodiments, 12 μg is administered on day 1, 18 μg is administered on day 8, 18 μg is administered on day 15, and 18 μg is administered on day 22.

In some embodiments, a patient treated according to the methods of the disclosure exhibits a decrease in bone marrow blast percentage, and in some embodiments, a patient exhibits a decrease in absolute blast counts in the blood. In some embodiments, the treatment results in reduction in patient blast levels by at least 0.5%, at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50% or more, compared to the patient's levels immediately before the treatment.

In some embodiments, a patient treated according to the methods of the disclosure exhibits a complete remission (CR). As used herein, complete remission refers to a reduction in bone marrow blasts to less than about 5%, absence of circulating blasts and blasts with Auer rods, absence of extramedullary disease, and absolute neutrophil count (ANC)≥1.0×10⁹/L (1,000/μL) and PLT≥100×10⁹/L (100,000/μL). In some embodiments, a patient treated according to the methods of the disclosure exhibits a CR without Minimal Residual Disease (CR_{MRD}). As used herein, CR_{MRD} refers to CR with negativity for a genetic marker by quantitative reverse transcription polymerase chain reaction (RT-qPCR) or CR with negativity by multiparameter flow cytometry. In some embodiments, a patient treated according to the methods of the disclosure exhibits a CR with Incomplete Hematologic Recovery (CRᵢ). CRᵢ includes all the criteria of CR, described above, except for residual neutropenia (ANC<1.0×10⁹/L [1,000/μL]) or thrombocytopenia (PLT<100×10⁹/L [100,000/μL]).

In some embodiments, a patient treated according to the methods of the disclosure exhibits a Morphologic Leukemia-Free State (MLFS). As used herein, MLFS refers to bone marrow blasts<5% (i.e., marrow should not be merely “aplastic;” at least 200 cells should be enumerated or cellularity should be at least 10%); absence of blasts with Auer rods; and absence of extramedullary disease. No hematologic recovery is required.

In some embodiments, a patient treated according to the methods of the disclosure exhibits a Partial Remission (PR). As used herein, a PR includes all hematologic criteria of CR, described above, plus a decrease of bone marrow blast percentage to 5 to 25%, and at least 50% decrease of pretreatment bone marrow blast percentage.

In some embodiments, a patient treated according to the methods of the disclosure exhibits Stable Disease (SD), characterized by an absence of CR_{MRD}, CR, CRᵢ, PR, and MLFS, but without progressive disease (i.e., increase in bone marrow blast percentage and/or increase of absolute blast counts in the blood).

A patient treated with the CD123×CD3 targeting multispecific polypeptide (e.g., TRI130 or TRI129) at either the same dosage each week or with a step-up treatment regimen may also have infusion times (i.e., the length of the infusion) modified to further reduce the likelihood of an infusion reaction or cytokine release syndrome. To reduce the risk of an adverse event, the first dose is administered by IV to the patient over several hours, e.g., 20-24 hours. In some embodiments, the first dose of the composition is administered over a period of about 20-24 hours, the second dose is administered over a period of about 8 hours, the third dose is administered over a period of about 6 hours, and the fourth dose and subsequent doses are administered over a period of about 4 hours. In some embodiments, the first dose of the composition is administered over a period of about 20-24 hours, the second dose is administered over a period of about 8 hours, the third dose is administered over a period of about 6 hours, and the fourth dose and subsequent doses are administered over a period of about 4 hours, wherein each of the first, second, third, and fourth dose are the same. The composition can also be administered to a subject by continuous IV infusion, e.g., continuous IV infusion up to about 72 hours in duration.

A patient treated with the CD123×CD3 targeting multispecific polypeptide (e.g., TRI130 or TRI129) may also be treated with one or more additional therapeutic agents. The one or more additional therapeutic agents may be administered at or around the same time as the CD123×CD3 targeting multispecific polypeptide. In some embodiments, the one or more additional therapeutic agents are administered before (i.e., as a “premedication”) administration of the multispecific polypeptide, such as about 1-3 hours before administration thereof. In some embodiments, the one or more additional therapeutic agents are administered after administration of one or more doses of the multispecific polypeptide.

In some embodiments, the one or more additional therapeutic agents are diphenhydramine, acetaminophen, and/or dexamethasone. In some embodiments, the one or more additional therapeutic agents may be administered intravenously or orally. In some embodiments, dexamethasone may be administered at a dose of about 10 to about 20 mg. In some embodiments, methylprednisolone may be administered at a dose of about 1 mg/kg. In some embodiments, acetaminophen may be administered at a dose of about 650 or about 1,000 mg. In some embodiments, the acetaminophen may be administered three times a day for 1 day, with the first dose administered 1 to 3 hours before administration of the CD123×CD3 targeting multispecific polypeptide. In some embodiments, the one or more additional therapeutic agents may comprise an antihistamine such as diphenhydramine. Diphenhydramine may be administered at a dose of about 50 mg. In some embodiments, the one or more therapeutic agents may comprise allopurinol. In some embodiments, allopurinol is administered at least 2 days prior to administration of the CD123×CD3 targeting multispecific polypeptide. In some embodiments, the one or more additional therapeutic agents may comprise tocilizumab.

In some embodiments, a method for treating a disorder characterized by overexpression of CD123 in a patient in need thereof comprises administering to the patient an effective amount of a pharmaceutical composition comprising a recombinant polypeptide comprising a CD123 binding domain and a CD3 binding domain (e.g., TRI130 or TRI129) at any of the doses or regimens described herein. In some embodiments, a method for treating a disorder characterized by overexpression of CD123 in a patient in need thereof comprises administering to the patient an effective amount of a pharmaceutical composition comprising a recombinant polypeptide comprising a CD123 binding domain and a CD3 binding domain (e.g., TRI130 or TRI129); wherein the administration of the pharmaceutical composition induces reduced cytokine levels in the subject as compared to administration of (a) a dual affinity re-targeting antibody comprising the CD123 binding domain and the CD3 binding domain of the recombinant polypeptide; or (b) a bispecific T-cell engager molecule comprising the CD123 binding domain and the CD3 binding domain of the recombinant polypeptide. In some embodiments, the disorder is cancer, such as AML or MDS. In some embodiments, the subject was previously treated with a different CD123-binding molecule, and wherein the subject experienced an adverse event after the previous treatment. In some embodiments, the adverse event was excessive cytokine release. In some embodiments, the cytokine levels were levels of IFN-γ, TNF-α, IL-6, IL-2, IL-8, IL-10, IL-17, GM-CSF, IL-4, IL-12, IL-13 or IL-13, or any combination thereof. In some embodiments, the cytokine levels were levels of IFN-γ, IL-2, TNF-α and IL-10. In some embodiments, cytokine levels are measured in an in vitro activated T cell assay.

Pharmaceutical compositions comprising the proteins and polypeptides described herein can be supplied as a kit comprising a container that comprises the pharmaceutical composition as described herein. A pharmaceutical composition can be provided, for example, in the form of an injectable solution for single or multiple doses, or as a sterile powder that will be reconstituted before injection. Such a kit can further comprise written information on indications and usage of the pharmaceutical composition. In some embodiments, the kit comprises the pharmaceutical composition and an IV stabilizing solution (0.1 M succinate buffer, and 0.08% weight/volume polysorbate 80, at pH 6.0 or similar solution that is designed to prevent or reduce the likelihood that the multispecific polypeptides will adhere to plastic tubing and bags).

### Combination Therapies

Also provided herein are combination therapies. As used herein, the term “combination therapy” refers to administration of a first therapeutic agent and a second therapeutic agent concurrently or sequentially, in order to achieve a therapeutic benefit. For example, in some embodiments, a combination therapy comprises administration of a multispecific protein comprising a CD123 binding domain and a CD3 binding domain and a second anti-cancer agent. The multispecific protein may be referred to as “a first anti-cancer agent”. The multispecific protein and the second anti-cancer agent may be administered concurrently or sequentially, and may be administered in a single composition or in separate compositions.

In some embodiments, a method for treating a cancer comprises administering to a subject in need thereof: i) a multispecific protein comprising a CD123 binding domain and a CD3 binding domain; and ii) a second anti-cancer agent. In some embodiments, the method for treating cancer comprises administering a third, fourth, fifth, sixth, seventh, or eighth anti-cancer agent.

An anti-cancer agent may be any drug that is effective in the treatment of malignant, or cancerous, disease. There are several major classes of anti-cancer agents, including monoclonal antibodies, alkylating agents, antimetabolites, and hormones. In some embodiments, the anti-cancer agent is a chemotherapeutic drug. For example, the chemotherapeutic drug may be venetoclax, azacitidine, decitabine, daunorubicin, cytarabine, idarubicin, mitoxantrone, or etoposide. In some embodiments, a combination of one or more chemotherapeutic drugs is administered to the subject.

Venetoclax is a chemotherapy drug that is typically used to treat chronic lymphocytic leukemia, small lymphocytic lymphoma, or acute myeloid leukemia. Venetoclax is a highly selective BCL-2 inhibitor. It blocks the anti-apoptotic activity of BCL-2, thereby promoting cell death. In some embodiments, venetoclax may be administered at a dose of about 100 mg to about 400 mg per day, such as about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, or any range therebetween.

Azacitidine is a chemical analog of cytidine, a nucleoside in DNA and RNA. It is thought to have antineoplastic activity via two mechanisms—at low doses by inhibiting DNA methyltransferase (causing hypermethylation of DNA) and at high doses, by its direct cytotoxicity to abnormal hematopoietic cells in the bone marrow through its incorporation into DNA and RNA, resulting in cell death. Azacitidine and its deoxy derivative decitabine (also called 5-aza-2′-deoxycytidine) are often used in the treatment of myelodysplastic syndrome. In some embodiments, azacytidine is administered at a dose of about 50 to about 100 mg/m²/day, such as about 50, about 55, about 60, about 65, about 70, about 75, about 80, about 85, about 90, about 95, or about 100 mg/m²/day. In some embodiments, azacytidine is administered at a dose of about 75 mg/m²/day.

Decitabine is a cytidine analog that acts as a nucleotide synthesis inhibitor. It incorporates into DNA strands upon replication, and then when DNA methyltransferases (DNMTs) such as DNMT1, are engaged to bind the DNA and to replicate the methylation to the daughter strand, DNMTs are bound to decitabine irreversibly and cannot disengage. It is typically used to treat myelodysplastic syndrome and acute myeloid leukemia. In some embodiments, decitabine is administered at a dose of about 10 to about 30 mg/m²/day, such as about 10, about 15, about 20, about 25, or about 30 mg/m²/day. In some embodiments, decitabine is administered at a dose of about 20 mg/m²/day.

Daunorubicin, also known as daunomycin, is a chemotherapy drug used to treat acute myeloid leukemia, acute lymphoblastic leukemia, chronic myelogenous leukemia, and Kaposi's sarcoma. It is typically administered intravenously. Daunorubicine intercalates in DNA and inhibits macromolecular biosynthesis, blocking the function of topoisomerase II. Daunorubicin also stabilizes the topoisomerase II complex after it has broken the DNA chain for replication, preventing the DNA double helix from being resealed and thereby stopping the process of replication. In some embodiments, daunorubicin is administered at a dose of about 30-90 mg/m², such as about 30, about 35, about 40, about 45, about 50, about 55, about 60, about 65, about 70 about 75, about 80, about 85, or about 90 mg/m². In some embodiments, daunorubicin is administered at a dose of about 60 mg/m².

Cytarabine, also known as cytosine arabinoside (Ara-C) is a chemotherapy drug that is typically used to treat various forms of leukemia and lymphoma, including acute myeloid leukemia, acute lymphocytic leukemia, chronic myelogenous leukemia, and non-Hodgkin's lymphoma. It may be administered intravenously, subcutaneously, or into the cerebrospinal fluid. Cytarabine is an antimetabolite; it works by blocking the function of DNA polymerase. In some embodiments, cytarabine may be administered at a dose of about 2 to about 3 g/m² of body surface area (high dose cytarabine, or HIDAC). In some embodiments, cytarabine may be administered at a dose of about 1 g/m² of body surface area (intermediate dose cytarabine, or IDAC). In some embodiments, cytarabine may be administered at a dose of about 1.5 g/m² of body surface area. In some embodiments, cytarabine may be administered at a dose of about 100 mg to about 400 mg/m² of body surface area.

Idarubicin, also called 4-demethoxydanorubicin, is a chemotherapy drug that inserts itself into DNA and prevents DNA unwinding by interfering with the enzyme topoisomerase II. It is an analog of danorubicin, but the absence of a methoxy group increases its fat solubility and cellular uptake. It also induces histone eviction from chromatin. It is used for treatment of acute lymphoblastic leukemia and chronic myelogenous leukemia. In some embodiments, idarubicin is administered at a dose of about 1 to about 20 mg/m² daily, such as about 1, about 2, about 3, about 4, about 5, about 6, about 7, about 8, about 9, about 10, about 11, about 12, about 13, about 14, about 15, about 16, about 17, about 18, about 19 or about 20 mg/m² daily. In some embodiments, idarubicin is administered at a dose of about 12 mg/m² daily.

Mitoxantrone is an anthracenedione antibiotic with antineoplastic activity. Mitoxantrone intercalates into and crosslinks DNA, thereby disrupting DNA and RNA replication. This agent also binds to topoisomerase II, resulting in DNA strand breaks and inhibition of DNA repair. It is used in the treatment of acute leukemia, lymphoma, and prostate and breast cancer, but also for late stage, severe multiple sclerosis. In some embodiments, mitoxantrone is administered at a dose of about 1 to about 20 mg/m²/day, such as about 1, about 2, about 3, about 4, about 5, about 6, about 7, about 8, about 9, about 10, about 11, about 12, about 13, about 14, about 15, about 16, about 17, about 18, about 19, or about 20 mg/m²/day. In some embodiments, mitoxantrone is administered at a dose of about 6 mg/m²/day.

Etoposide is used for treatment of testicular cancer, lung cancer, lymphoma, leukemia, neuroblastoma, and ovarian cancer, as well as hemophagocytic lymphohistiocytosis. It may be administered orally, or intravenously. Etoposide forms a ternary complex with DNA and the topoisomerase II enzyme, prevents re-ligation of the DNA strands, and causes DNA strand breakage. This leads to errors in DNA synthesis and apoptosis of the cancer cells. In some embodiments, etoposide is administered at a dose of about 10 to about 100 mg/m²/day, such as about 10, about 20, about 30, about 40, about 50, about 60, about 70, about 80, about 90, or about 100 mg/m²/day.

In some embodiments, a method for treating a cancer comprises administering to a subject in need thereof: a multispecific protein as described herein and more than one additional anti-cancer agents. The more than one additional anti-cancer agents may be individually selected from venetoclax, azacitidine, decitabine, daunorubicin, cytarabine, idarubicin, mitoxantrone, and etoposide. The cancer may be, for example, a carcinoma or sarcoma. In some embodiments, the cancer is melanoma, kidney cancer, pancreatic cancer, lung cancer, intestinal cancer, prostate cancer, breast cancer, liver cancer, brain cancer, colon cancer, ovarian cancer, or hematological cancer. In some embodiments, the cancer is acute myeloid leukemia (AML), myelodysplastic syndrome (MDS), hairy cell leukemia (HCL), blastic plasmacytoid dendritic cell neoplasm, B-cell acute lymphoblastic leukemia (ALL), or chronic myeloid leukemia (CML). In some embodiments, the cancer is acute myeloid leukemia (AML). In some embodiments, the cancer is myelodysplastic syndrome (MDS).

In some embodiments, a method for treating a cancer comprises administering to a subject in need thereof: a multispecific protein as described herein, and mitoxantrone, etoposide, and cytarabine. This combination of chemotherapy drugs is also referred to herein as “MEC.” In some embodiments, mitoxantrone, etoposide, and cytarabine are administered to a subject, wherein mitoxantrone is administered at a dose of about 6 mg/m²/day, etoposide is administered at a dose of about 80 mg/m²/day, and cytarabine is administered at a dose of about 1 g/m²/day. The mitoxantrone, etoposide, and cytarabine may be administered at the same time, or may be administered sequentially.

In some embodiments, a method for treating a cancer comprises administering to a subject in need thereof: a multispecific protein as described herein, azacytidine and venetoclax. In some embodiments, the azacitidine is administered at a dose of about 75 mg/m²/day and the venetoclax is administered at a dose of from about 100 to about 400 mg/day. The azacytidine and venetoclax may be administered at the same time or may be administered sequentially.

In some embodiments, a method for treating a cancer comprises administering to a subject in need thereof: a multispecific protein as described herein, decidabine and venetoclax. In some embodiments, the decitabine is administered at a dose of about 20 mg/m²/day and the venetoclax is administered at a dose of from about 100 to about 400 mg/day. The decidabine and venetoclax may be administered at the same time or may be administered sequentially.

In some embodiments, a method for treating a cancer comprises administering to a subject in need thereof: a multispecific protein as described herein, daunorubicin and cytarabine. In some embodiments, the daunorubicin is administered at a dose of about 30-90 mg/m² and the cytarabine is administered at a dose of from about 100 to about 200 mg/m². The daunorubicin and cytarabine may be administered at the same time or may be administered sequentially.

In some embodiments, a method for treating a cancer comprises administering to a subject in need thereof: a multispecific protein as described herein, idarubicin and cytarabine. In some embodiments, the idarubicin is administered at a dose of about 12 mg/m², and cytarabine is administered at a dose of from about 100 to about 200 mg/m². The idarubicin and cytarabine may be administered at the same time or may be administered sequentially.

In some embodiments, a method for treating a cancer comprises administering to a subject in need thereof: i) a multispecific protein comprising a CD123 binding domain and a CD3 binding domain; and ii) a second anti-cancer agent, wherein the multispecific protein is administered by intravenous infusion and the second anti-cancer agent is administered by either intravenous infusion or orally. In some embodiments, the multispecific protein is administered the subject by IV infusion at a dose of 0.3, 1, 3, 6, 9, 12, 18, 20, 24, 30, 36, 48, 50, 60, 75, or 100 μg. In some embodiments, the multispecific protein is administered once or twice per week.

In some embodiments, the multispecific protein is administered to the subject by IV infusion during a first 28-day cycle; wherein 6 μg of the multispecific protein is administered on day 8; 12 μg of the multispecific protein is administered on day 15; and 18 μg of the multispecific protein is administered on day 22 of the first 28-day cycle. In some embodiments, the multispecific protein is administered to the subject by IV during at least one additional 28-day cycle following the first 28-day cycle, wherein 18 μg of the multispecific protein is administered on days 1, 8, 15, and 22 of the at least one additional 28-day cycle. In some embodiments, cytarabine is administered intravenously on days 1-5 of the first 28-day cycle, and days 1-5 of at least one additional 28-day cycle. In some embodiments, the dose of cytarabine is about 1 g/m². In some embodiments, mitoxantrone, etoposide, and cytarabine are administered intravenously on days 1-6 of the first 28-day cycle, and at least one additional 28-day cycle. In some embodiments, the dose of mitoxantrone is about 6 mg/m²/day, the dose of etoposide is about 80 mg/m²/day, and the dose of cytarabine is about 1 g/m²/day.

In some embodiments, the multispecific protein is administered to the subject by IV infusion during a first 28-day cycle; wherein 6 μg of the multispecific protein is administered on day 15; and 12 μg of the multispecific protein is administered on day 22 of the first 28-day cycle. In some embodiments, the multispecific protein is administered to the subject by IV infusion during at least one additional 28-day cycle following the first 28-day cycle, wherein 18 μg of the multispecific protein is administered on days 1, 8, 15, and 22 of the at least one additional 28-day cycle. In some embodiments, venetoclax is administered orally on days 1-21 of the first 28-day cycle, and days 1-21 of at least one additional 28-day cycle. In some embodiments, the dose of venetoclax is from about 100 to about 400 mg/day. In some embodiments, azacytidine is administered intravenously on days 1-7 of the first 28-day cycle, and at least one additional 28-day cycle. In some embodiments, the dose of azacytidine is about 75 mg/m².

In some embodiments, the multispecific protein is administered to the subject by IV infusion during a first 28-day cycle, wherein 6 μg of the multispecific protein is administered on day 1; 8 μg of the multispecific protein is administered on day 12; 18 μg of the multispecific protein is administered on day 15; and 18 μg of the multispecific protein is administered on day 22 of the first 28-day cycle. In some embodiments, the multispecific protein is administered to the subject by IV infusion during at least one additional 28-day cycle following the first 28-day cycle, wherein 18 μg of the multispecific protein is administered on days 1, 8, 15, and 22. In some embodiments, cytarabine is administered by intravenous infusion on days 1-7 of the first 28-day cycle, and days 1-7 of at least one additional 28-day cycle. In some embodiments, the dose of cytarabine is from about 100 to about 200 mg/m². In some embodiments, idarubicin is administered by intravenous infusion on days 1-3 of the first 28-day cycle, and days 1-3 of at least one additional 28-day cycle. In some embodiments, the dose of idarubicin is about 12 mg/m².

In some embodiments, the multispecific protein is administered to the subject by IV infusion during a first 28-day cycle, wherein 6 μg of the multispecific protein is administered on day 1; 8 μg of the multispecific protein is administered on day 12; 18 μg of the multispecific protein is administered on day 15; and 18 μg of the multispecific protein is administered on day 22 of the first 28-day cycle. In some embodiments, the multispecific protein is administered to the subject by IV infusion during at least one additional 28-day cycle following the first 28-day cycle, wherein 18 μg of the multispecific protein is administered on days 1, 8, 15, and 22 of the at least one additional 28-day cycle. In some embodiments, azacytidine is administered orally on days 1-14 of the first 28-day cycle, and at least one additional 28-day cycle. In some embodiments, the dose of azacytidine is about 300 mg/day.

### Monotherapies

Also provided herein are compositions and methods for use of a multispecific protein comprising a CD123 binding domain and a CD3 binding domain as a monotherapy for treating cancer.

In some embodiments, the multispecific protein is administered to the subject as a monotherapy, i.e., without any second anti-cancer agent. In some embodiments, the multispecific protein is administered once per week, for example at a dose of 1, 3, 9, 12, 18, 24, 36, 48, or 60 μg. In some embodiments, the weekly target dose level for the multispecific protein is 1, 3, 9, 12, 18, 24, 36, 48, or 60 μg. In some embodiments, the multispecific protein is administered once per week at a dose of 0.1, 0.2, 0.3, 0.4, 0.5, 0.6, 0.7, or 0.8 μg/kg.

In some embodiments, the multispecific protein is administered twice weekly during a first 28-day cycle and at least one additional 28-day cycle. For example, in some embodiments, the multispecific protein is administered to the subject by IV infusion during a first 28-day cycle, wherein 6 μg of the multispecific protein is administered on day 1; 6 μg of the multispecific protein is administered on day 4; 12 μg of the multispecific protein is administered on day 8; 12 μg of the multispecific protein is administered on day 11; 18 μg of the multispecific protein is administered on day 15; 18 μg of the multispecific protein is administered on day 18; 18 μg of the multispecific protein is administered on day 22; and 18 μg of the multispecific protein is administered on day 11; of the first 28-day cycle. In some embodiments, the multispecific protein is administered to the subject as a monotherapy for at least one additional 28-day cycle, such as 1, 2, 3, 4, 5, 6, 7, 8, or more additional 28-day cycles.

In some embodiments, a method for treating a cancer comprises administering to a subject in need thereof a multispecific protein comprising a CD123 binding domain and a CD3 binding domain (i.e., as a monotherapy). In some embodiments, the multispecific protein comprises: a dimer of two identical polypeptides, wherein each polypeptide comprises, in order from amino-terminus to carboxyl-terminus, or in order from carboxyl-terminus to amino-terminus: (i) a CD123 binding domain, (ii) a hinge region, (iii) an immunoglobulin constant region, and (iv) a CD3 binding domain. In some embodiments, the polypeptide comprises, from N-terminus to C-terminus, the CD123 binding domain, the hinge region, the immunoglobulin constant region, and the CD3 binding domain. In some embodiments, the CD123 and the CD3 binding domains comprises: (i) an immunoglobulin heavy chain variable region (V_{H}) comprising HCDR1, HCDR2, and HCDR3; and (ii) an immunoglobulin light chain variable region (V_{L}) comprising LCDR1, LCDR2, and LCDR3. In some embodiments, the CD123 binding domain is a scFv comprising: a HCDR1 that comprises SEQ ID NO: 10, a HCDR2 that comprises SEQ ID NO: 11, and a HDCR3 that comprises SEQ ID NO: 12; and a LCDR1 that comprises SEQ ID NO: 13, a LCDR2 that comprises SEQ ID NO: 14, and a LCDR3 that comprises SEQ ID NO: 15. In some embodiments, the CD123 binding domain is a scFv comprising: a V_{H} comprising a sequence at least 90%, at least 95%, or 100% identical to SEQ ID NO: 136, and a V_{L} comprising a sequence at least 90%, at least 95%, or 100% identical to SEQ ID NO: 134. In some embodiments, the CD123 binding domain is a scFv, and wherein the scFv comprises a sequence at least 90%, at least 95%, or 100% identical to SEQ ID NO: 27. In some embodiments, the CD3 binding domain is a scFv comprising: a HCDR1 that comprises SEQ ID NO: 19, a HCDR2 that comprises SEQ ID NO: 20, and a HDCR3 that comprises SEQ ID NO: 21; and a LCDR1 that comprises SEQ ID NO: 22, a LCDR2 that comprises SEQ ID NO: 23, and a LCDR3 that comprises SEQ ID NO: 24. In some embodiments, the CD3 binding domain is a scFv that comprises: a V_{H} comprising a sequence at least 90%, at least 95%, or 100% identical to SEQ ID NO: 383 or 387, and a V_{L} comprising a sequence at least 90%, at least 95%, or 100% identical to SEQ ID NO: 384. In some embodiments, the CD3 binding domain is a scFv that comprises a sequence at least 90%, at least 95%, or 100% identical to SEQ ID NO: 27. In some embodiments, each polypeptide comprises a sequence at least 90%, at least 95%, or 100% identical to SEQ ID NO: 31. In some embodiments, the multispecific protein is administered to the subject by IV infusion.

In some embodiments, the multispecific protein is administered the subject by IV infusion at a dose of 0.1, 0.2, 0.3, 0.4, 0.5, 0.6, 0.7, or 0.8 μg/kg. In some embodiments, the multispecific protein is administered the subject by IV infusion at a dose of 0.3, 1, 3, 6, 9, 12, 18, 20, 24, 30, 36, 48, 50, 60, 75, or 100 μg. In some embodiments, the multispecific protein is administered once per week.

In some embodiments, a multispecific protein is administered to the subject by IV infusion during a first 28-day cycle; wherein 6 μg of the multispecific protein is administered on day 8; 12 μg of the multispecific protein is administered on day 15; and 18 μg of the multispecific protein is administered on day 22 of the first 28-day cycle. In some embodiments, the multispecific protein is administered to the subject by IV infusion during at least one additional 28-day cycle following the first 28-day cycle, wherein 18 μg of the multispecific protein is administered on days 1, 8, 15, and 22 of the at least one additional 28-day cycle.

In some embodiments, the multispecific protein is administered to the subject by IV infusion during a first 28-day cycle; wherein 6 μg of the multispecific protein is administered on day 15; and 12 μg of the multispecific protein is administered on day 22 of the first 28-day cycle. In some embodiments, the multispecific protein is administered to the subject by IV infusion during at least one additional 28-day cycle following the first 28-day cycle, wherein 18 μg of the multispecific protein is administered on days 1, 8, 15, and 22 of the at least one additional 28-day cycle.

In some embodiments, the multispecific protein is administered to the subject by IV infusion during a first 28-day cycle, wherein 6 μg of the multispecific protein is administered on day 1; 8 μg of the multispecific protein is administered on day 12; 18 μg of the multispecific protein is administered on day 15; and 18 μg of the multispecific protein is administered on day 22 of the first 28-day cycle. In some embodiments, the multispecific protein is administered to the subject by IV infusion during at least one additional 28-day cycle following the first 28-day cycle, wherein 18 μg of the multispecific protein is administered on days 1, 8, 15, and 22.

In some embodiments, the multispecific protein is administered to the subject by IV infusion during a first 28-day cycle, wherein 6 μg of the multispecific protein is administered on day 1; 8 μg of the multispecific protein is administered on day 12; 18 μg of the multispecific protein is administered on day 15; and 18 μg of the multispecific protein is administered on day 22 of the first 28-day cycle. In some embodiments, the multispecific protein is administered to the subject by IV infusion during at least one additional 28-day cycle following the first 28-day cycle, wherein 18 μg of the multispecific protein is administered on days 1, 8, 15, and 22 of the at least one additional 28-day cycle.

In some embodiments, the cancer is a carcinoma or sarcoma. In some embodiments, the cancer is melanoma, kidney cancer, pancreatic cancer, lung cancer, intestinal cancer, prostate cancer, breast cancer, liver cancer, brain cancer, colon cancer, ovarian cancer, or hematological cancer. In some embodiments, the cancer is acute myeloid leukemia (AML), myelodysplastic syndrome (MDS), hairy cell leukemia (HCL), blastic plasmacytoid dendritic cell neoplasm, B-cell acute lymphoblastic leukemia (ALL), or chronic myeloid leukemia (CML). In some embodiments, the cancer is acute myeloid leukemia (AML). In some embodiments, the cancer is myelodysplastic syndrome (MDS).

### NUMBERED EMBODIMENTS

Notwithstanding the appended claims, the disclosure sets forth the following numbered embodiments:

1. A method for treating a cancer, the method comprising administering to a subject in need thereof: i) a multispecific protein comprising a CD123 binding domain and a CD3 binding domain; and ii) a second anti-cancer agent.

2. The method of embodiment 1, wherein the method further comprises administering to the subject a third anti-cancer agent.

3. The method of embodiment 1 or 2, wherein the second anti-cancer agent is a chemotherapeutic drug.

4. The method of embodiment 3, wherein the chemotherapeutic drug is venetoclax, azacitidine, decitabine, daunorubicin, cytarabine, idarubicin, mitoxantrone, or etoposide.

5. The method of embodiment 1, wherein the second anti-cancer agent is cytarabine.

6. The method of embodiment 5, wherein the cytarabine is administered at a dose of about 1 g/m².

7. The method of embodiment 2, wherein the second anti-cancer agent is mitoxantrone, and the third anti-cancer agent is etoposide, and wherein the method further comprises administering a fourth anti-cancer agent that is cytarabine.

8. The method of embodiment 7, wherein the mitoxantrone is administered at a dose of about 6 mg/m²/day, the etoposide is administered at a dose of about 80 mg/m²/day, and the cytarabine is administered at a dose of about 1 g/m²/day.

9. The method of embodiment 1, wherein the second anti-cancer agent is venetoclax.

10. The method of embodiment 9, wherein the venetoclax is administered at a dose of from about 100 to about 400 mg per day.

11. The method of embodiment 1, wherein the second anti-cancer agent is azacytidine.

12. The method of embodiment 11, wherein the azacytidine is administered at a dose of about 75 mg/m²/day.

13. The method of embodiment 2, wherein the second anti-cancer agent is azacitidine and the third anti-cancer agent is venetoclax.

14. The method of embodiment 13, wherein the azacitidine is administered at a dose of about 75 mg/m²/day and the venetoclax is administered at a dose of from about 100 to about 400 mg/day.

15. The method of embodiment 1, wherein the second anti-cancer agent is decitabine.

16. The method of embodiment 15, wherein the decitabine is administered at a dose of about 20 mg/m²/day.

17. The method of embodiment 2, wherein the second anti-cancer agent is decitabine and the third anti-cancer agent is venetoclax.

18. The method of embodiment 17, wherein the decitabine is administered at a dose of about 20 mg/m²/day and the venetoclax is administered at a dose of from about 100 to about 400 mg/day.

19. The method of embodiment 2, wherein the second anti-cancer agent is daunorubicin, and the third anti-cancer agent is cytarabine.

20. The method of embodiment 19, wherein the daunorubicin is administered at a dose of about 30-90 mg/m² and the cytarabine is administered at a dose of from about 100 to about 200 mg/m².

21. The method of embodiment 1, wherein the anti-cancer agent is idarubicin.

22. The method of embodiment 21, wherein the idarubicin is administered at a dose of about 12 mg/m².

23. The method of embodiment 2, wherein the second anti-cancer agent is idarubicin and the third anti-cancer agent is cytarabine.

24. The method of embodiment 23, wherein the idarubicin is administered at a dose of about 12 mg/m², and cytarabine is administered at a dose of from about 100 to about 200 mg/m².

25. The method of embodiment 1, wherein the second anti-cancer agent is azacitidine.

26. The method of embodiment 5, wherein the azacitidine is administered at a dose of about 75 mg/m²/day.

27. The method of any one of embodiments 1-26, wherein the multispecific protein comprises: a dimer of two identical polypeptides, wherein each polypeptide comprises, in order from amino-terminus to carboxyl-terminus, or in order from carboxyl-terminus to amino-terminus: (i) a CD123 binding domain, (ii) a hinge region, (iii) an immunoglobulin constant region, and (iv) a CD3 binding domain.

28. The method of embodiment 27, wherein the polypeptide comprises, from N-terminus to C-terminus, the CD123 binding domain, the hinge region, the immunoglobulin constant region, and the CD3 binding domain.

29. The method of embodiment 28, wherein at least one of the CD123 and the CD3 binding domains comprises: (i) an immunoglobulin heavy chain variable region (VH) comprising HCDR1, HCDR2, and HCDR3; and (ii) an immunoglobulin light chain variable region (VL) comprising LCDR1, LCDR2, and LCDR3.

30. The method of embodiment 29, wherein the CD123 binding domain is a scFv comprising: a HCDR1 that comprises SEQ ID NO: 10, a HCDR2 that comprises SEQ ID NO: 11, and a HDCR3 that comprises SEQ ID NO: 12; and a LCDR1 that comprises SEQ ID NO: 13, a LCDR2 that comprises SEQ ID NO: 14, and a LCDR3 that comprises SEQ ID NO: 15.

31. The method of embodiment 29, wherein the CD123 binding domain is a scFv comprising: a VH comprising a sequence at least 90%, at least 95%, or 100% identical to SEQ ID NO: 136, and a VL comprising a sequence at least 90%, at least 95%, or 100% identical to SEQ ID NO: 134.

32. The method of embodiment 29, wherein the CD123 binding domain is a scFv, and wherein the scFv comprises a sequence at least 90%, at least 95%, or 100% identical to SEQ ID NO: 27.

33. The method of any one of embodiments 29-32, wherein the CD3 binding domain is a scFv comprising: a HCDR1 that comprises SEQ ID NO: 19, a HCDR2 that comprises SEQ ID NO: 20, and a HDCR3 that comprises SEQ ID NO: 21; and a LCDR1 that comprises SEQ ID NO: 22, a LCDR2 that comprises SEQ ID NO: 23, and a LCDR3 that comprises SEQ ID NO: 24.

34. The method of any one of embodiments 29-32, wherein the CD3 binding domain is a scFv that comprises: a VH comprising a sequence at least 90%, at least 95%, or 100% identical to SEQ ID NO: 383 or 387, and a VL comprising a sequence at least 90%, at least 95%, or 100% identical to SEQ ID NO: 384.

35. The method of any one of embodiments 29-32, wherein the CD3 binding domain is a scFv that comprises a sequence at least 90%, at least 95%, or 100% identical to SEQ ID NO: 27.

36. The method of embodiment 29, wherein each polypeptide comprises a sequence at least 90%, at least 95%, or 100% identical to SEQ ID NO: 31.

37. The method of any one of embodiments 1-36, wherein the multispecific protein is administered to the subject by IV infusion.

38. The method of any one of embodiments 1-37, wherein the second anti-cancer agent is administered to the subject orally or by IV infusion.

39. The method of any one of embodiments 1-38, wherein the multispecific protein is administered the subject by IV infusion at a dose of 0.3, 1, 3, 6, 9, 12, 18, 20, 24, 30, 36, 48, 50, 60, 75, or 100 μg.

40. The method of any one of embodiments 1-39, wherein the multispecific protein is administered once per week.

41. The method of embodiment 40, wherein the multispecific protein is administered to the subject by IV infusion during a first 28-day cycle; wherein 6 μg of the multispecific protein is administered on day 8; 12 μg of the multispecific protein is administered on day 15; and 18 μg of the multispecific protein is administered on day 22 of the first 28-day cycle.

42. The method of embodiment 41, wherein the multispecific protein is administered to the subject by IV infusion during at least one additional 28-day cycle following the first 28-day cycle, wherein 18 μg of the multispecific protein is administered on days 1, 8, 15, and 22 of the at least one additional 28-day cycle.

43. The method of embodiment 42, wherein cytarabine is administered intravenously on days 1-5 of the first 28-day cycle, and days 1-5 of at least one additional 28-day cycle.

44. The method of embodiment 43, wherein the dose of cytarabine is about 1 g/m².

45. The method of embodiment 42, wherein mitoxantrone, etoposide, and cytarabine are administered intravenously on days 1-6 of the first 28-day cycle, and at least one additional 28-day cycle.

46. The method of embodiment 45, wherein the dose of mitoxantrone is about 6 mg/m²/day, the dose of etoposide is about 80 mg/m²/day, and the dose of cytarabine is about 1 g/m²/day.

47. The method of embodiment 40, wherein the multispecific protein is administered to the subject by IV infusion during a first 28-day cycle; wherein 6 μg of the multispecific protein is administered on day 15; and 12 μg of the multispecific protein is administered on day 22 of the first 28-day cycle.

48. The method of embodiment 47, wherein the multispecific protein is administered to the subject by IV infusion during at least one additional 28-day cycle following the first 28-day cycle, wherein 18 μg of the multispecific protein is administered on days 1, 8, 15, and 22 of the at least one additional 28-day cycle.

49. The method of embodiment 48, wherein venetoclax is administered orally on days 1-21 of the first 28-day cycle, and days 1-21 of at least one additional 28-day cycle.

50. The method of embodiment 49, wherein the dose of venetoclax is from about 100 to about 400 mg/day.

51. The method of any one of embodiments 48-50, wherein azacytidine is administered intravenously on days 1-7 of the first 28-day cycle, and at least one additional 28-day cycle.

52. The method of embodiment 51, wherein the dose of azacytidine is about 75 mg/m².

53. The method of embodiment 40, wherein the multispecific protein is administered to the subject by IV infusion during a first 28 day cycle, wherein 6 μg of the multispecific protein is administered on day 1; 8 μg of the multispecific protein is administered on day 12; 18 μg of the multispecific protein is administered on day 15; and 18 μg of the multispecific protein is administered on day 22 of the first 28-day cycle.

54. The method of embodiment 53, wherein the multispecific protein is administered to the subject by IV infusion during at least one additional 28-day cycle following the first 28-day cycle, wherein 18 μg of the multispecific protein is administered on days 1, 8, 15, and 22.

55. The method of embodiment 54, wherein cytarabine is administered by intravenous infusion on days 1-7 of the first 28-day cycle, and days 1-7 of at least one additional 28-day cycle.

56. The method of embodiment 55, wherein the dose of cytarabine is from about 100 to about 200 mg/m².

57. The method of any one of embodiments 53-56, wherein idarubicin is administered by intravenous infusion on days 1-3 of the first 28-day cycle, and days 1-3 of at least one additional 28-day cycle.

58. The method of embodiment 57, wherein the dose of idarubicin is about 12 mg/m².

59. The method of embodiment 40, wherein the multispecific protein is administered to the subject by IV infusion during a first 28-day cycle, wherein 6 μg of the multispecific protein is administered on day 1; 8 μg of the multispecific protein is administered on day 12; 18 μg of the multispecific protein is administered on day 15; and 18 μg of the multispecific protein is administered on day 22 of the first 28-day cycle.

60. The method of embodiment 59, wherein the multispecific protein is administered to the subject by IV infusion during at least one additional 28-day cycle following the first 28-day cycle, wherein 18 μg of the multispecific protein is administered on days 1, 8, 15, and 22 of the at least one additional 28-day cycle.

61. The method of embodiment 60, wherein azacytidine is administered orally on days 1-14 of the first 28-day cycle, and at least one additional 28-day cycle.

62. The method of embodiment 61, wherein the dose of azacytidine is about 300 mg/day.

63. The method of any one of embodiments 1-62, wherein the cancer is a carcinoma or sarcoma.

64. The method of any one of embodiments 1-62, wherein the cancer is melanoma, kidney cancer, pancreatic cancer, lung cancer, intestinal cancer, prostate cancer, breast cancer, liver cancer, brain cancer, colon cancer, ovarian cancer, or hematological cancer.

65. The method of any one of embodiments 1-62, wherein the cancer is acute myeloid leukemia (AML), myelodysplastic syndrome (MDS), hairy cell leukemia (HCL), blastic plasmacytoid dendritic cell neoplasm, B-cell acute lymphoblastic leukemia (ALL), or chronic myeloid leukemia (CML).

66. The method of any one of embodiments 1-62, wherein the cancer is acute myeloid leukemia (AML).

67. The method of any one of embodiments 1-62, wherein the cancer is myelodysplastic syndrome (MDS).

68. A method for treating a cancer, the method comprising administering to a subject in need thereof a multispecific protein comprising a CD123 binding domain and a CD3 binding domain.

69. The method of embodiment 68, wherein the multispecific protein comprises: a dimer of two identical polypeptides, wherein each polypeptide comprises, in order from amino-terminus to carboxyl-terminus, or in order from carboxyl-terminus to amino-terminus: (i) a CD123 binding domain, (ii) a hinge region, (iii) an immunoglobulin constant region, and (iv) a CD3 binding domain.

70. The method of embodiment 69, wherein the polypeptide comprises, from N-terminus to C-terminus, the CD123 binding domain, the hinge region, the immunoglobulin constant region, and the CD3 binding domain.

71. The method of embodiment 69, wherein at least one of the CD123 and the CD3 binding domains comprises: (i) an immunoglobulin heavy chain variable region (VH) comprising HCDR1, HCDR2, and HCDR3; and (ii) an immunoglobulin light chain variable region (VL) comprising LCDR1, LCDR2, and LCDR3.

72. The method of embodiment 69, wherein the CD123 binding domain is a scFv comprising: a HCDR1 that comprises SEQ ID NO: 10, a HCDR2 that comprises SEQ ID NO: 11, and a HDCR3 that comprises SEQ ID NO: 12; and a LCDR1 that comprises SEQ ID NO: 13, a LCDR2 that comprises SEQ ID NO: 14, and a LCDR3 that comprises SEQ ID NO: 15.

73. The method of embodiment 69, wherein the CD123 binding domain is a scFv comprising: a VH comprising a sequence at least 90%, at least 95%, or 100% identical to SEQ ID NO: 136, and a VL comprising a sequence at least 90%, at least 95%, or 100% identical to SEQ ID NO: 134.

74. The method of embodiment 69, wherein the CD123 binding domain is a scFv, and wherein the scFv comprises a sequence at least 90%, at least 95%, or 100% identical to SEQ ID NO: 27.

75. The method of any one of embodiments 69-74, wherein the CD3 binding domain is a scFv comprising: a HCDR1 that comprises SEQ ID NO: 19, a HCDR2 that comprises SEQ ID NO: 20, and a HDCR3 that comprises SEQ ID NO: 21; and a LCDR1 that comprises SEQ ID NO: 22, a LCDR2 that comprises SEQ ID NO: 23, and a LCDR3 that comprises SEQ ID NO: 24.

76. The method of any one of embodiments 69-74, wherein the CD3 binding domain is a scFv that comprises: a VH comprising a sequence at least 90%, at least 95%, or 100% identical to SEQ ID NO: 383 or 387, and a VL comprising a sequence at least 90%, at least 95%, or 100% identical to SEQ ID NO: 384.

77. The method of any one of embodiments 69-74, wherein the CD3 binding domain is a scFv that comprises a sequence at least 90%, at least 95%, or 100% identical to SEQ ID NO: 27.

78. The method of embodiment 69, wherein each polypeptide comprises a sequence at least 90%, at least 95%, or 100% identical to SEQ ID NO: 31.

79. The method of any one of embodiments 68-78, wherein the multispecific protein is administered to the subject by IV infusion.

80. The method of any one of embodiments 68-79, wherein the multispecific protein is administered the subject by IV infusion at a dose of 0.1, 0.2, 0.3, 0.4, 0.5, 0.6, 0.7, or 0.8 μg/kg.

81. The method of any one of embodiments 68-79, wherein the multispecific protein is administered the subject by IV infusion at a dose of 0.3, 1, 3, 6, 9, 12, 18, 20, 24, 30, 36, 48, 50, 60, 75, or 100 μg.

82. The method of any one of embodiments 68-81, wherein the multispecific protein is administered once per week.

83. The method of embodiment 82, wherein the multispecific protein is administered to the subject by IV infusion during a first 28-day cycle; wherein 6 μg of the multispecific protein is administered on day 8; 12 μg of the multispecific protein is administered on day 15; and 18 μg of the multispecific protein is administered on day 22 of the first 28-day cycle.

84. The method of embodiment 83, wherein the multispecific protein is administered to the subject by IV infusion during at least one additional 28-day cycle following the first 28-day cycle, wherein 18 μg of the multispecific protein is administered on days 1, 8, 15, and 22 of the at least one additional 28-day cycle.

85. The method of embodiment 82, wherein the multispecific protein is administered to the subject by IV infusion during a first 28-day cycle; wherein 6 μg of the multispecific protein is administered on day 15; and 12 μg of the multispecific protein is administered on day 22 of the first 28-day cycle.

86. The method of embodiment 85, wherein the multispecific protein is administered to the subject by IV infusion during at least one additional 28-day cycle following the first 28-day cycle, wherein 18 μg of the multispecific protein is administered on days 1, 8, 15, and 22 of the at least one additional 28-day cycle.

87. The method of embodiment 82, wherein the multispecific protein is administered to the subject by IV infusion during a first 28-day cycle, wherein 6 μg of the multispecific protein is administered on day 1; 8 μg of the multispecific protein is administered on day 12; 18 μg of the multispecific protein is administered on day 15; and 18 μg of the multispecific protein is administered on day 22 of the first 28-day cycle.

88. The method of embodiment 87, wherein the multispecific protein is administered to the subject by IV infusion during at least one additional 28-day cycle following the first 28-day cycle, wherein 18 μg of the multispecific protein is administered on days 1, 8, 15, and 22.

89. The method of embodiment 82, wherein the multispecific protein is administered to the subject by IV infusion during a first 28-day cycle, wherein 6 μg of the multispecific protein is administered on day 1; 8 μg of the multispecific protein is administered on day 12; 18 μg of the multispecific protein is administered on day 15; and 18 μg of the multispecific protein is administered on day 22 of the first 28-day cycle.

90. The method of embodiment 89, wherein the multispecific protein is administered to the subject by IV infusion during at least one additional 28-day cycle following the first 28-day cycle, wherein 18 μg of the multispecific protein is administered on days 1, 8, 15, and 22 of the at least one additional 28-day cycle.

91. The method of any one of embodiments 68-90, wherein the cancer is a carcinoma or sarcoma.

92. The method of any one of embodiments 68-90, wherein the cancer is melanoma, kidney cancer, pancreatic cancer, lung cancer, intestinal cancer, prostate cancer, breast cancer, liver cancer, brain cancer, colon cancer, ovarian cancer, or hematological cancer.

93. The method of any one of embodiments 68-90, wherein the cancer is acute myeloid leukemia (AML), myelodysplastic syndrome (MDS), hairy cell leukemia (HCL), blastic plasmacytoid dendritic cell neoplasm, B-cell acute lymphoblastic leukemia (ALL), or chronic myeloid leukemia (CML).

94. The method of any one of embodiments 68-90, wherein the cancer is acute myeloid leukemia (AML).

95. The method of any one of embodiments 68-90, wherein the cancer is myelodysplastic syndrome (MDS).

The disclosure will be further clarified by the following examples, which are intended to be purely exemplary of the disclosure and in no way limiting.

### EXAMPLES

The invention is further described in detail by reference to the following examples. These examples are provided for purposes of illustration only and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples, therefore, specifically point out the preferred embodiments of the present invention and are not to be construed as limiting in any way the remainder of the disclosure.

### Example 1: Determination of the No Observed Adverse Effect Level (NOAEL) and the Minimum Anticipated Biological Effect Level (MABEL) for TRI130

This example describes experiments used to determine the NOAEL and MABEL for TRI130, a multispecific protein comprising a CD123 binding domain and a CD3 binding domain. This data was used to establish human dosing cohorts for the clinical trials described in the following examples.

### The No Observed Adverse Effect Level (NOAEL)

A 28-day repeat-dose toxicology study with a 5-week recovery period was conducted in non-human primates (NHP). Animals in four study groups received weekly doses of vehicle, or 0.5, 2.5, or 10 mg/kg of TRI130. Parameters measured included safety pharmacology, laboratory evaluations, and necropsy with full histopathology. There were no clinical adverse findings, no changes in animal weights or organ weights, and no abnormal macroscopic or microscopic findings in histopathology related to TRI130. Minimal cytokines were detected after dosing and were attenuated after the second dose compared to the first dose. The expected pharmacodynamic effect of the anti-CD3 binding domain of the molecule was observed with transient redistribution of T cells. The elimination half-life was approximately 73 hours at the high dose. The no observed adverse effect level (NOAEL) was 10 mg/kg in NHP, which translates into a human equivalent dose (HED) of approximately 3.2 mg/kg.

### Minimum Anticipated Biological Effect Level (MABEL)

TRI130 mobilizes T cells to lyse tumor cells expressing the target antigen CD123, and the maximum activity occurs at very low levels of TCR occupancy (data not shown). To calculate the minimum anticipated biologic effect level (MABEL), in vitro activity assays were used in place of in vitro receptor occupancy.

The MABEL was determined using the effective concentration necessary to elicit 10% activity (EC10) (Muller et al., 2009) in a human T-cell activation in vitro assay. While the potency of TRI130 in these T-cell activation assays may vary depending on the degree of activation of T cells and the effector-to-target (E:T) cell ratio, the activation assay represents a more sensitive assay for calculation of MABEL, compared to the redirected T cell cytotoxicity (RTCC) assay. Both RTCC and T-cell activation were evaluated as possible assays to predict the MABEL for the starting clinical dose. The RTCC assay, using purified T cells at a 10:1 E:T ratio with CD123+KG1a tumor cell line had an average EC10 of 5.8 pM, assessed from 5 donors (a range of 4.4 to 6.7 pM across the donors evaluated). In contrast, the T-cell activation assay was a more sensitive assay to estimate the MABEL. To measure T-cell activation induced in vitro, T cells were isolated from peripheral blood mononuclear cells and incubated with TRI130 in the presence of CD123+ tumor cells (MOLM-13). Upregulation of CD69 and CD25 on T cells was monitored at 20 hours using multi-color flow cytometry, after gating on live CD4+ and CD8+ T cells. These assays assessed three donors, for activation of both CD4 and CD8 T cells. The average EC10 calculation for these assays was 1.2 pM for CD4 T cells (range 0.7 to 1.6 pM) and 1.3 pM for CD8 T cells (range 0.9 to 2.0 pM). This assay represents a more conservative estimate of MABEL and was used to estimate the starting dose for patients. For TRI130, 0.7 pM (0.113 ng/ml) was the most conservative approach to MABEL, based on the CD4+ T-cell response from the donors.

Clearance and volume estimates for Group 4 in the single dose NHP study were determined using a WinNonlin (v6.4) precompiled 2 compartment model for intravenous (IV) dosing. Allometric scaling was used to predict human clearance and volume parameter estimates that could be used to simulate dosing strategies that would result in a Cmax below the EC10 (MABEL) value of 0.7 pM, the lowest EC10 determined from an individual donor used in the activation assay. With this modeling, a dose of about 0.005 μg/kg would have a Cmax below the MABEL concentration of 0.113 ng/mL (0.7 pM).

A flat or fixed dose, instead of a weight-based dose is being utilized in this study. Several studies have shown that flat dosing compared to weight-based dosing performed similarly across a number of monoclonal antibodies and that the PK variability introduced by either dosing regimen is moderate relative to the variability generally observed in pharmacodynamics, efficacy, and safety (Wang et al., 2009). Assuming a 60-kg patient and a MABEL dose of 0.005 μg/kg, the starting dose for the study is 0.3 μg.

### Example 2: Administration of TRI130, an Anti-CD123×Anti-CD3 Therapeutic Candidate, to Patients

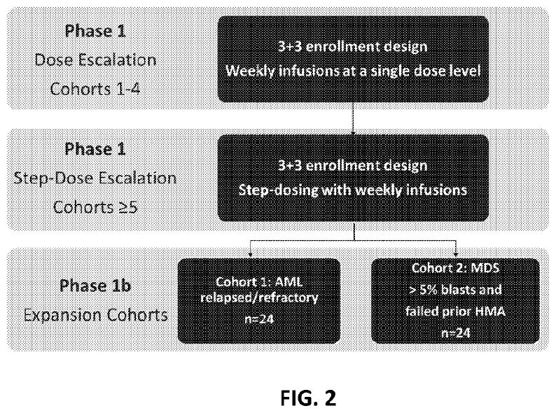

Formulated TRI130 (5 mM succinate, 6.5% sucrose, 0.02% weight/volume polysorbate-80, pH 4.8) is being administered to patients in an ongoing Phase 1/1b open-label, dose-escalation study of patients with relapsed or refractory acute myeloid leukemia (AML) or myelodysplastic syndrome (MDS). The study is being conducted in two parts. The first part is a Phase 1 open-label, dose escalation study to determine the recommended dose for Phase 1b. Phase 1 b is an open-label expansion study to assess the clinical activity and safety of the drug at the recommended dose. The study design is outlined in FIG. 2. Endpoints include safety, immunogenicity, pharmacokinetics, pharmacodynamics, and clinical activity.

Table 8 is a dosing schedule that sets forth the amount that cohorts 1-10 are being dosed weekly (by IV administration) (dose escalation cohorts). In both parts of the study, patients receive drug intravenously weekly for six 28-day cycles, unless disease progression, intolerable toxicity, or withdrawal of consent occurs earlier. There is an option for longer treatment if the patient is responding.

TABLE 8
Dose Escalation Cohorts 1 to 10

| | 1^{st} IV | | 2^{nd} IV | | 3^{rd} IV | | ≥4^{th} IV | | Number of |
|---|---|---|---|---|---|---|---|---|---|
| Cohort | Dose | | Dose | | Dose | | Dose | | Patients |
| 1 | 0.3 | μg | 0.3 | ug | 0.3 | ug | 0.3 | ug | 1 |
| 2 | 1 | μg | 1 | μg | 1 | μg | 1 | μg | 1 |
| 3 | 3 | μg | 3 | μg | 3 | μg | 3 | μg | 1 |
| 4 | 9 | μg | 9 | μg | 9 | μg | 9 | μg | 3 + 3 |
| 5 | 6 | μg | 9 | μg | 12 | μg | 12 | μg | 3 + 3 |
| 6 | Cohort 6 will consist of 2 dose Cohorts | | | | | | | | |
| | conducted concurrently, 6a and 6b | | | | | | | | |
| 6a | 6 | μg | 9 | μg | 12 | μg | 18 | μg | 3 + 3 |
| 6b | 6 | μg | 12 | μg | 12 | μg | 12 | μg | 3 + 3 |
| 7 | 6 | μg | 12 | μg | 18 | μg | 24 | μg | 3 + 3 |
| 8 | 6 | μg | 12 | μg | 18 | μg | 36 | μg | 3 + 3 |
| 9 | 6 | μg | 12 | μg | 18 | μg | 48 | μg | 3 + 3 |
| 10 | 6 | μg | 12 | μg | 18 | μg | 60 | μg | 3 + 3 |

Drug is supplied in single-use vials containing 2 mg drug product in 1 mL of liquid at a concentration of 2 mg/mL. The drug product is mixed with an IV stabilizing solution to prevent drug product from adhering to IV bags and IV tubing sets. The stabilizing solution is supplied sterile and refrigerated (2 to 8° C.) in 10 mL vials comprised of 0.1 M succinate buffer, and 0.08% weight/volume polysorbate 80, at pH 6.0.

For Cohorts 1 through 4, administration is by IV infusion over approximately 20 to 24 hours for the first dose (Cycle 1, Day 1), over 8 hours (±1 hour) for the second dose (Cycle 1, Day 8), over 6 hours (±1 hour) for the third dose (Cycle 1, Day 15) and over 4 hours (±1 hour) for all subsequent doses (Cycle 1, Day 22 and onwards). For Cohorts 5 and above, administration is by IV infusion over 20 to 24 hours for the first dose and every time the dose is increased. The second time the same dose is administered it is infused over 8 hours (±30 minutes), for the third time over 6 hours (±30 minutes), and for the fourth time and all further times over 4 hours (±30 minutes).

If necessary, to manage or prevent any adverse event, and in particular, infusion related reaction (IRR) or cytokine release syndrome (CRS), any dose infusion may be slowed and/or interrupted, with the administration time extended up to 72 hours. If an infusion is extended past 60 hours, then the patient must be observed for 12 hours after the infusion is completed. Table 10 discloses a stepped dosing regimen with the potential to reduce the likelihood of IRR and/or CRS (starting at cohort 5).

The frequency of patient dosing is weekly for up to 6 months. Dosing on a weekly schedule was selected based on the cynomolgus monkey toxicology study of escalating TRI130 doses (data not shown). The half-life of TRI130 following a single dose ranged from approximately 25 to 113 hours for individual animals dosed with 0.25 to 1 mg/kg; the longer half-life estimates were associated with animals in the high dose group (1 mg/kg).

To mitigate infusion related reactions (IRRs) and cytokine release syndrome (CRS), patients are administered the following pre-medications: diphenhydramine, acetaminophen, and dexamethasone. All premedications are administered 1 to 3 hours before the infusion is initiated. The dose of any of the premedications may be reduced, if, in the opinion of the Investigator, comorbidities require it. Dexamethasone is optional after Cycle 2, Day 15, so long as the patient has not experienced any IRRs or CRS with earlier doses. The doses of the 3 premedications are:
1. Dexamethasone 10 to 20 mg IV, or methylprednisolone 1 mg/kg IV, or equivalent; the dose is at the Investigator's discretion based on patient's comorbidities;
2. Acetaminophen 650 or 1,000 mg, or equivalent, orally (PO) three times a day for 1 day (650- or 1,000-mg dose at the Investigators discretion), with the first dose administered 1 to 3 hours before the study drug infusion; and
3. Antihistamine: diphenhydramine 50 mg PO or IV; or equivalent.
If an investigator elects to administer allopurinol for tumor lysis prophylaxis, then it must be started at least 2 days prior to the start of study drug.

### Phase 1—Dose Escalation Study

Dosing was started at the minimum anticipated biologic effect level (MABEL) in patient cohorts. Patients enrolled had either: 1) relapsed or refractory AML and refused or were not eligible for intensive chemotherapy or an allogeneic stem cell transplant, or 2) relapsed or refractory MDS and had >5% blasts in the marrow or any circulating blasts in the peripheral blood and had failed a prior hypomethylating agent (HMA); failure is defined as intolerance to HMA, lack of response (no CR by at least 6 cycles), or IWG defined progressive disease during or after treatment with an HMA. Demographics for 32 patients enrolled in the ongoing Phase 1 dose escalation study (through Cohort 7) are shown below in Table 9. For patients enrolled through Cohort 7, the median age was 67 years, and 79% of the patients had AML. The average number of doses administered per patient was 8.5 with an average treatment duration of 54 days.

TABLE 9
Demographics and exposure, patients enrolled through cohort 7

| | |
|---|---|
| Statistic | All Patients (N = 32) |

Characteristic

| | | | |
|---|---|---|---|
| Age | Median (Range) | 67 | (18, 81) |
| Female | N (%) | 15 | (47%) |
| Prior Lines of Therapy | Median (Range) | 3 | (1, 13) |

Disease

| | | | |
|---|---|---|---|
| MDS | N (%) | 7 | (22%) |
| AML Primary | N (%) | 21 | (66%) |
| AML Therapy Related | N (%) | 4 | (13%) |

Cytogenetic Risk

| | | | |
|---|---|---|---|
| Favorable | N (%) | 3 | (9%) |
| Intermediate | N (%) | 7 | (22%) |
| Poor | N (%) | 6 | (19%) |
| Unknown | N (%) | 16 | (50%) |

| Summary of Treatment Exposure | 32 Patients Mean (SD) | |
|---|---|---|
| Doses Administered | 8.5 | (8.3) |
| Treatment Duration (days) | 54 | (59) |

Treatment-Related Adverse Events for the 32 patients enrolled in the ongoing Phase 1 dose escalation study are shown below in Table 10. 34% of patients experienced one or more IRR/CRS events (Grade≥3 reported in 16%). The most common symptoms were dyspnea, fever, hypotension, hypoxia, tachycardia, and rigors/chills. Notably, IRR/CRS was the only treatment-related serious adverse event to occur in two or more patients. 3 out of the 11 patients that experienced an IRR/CRS event received tocilizumab to treat the same.

TABLE 10
Treatment-Related Adverse Events

| | |
|---|---|
| All (N = 32) | Grade ≥ 3 (N = 32) |

Treatment-Related Adverse Events*

| | | | | |
|---|---|---|---|---|
| IRR/CRS** | 11 | (34%) | 5 | (16%) |
| Fatigue | 5 | (16%) | 1 | (3%) |
| Diarrhea | 3 | (9%) | | |
| Fever | 3 | (9%) | | |
| Anemia | 2 | (6%) | 2 | (6%) |
| Flushing | 2 | (6%) | | |
| Hypotension | 2 | (6%) | | |
| Nausea | 2 | (6%) | 1 | (3%) |
| Peripheral Edema | 2 | (6%) | | |
| Rigors/Chills | 2 | (6%) | | |

Treatment-Related Serious Adverse Events

| | | | | |
|---|---|---|---|---|
| IRR/CRS | 8 | (25%) | 4 | (13%) |

*Treatment-related AE and SAE events occurring in 2 or more patients
**Infusion Related Reaction/Cytokine Release Syndrome
Toxicity grading is based on CTCAE criteria version 5.0

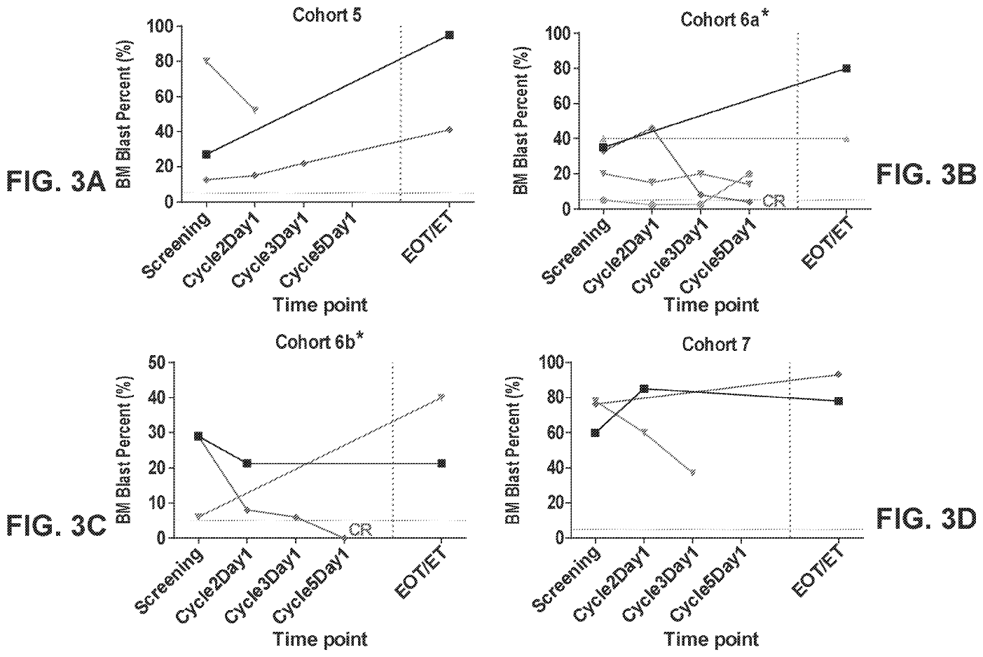

The percentage of blasts in bone marrow aspirates was monitored overtime for patients. As shown in FIG. 3A-3D, a reduction of bone marrow blasts was observed in several patients that received a highest dose≥12 μg. Two patients each had reductions in bone marrow blasts from 29% to 0% (Cohort 6b, FIG. 3C), and from 33% to 4% (Cohort 6a, FIG. 3B). Absolute neutrophil and platelet counts met Complete Remission criteria. Both patients remain on study, as well as one patient with decreasing blasts in Cohort 7.

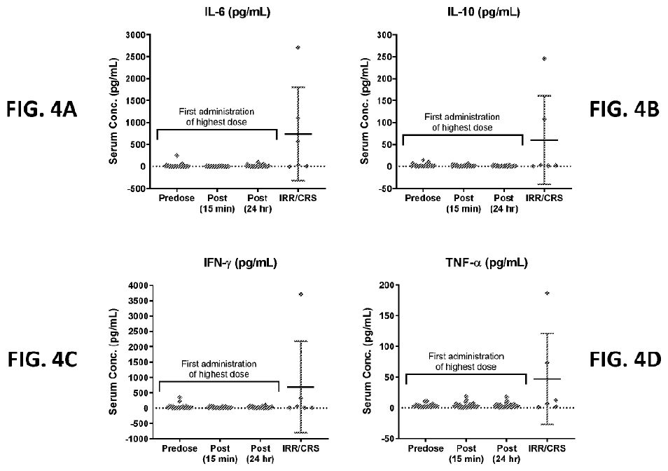

Serum cytokines were assessed at scheduled timepoints before and after administration of the highest dose in each patient, and were also evaluated at intervals during infusion related reactions or cytokine release syndrome events. As shown in FIG. 4A-4D, cytokines were not elevated during scheduled collections. Elevated cytokines, particularly IL-6, were observed during adverse events of IRR/CRS. Notably, in this small data set, no correlation was observed between the highest cytokine concentrations and dose level or grade of event.

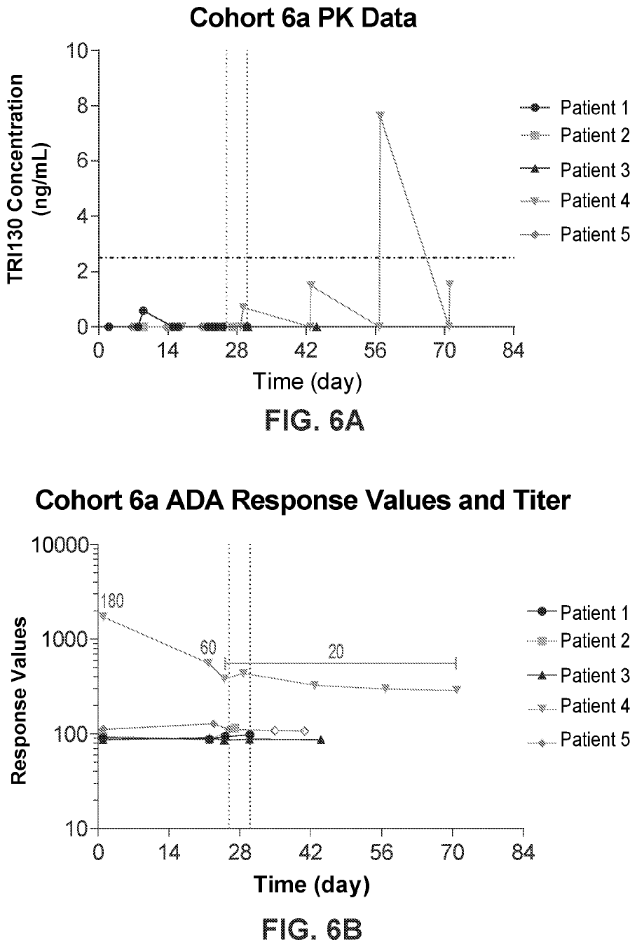

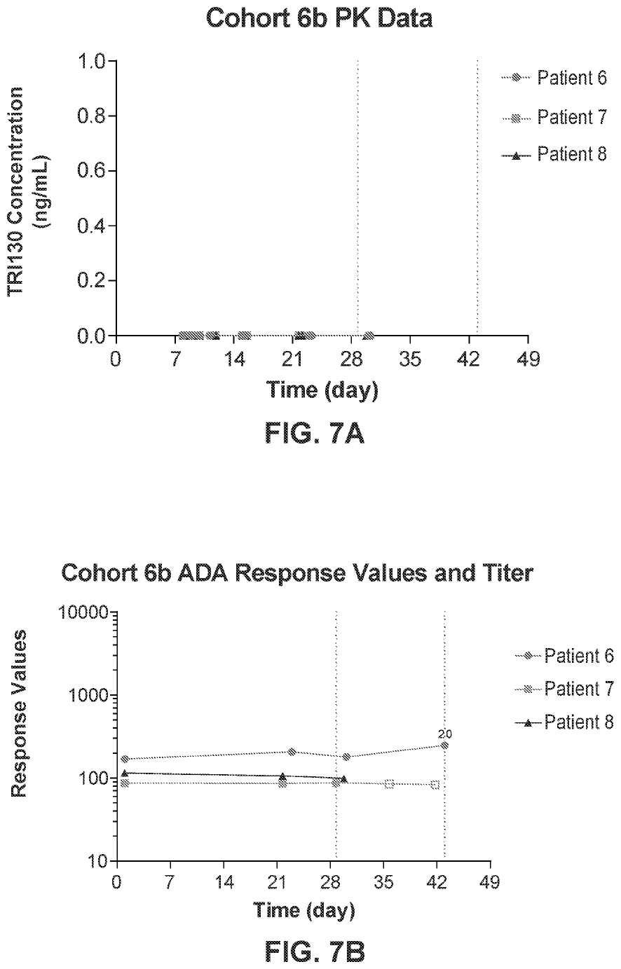

Pharmacokinetic (PK) data was analyzed for patients in Cohorts 6A (FIG. 6A) and 6B (FIG. 7A). In Cohort 6A, quantifiable concentrations of TRI130 were seen post C3D1 (Day 56) for one patient. All other samples were below the limit of quantitation (BLQ) for the current assay format (2.5 ng/mL), but multiple samples were in the detectable range of the assay.

Anti-drug antibody levels were also determined for patients in Cohorts 6A (FIG. 6B) and 6B (FIG. 7B). In Cohort 6A, one patient (Patient 4) was screened and confirmed positive, however titers were low. Also, the patient was positive for ADAs pre-dose, and the titer of such antibodies decreased over time, indicating that the response was not treatment related. In Cohort 6B, Patient 6 measured post-dose positive for anti-TRI130 antibodies. In this patient, titer was also low. Notably, the pre-dose background was below the cut-off point, but was relatively high compared to other negative individuals, indicating that this response may not be related to the TRI130 treatment.

Thus, in this preliminary study, administration of TRI130, through doses of 24 μg, was tolerated with a manageable safety profile. As noted above, two patients had complete remission (CR). Cytokines were not significantly elevated unless there was a concurrent adverse event of IRR/CRS. Preliminary data suggested no evidence of treatment-induced anti-drug antibodies (ADA).

This open-label, multiple-dose ascending dose escalation phase was designed to determine the recommended dose level of TRI130 for future Phase 2 studies, specifically, a dose of TRI130 that does not produce any acute, clinically-significant cytokine release that poses a safety risk for patients. This stage of the trial demonstrated TRI130 has a manageable safety profile and established the recommended Phase 2 dose (“RP2D”). Further, pharmacokinetic (PK) data from the dose escalation study indicated that treatment as a single agent at the RP2D level, which is below the maximum tolerated dose (“MTD”) level, achieved prolonged stabilization of leukemia, a response that consequently deepened to partial remission and complete remission (“CR”) in two difficult to treat relapsed/refractory AML patients. The infusion-related reactions and cytokine release syndrome cases experienced in part one of the Phase 1B study were manageable with dose interruption, administration of dexamethasone and/or tocilizumab.

### Phase 1—Additional Dose-Escalation Cohorts

After completion of a dose-limiting toxicity (DLT) observation period for Cohort 7, four additional sequential cohorts (Cohorts A, B, C, and D) will enroll patients while Cohorts≥8 are treated. These cohorts will run sequentially and are independent of Cohorts 8 to 10, and will achieve a more rapid dose escalation.

Patients in Cohorts A, B, C, and D will have continuous IV dosing (20-24 hours/day) for the first 4 days of Cycle 1, then twice a week dosing during Week 2, followed by once a week dosing thereafter in Cycle 1 and all subsequent cycles. Table 11 shows the dosing schedule for Cohorts A, B, C, and D. The first week of dosing in Cohort A utilizes doses tested in Cohort 6a (Day 1 of 6 μg, Day 2 of 9 μg, Day 3 of 12 μg, and Day 4 of 18 μg). During the second week, the dose of 18 μg is administered on Day 8 and Day 11. In Week 3 the dose is escalated to 36 μg and is maintained at that level. The advantage of escalating the daily dose during the first week is that the Cₘₐₓ is gradually increased, which may lower the propensity for the development of IRR/CRS. Aggressive treatment with tocilizumab will be administered for Grade≥2 IRR or CRS that does not respond within 2 hours of symptomatic treatment and dose interruption.

TABLE 11
Dosing for Cohorts A, B, C and D

| | | | | | | | | ≥Cycle | |
|---|---|---|---|---|---|---|---|---|---|
| | | | | | | | | 2 | |
| | | | | | | | Weeks | Days | |
| | Week 1 | | | | Week 2 | | 3 & 4 | 1, 8, | Number |
| | Day | Day | Day | Day | Day | Day | Days | 15, | of |
| Cohort | 1 | 2 | 3 | 4 | 8 | 11 | 15, & 22 | and 22 | Patients |
| A | 6 μg | 9 μg | 12 μg | 18 μg | 18 μg | 18 μg | 36 μg | 36 μg | 3 + 3 |
| B | 6 μg | 12 μg | 18 μg | 24 μg | 24 μg | 24 μg | 48 μg | 48 μg | 3 + 3 |
| C | 6 μg | 12 μg | 24 μg | 36 μg | 36 μg | 36 μg | 60 μg | 60 μg | 3 + 3 |
| D | 6 μg | 12 μg | 24 μg | 36 μg | 48 μg | 48 μg | 100 μg | 100 μg | 3 + 3 |

For Cohorts A-D, administration is by IV infusion over 20 to 24 hours for the first dose and every time the dose is increased. The second time the same dose is administered it is infused over 8 hours (±30 minutes), for the third time over 6 hours (±30 minutes), and for the fourth time and all further times over 4 hours (±30 minutes).

Premedications are administered before Cycle 1, Day 1; Cycle 1, Day 8; Cycle 1, Day 11; Cycle 1, Day 15; and Cycle 1, Day 22. For all subsequent doses, dexamethasone is optional, but acetaminophen and diphenhydramine are required.

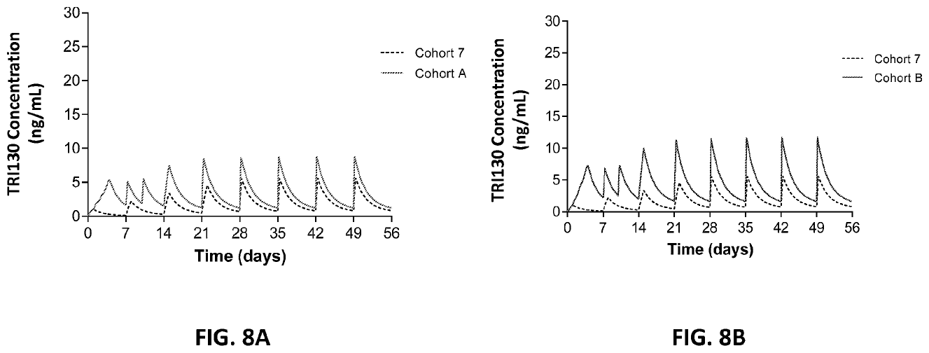

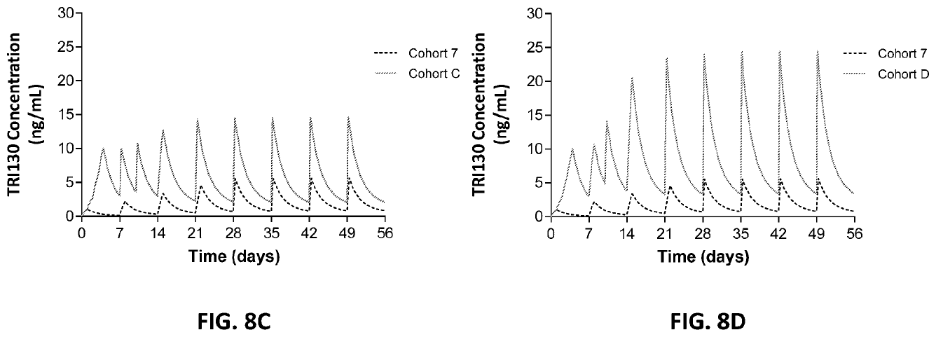

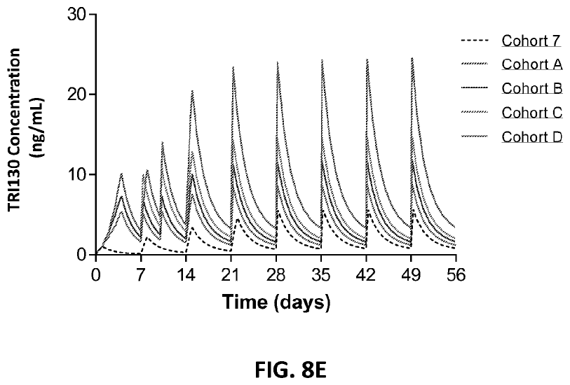

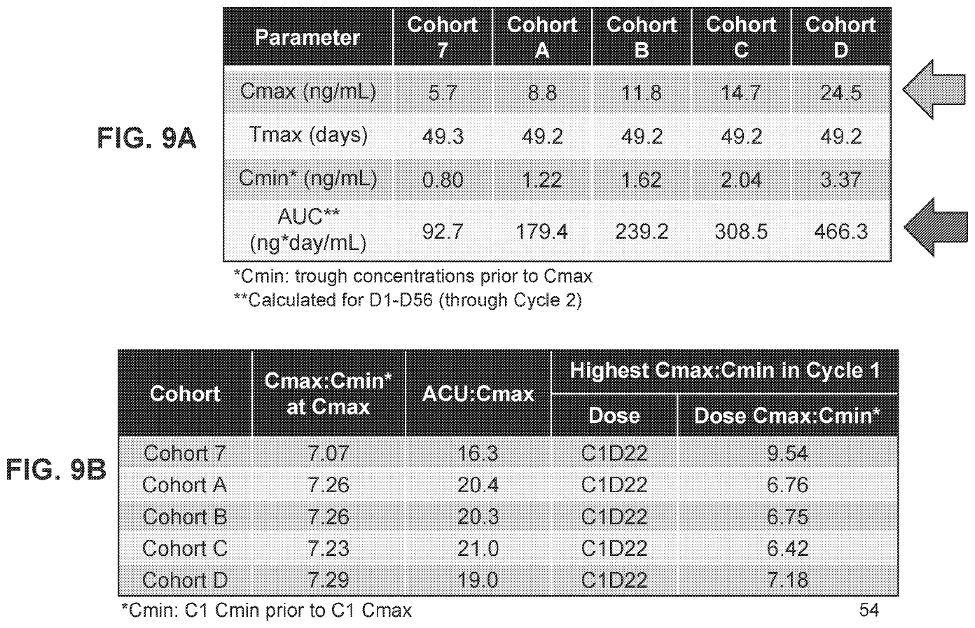

Provided in FIG. 8A-8E are PK simulations for each of Cohort A, B, C, and C, which show how the Cₘₐₓ is gradually increased over time. Simulations for additional PK parameters are also provided in FIGS. 9A and 9B. These simulations were based on modeling of low dose date in cynomolgus monkeys, to account for limited amount of TMDD (target-mediated drug disposition).

### Example 3: Phase 1b Expansion Study of TRI130 in AML and MDS Patients

The maximum-tolerated dose (MTD) for TRI130 was not reached at a dose level of 240 μg/cycle (Cohort 10 in Table 10) in the dose escalation study described in Example 2. The sub-MTD dose level of Cohort 6A was identified as the recommended phase 2 dose (RP2D) level of TRI130 for further evaluation during the expansion phase. This dose level provides more than 50% lower amount of TRI130 than what was administered in Cycle 1 of Cohort 10 without dose-limiting toxicities (DLTs) (viz., 45 μg vs. 96 μg in Cycle 1) and 70% lower than the dose level in Cohort 10 in second and subsequent cycles (72 μg vs. 240 μg in Cycles 2-4). Both prolonged stable disease for greater than 6 months and complete remission were observed as best overall responses to TRI130 as a single agent at this sub-MTD dose level that was selected for further testing during the expansion phase.

Accordingly, in this Phase 1 b Expansion Study, the dose level that was used in Cohort 6A of the dose escalation phase (Example 2) will be used to evaluate the safety and tolerability of TRI130 at the RP2D level when used as an adjunct to the standard of care. The anti-leukemia activity of TRI130 will be assessed as monotherapy as well as in combination with standard chemotherapy drugs during this expansion phase.

This study is an open-label, multi-center, dose expansion study that will enroll a total of 90 primary AML patients into five cohorts of 18 patients each. The treatment arms are designed to evaluate safety and efficacy endpoints. In Cohorts 1-4, TRI130 will be administered at a fixed dosage of 18 μg after a weekly ramp up during Cycle 1 (Cohorts 1, 3, 4) or Cycle 1-2 (Cohort 2). In Cohort 5, TRI130 will be administered at a fixed dose of 18 μg twice weekly after a weekly ramp up during Cycle 1. Each of these cohorts are described in further detail below.

Cohort 1—Induction with Chemotherapy Plus TRI130

Patients will be treated with a combination of chemotherapy (ChT) plus TRI130. For ChT, patients may receive either cytarabine intermediate dose (IDAC) or a combination of mitoxantrone, etoposide, and cytarabine (MEC).

Primary or secondary AML patients (Age: >18 years) in 1st or 2nd relapse with last complete remission (CR)<12 months or primary refractory disease will receive 4×28-day cycles of combined 2-drug immunochemotherapy, comprising (i) a combination of TRI130 and intermediate dose cytarabine (IDAC), or (ii) a combination of TRI130 and MEC (mitoxantrone, etoposide, cytarabine). Primary refractory disease is defined by the European LeukemiaNet (ELN) as the inability to attain CR or complete remission with incomplete hematologic recovery (CRi) after two courses of intensive induction treatment.

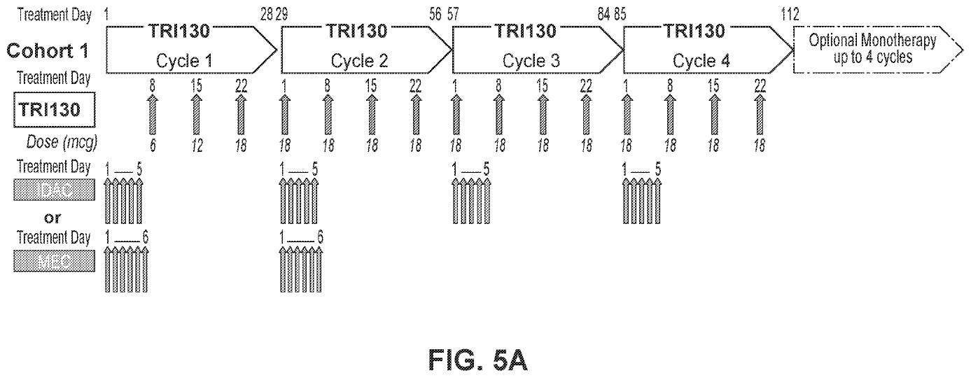

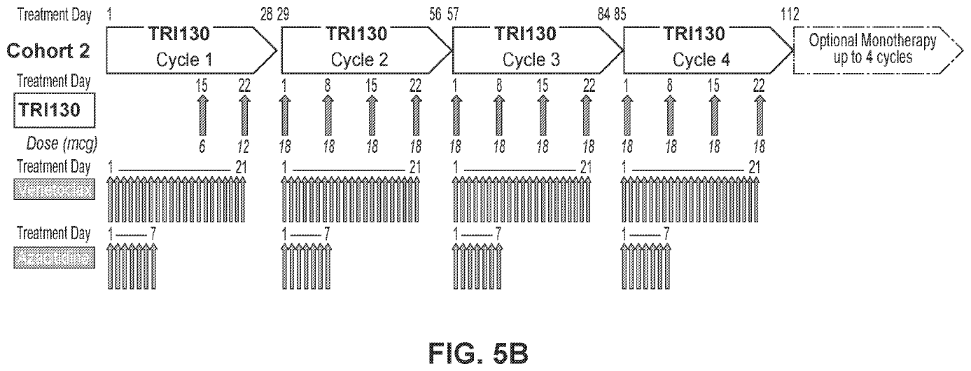

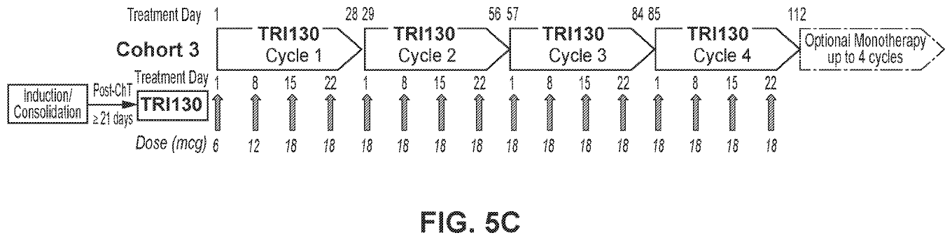

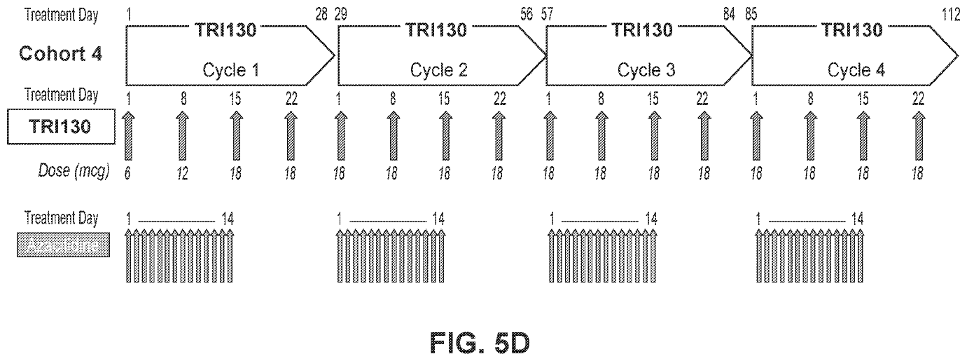

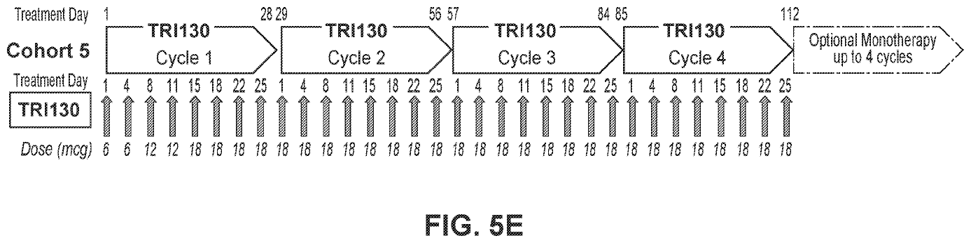

If IDAC is chosen as the ChT, patients will receive 4 cycles of IDAC in combination with TRI130. Cytarabine (1 g/m²) will be administered intravenously over 2 hours daily on days 1-5 during of each 28-day cycle (D1-D5). On day 1 of Cycle 2 (D29), Cycle 3 (D57) and Cycle 4 (D85) cytarabine will be administered 4 hours after the end of the TRI130 infusion. This dosing regimen is illustrated in FIG. 5A.

If MEC is chosen as the ChT, patients will receive 2 cycles of mitoxantrone 6 mg/m²/day IV on days 1-6, etoposide 80 mg/m²/day IV on days 1-6, and cytarabine 1 g/m²/day IV on days 1-6. TRI130 will be administered in combination with MEC for 2 cycles and subsequently as monotherapy for 2 cycles. This dosing regimen is illustrated in FIG. 5A.

Cycle 1, Day 1 (C1D1) for TRI130 will be day 8 after initiation of IDAC or MEC chemotherapy. The first TRI130 cycle will include only 3 doses of TRI130 (Cl D1, C1D8, C1D15) separated by one week. All subsequent cycles will have 4 weekly infusions of TRI130. TRI130 will be administered intravenously over 4 hours on days 1, 8, 15, and 22 of each TRI130 cycle at a fixed dosage of 18 μg after a weekly ramp up during Cycle 1 of TRI130. Step-up dosing will proceed as shown in Table 12.

TABLE 12
Step-up dosing

| | Dose of | | Infusion | | Observation period | |
|---|---|---|---|---|---|---|
| Day of treatment | TRI130 | | time | | post-dose | |
| D8 = C1D1 of | 6 | μg | 20-24 | hours | 24 | hours |
| TRI130 calendar | | | | | | |
| D15 = C1D8 of | 12 | μg | 20-24 | hours | 24 | hours |
| TRI130 calendar | | | | | | |
| D22 = C1D15 of | 18 | μg | 20-24 | hours | 24 | hours |
| TRI130 calendar | | | | | | |
| D29 = C2D1 of | 18 | μg | 8 | hours | 4 | hours |
| TRI130 calendar | | | | | | |
| D36 = C2D8 of | 18 | μg | 6 | hours | 2 | hours |
| TRI130 calendar | | | | | | |
| all subsequent | 18 | μg | 4 | hours | 2 | hours |
| doses | | | | | | |

The primary outcome measures for Cohort 1 are (1) For Safety: the cumulative incidence of Grade 3-4 adverse events (AEs), and severe adverse events (SAEs), and the incidence of AEs of interest (≥Grade 2 cytokine release syndrome (CRS), ≥Grade 2 Infusion related reaction, ≥2 cardiac toxicity and ≥2 neurotoxicity as complications of CRS) for safety and (2) For Efficacy: Leukemia-free survival (LFS); composite CR rate (CR/CRi/CRh) after each cycle, and minimum residual disease (MRD) status by multi-color/multi-parameter flow cytometry (MFC) by Central Lab determination for patients who achieve CR.

Cohort 2—Induction with TRI130+Venetoclax+Azacitidine—Frontline Or 1^{st} Relapse

Poor prognostic but primary or secondary AML patients (Age>18 years) who are treatment-naïve or in 1st relapse will receive 4×28-day cycles of combined 3-drug immunochemotherapy comprising TRI130 plus Venetoclax and Azacitidine. A minimum of 15 days must have passed since the first dose of Venetoclax to minimize the risk of tumor lysis syndrome (TLS) while patients start TRI130. The first TRI130 dose in the first TRI130 cycle will be administered 15 days after the first dose of Venetoclax. In subsequent cycles, the first dose of TRI130 and Venetoclax can be administered on the same day without a 15-day separation.

TRI130 will be administered intravenously over 4 hours weekly on days 1, 8, 15, and 22 of each TRI130 cycle at a fixed dosage of 18 μg after a weekly ramp up during Cycle 1 of TRI130. Step-up dosing will proceed as shown in Table 13.

TABLE 13
Step-up dosing

| | Dose of | | Infusion | | Observation period | |
|---|---|---|---|---|---|---|
| Day of treatment | TRI130 | | time | | post-dose | |
| C1D15 according | 6 | μg | 20-24 | hours | 24 | hours |
| to Venetoclax | | | | | | |
| calendar (C1D1 | | | | | | |
| according to | | | | | | |
| TRI130 calendar) | | | | | | |
| C2D22 according | 12 | μg | 20-24 | hours | 24 | hours |
| to Venetoclax | | | | | | |
| calendar | | | | | | |
| C2D8 according | 18 | μg | 8 | hours | 4 | hours |
| to Venetoclax | | | | | | |
| calendar | | | | | | |
| all subsequent | 18 | μg | 4 | hours | 2 | hours |
| doses | | | | | | |

Venetoclax will be administered daily, orally on days 1-21 of each cycle at a fixed dosage of 400 mg/daily after a daily ramp up (Day 1: 100 mg; Day 2: 200 mg; Day 3-Day 21: 400 mg). Venetoclax dose will be adjusted per standard of care for patients on azole anti-fungals. Venetoclax may be given Days 1-14 only after Cycle 2 for patients with <5% bone marrow blasts.

Azacitidine will be administered intravenously over 30 minutes daily on days 1-7 of each cycle at a dosage of 75 mg/m².

The dosing regimen for Cohort 2 is illustrated in FIG. 5B.

The primary outcome measures for Cohort 2 are (1) For Safety: the cumulative incidence of Grade 3-4 AEs, and SAEs, and the incidence of AES of interest (≥Grade 2 CRS, ≥Grade 2 Infusion related reaction, ≥2 cardiac toxicity and ≥2 neurotoxicity as complications of CRS) for safety and (2) For Efficacy: Leukemia-free survival (LFS); composite CR rate (CR/CRi/CRh) after each cycle, and MRD status by MFC (Central Laboratory) for patients who achieve CR. HSCT eligible patients can undergo HSCT after 2 cycles of the triplet APVA if in CR and MRD-negative by flow cytometry. Otherwise, 4 TRI130 cycles per protocol strongly encouraged. After the 4 cycles of TRI130+Venetoclax+Azacitidine triple therapy, patients will continue Venetoclax+Azacitidine in follow-up per discretion of the Investigator. Patients who achieve complete remission plus minimum residual disease (CRMRD) status after 4 cycles of TRI130 but are not eligible to undergo hematopoietic stem cell transplantation (HSCT) may receive additional 4 TRI130 monotherapy cycles as per Investigator preference.

### Cohort 3—Consolidation Post 7+3—Frontline+1^{st} Relapse

Primary AML patients (Age: >18 years) with FLT3-negative intermediate or adverse risk AML (including but not limited to: TP53, RUNX1 and ASXL1 mutations and/or complex cytogenetics) who are treatment-naïve or in 1st relapse with a duration of CR1<1 year will receive 4×28-day cycles of immunotherapy with TRI130 after hematologic recovery (ANC>1,000/μL; Hgb≥9 g/dL; Plt?100,000/μL) post induction with either (a) or (b) as described below.

(a) For Newly diagnosed patients: standard 7+3 therapy consisting of: Cytarabine 100-200 mg/m² administered intravenously daily as a continuous infusion for 7 consecutive days on days 1-7 days plus; Idarubicin 12 mg/m² administered intravenously over 15 min on days 1-3 (or Daunorubicin 60 [30-90] mg/m² intravenous over 3 hours). CPX-351 induction at standard dose and schedule is also acceptable if preferred by the Investigator. Patients receiving 1 cycle of high dose cytarabine (HiDAC) consolidation or 1-2 cycles of IDAC or CPX-351 post 7+3 induction therapy are eligible (HiDAC may be reduced to 1.5 grams/m² for patients>60 years of age per institutional practices and/or discretion of the Investigator). Patients with ≥25% blasts in bone marrow after induction 1 (blast persistence) should receive a second induction cycle, which may but does not need to be the identical ChT as induction 1. As soon as patients achieve CR/CRi after 1 or 2 induction cycles, they should proceed to consolidation treatment.

(b) For Patients in 1st Relapse, a cytarabine-based standard salvage regimen may also be used such as: Mitoxantrone+Etoposide+Cytarabine (MEC). If treated with MEC, patients will receive mitoxantrone 6 mg/m²/day IV on days 1-6, etoposide 80 mg/m²/day IV on days 1-6, and cytarabine 1 g/m²/day IV on days 1-6. FLAG or FLAG-IDA should not be used due to the potential risk of fludarabine-associated neurotoxicity and autoimmune complications being enhanced by TRI130.

A minimum of 21 days must have passed since the last Cytarabine dose to allow for resolution of the side effects associated with the 7+3 induction or HiDAC consolidation. TRI130 should be started after the final contemplated ChT cycle. Patients must meet all eligibility criteria in order to receive immunotherapy with TRI130. Patients are required to be either not in CR or be in CR is MRD positive (01% level) by MFC (Central Laboratory) post induction/consolidation to be eligible for TRI130 treatments.

The first TRI130 consolidation cycle will start≥21 days (preferably 21-28 days) post 7+3 or MEG induction or HiDAC/IDA containing consolidation as per investigator preference.

TRI130 will be administered intravenously over 4 hours weekly on days 1, 8, 15, and 22 of each TRI130 consolidation cycle at a fixed dosage of 18 μg after a weekly ramp up during Cycle 1. Step-up dosing will proceed as shown in Table 14.

TABLE 14
Step-up dosing

| | Dose of | | Infusion | | Observation period | |
|---|---|---|---|---|---|---|
| Day of treatment | TRI130 | | time | | post-dose | |
| C1D1 - Day 1 of | 6 | μg | 20-24 | hours | 24 | hours |
| TRI130 Cycle 1: | | | | | | |
| Cycle 1 starts on | | | | | | |
| Day 22 (up to Day | | | | | | |
| 36) of first HiDAC | | | | | | |
| consolidation | | | | | | |
| C1D8 | 12 | μg | 20-24 | hours | 24 | hours |
| C1D15 | 18 | μg | 20-24 | hours | 4 | hours |
| C1D22 | 18 | μg | 8 | hours | 4 | hours |
| C2D21 (occurs after | 18 | μg | 6 | hours | 2 | hours |
| 2nd cycle of HiDAC | | | | | | |
| consolidation | | | | | | |
| starting Day 22-36) | | | | | | |
| all subsequent | 18 | μg | 4 | hours | 2 | hours |
| doses | | | | | | |

The dosing regimen for Cohort 3 is illustrated in FIG. 5C.

The primary outcome measures for Cohort 3 are (1) For Safety: the cumulative incidence of Grade 3-4 AEs, and SAEs, and the incidence of AES of interest (≥Grade 2 CRS, ≥Grade 2 Infusion related reaction, ≥2 cardiac toxicity and ≥2 neurotoxicity as complications of CRS) for safety and (2) For Efficacy: Leukemia-free survival (LFS); MRD status by MEG for patients who are in remission at the start of TRI130 consolidation, and composite CR rate for patients who are not in CR/CRi/CRh at the beginning of TRI130 consolidations.

Eligible patients can undergo HSCT after 2 cycles of TRI130 if in CR and MRD-negative by MFC. Otherwise, 4 TRI130 cycles per protocol strongly encouraged.

Patients who achieve CR_{MRD}-status after 4 cycles of TRI130, but are not eligible to undergo HSCT, may receive additional 4 TRI130 monotherapy cycles as per physician preference.

### Cohort 4—MRd-Positive (MRD+) 1^{st} Remission, TRI130+Oral Azacitidine

Patients greater than 18 years old with minimum residual disease (at ≥0.1% level by multicolor-multiparameter flow cytometry [MFC] in Central Lab), high-risk 1st remission AML patients will be treated with 4×28-day cycles of TRI130+oral azacitidine (Onureg, CC-486).

1st remission AML patients who received frontline standard 7+3 therapy or other standard induction therapy, including but not limited to hypomethylating agents plus venetoclax or low dose cytarabine plus venetoclax, are eligible. Patients receiving 1 cycle of high dose cytarabine (HiDAC) consolidation or 1-2 cycles of IDAC or CPX-351 post 7+3 induction therapy are eligible if they have MRD+CR.

Patients in MRD+1st remission after a minimum of 4 cycles of venetoclax-based therapy are eligible.

Cycle 1, Dose 1 of TRI130 must be ≥21 days post HiDAC or IDAC-containing consolidation. TRI130 will be administered intravenously over 4 hours weekly on days 1, 8, 15, and 22 of each cycle at a fixed dosage of 18 μg after a weekly ramp up during Cycle 1. Step-up dosing will proceed as shown in Table 15.

TABLE 15
Step-up dosing

| | Dose of | | Infusion | | Observation period | |
|---|---|---|---|---|---|---|
| Day of treatment | TRI130 | | time | | post-dose | |
| C1D1 - Day 1 of | 6 | μg | 20-24 | hours | 24 | hours |
| Cycle 1 of TRI130 | | | | | | |
| C1D8 | 12 | μg | 20-24 | hours | 24 | hours |
| C1D15 | 18 | μg | 20-24 | hours | 4 | hours |
| C1D22 | 18 | μg | 8 | hours | 4 | hours |
| C2D1 | 18 | μg | 6 | hours | 2 | hours |
| all subsequent | 18 | μg | 4 | hours | 2 | hours |
| doses | | | | | | |

Oral azacitidine will be administered at 300 mg daily dose level×14 days. The dosing regimen for Cohort 4 is illustrated in FIG. 5D.

Eligible patients can undergo HSCT after 2 cycles of TRI130+azacitidine if they achieve MRD-status by MFC. Otherwise, 4 cycles of TRI130+azacitidine per protocol strongly encouraged. Patients who achieve CRMRD-status after 4 cycles of TRI130, but are not eligible to undergo HSCT, may receive additional 4 TRI130 monotherapy cycles as per Investigator preference.

### Cohort 5—MRD+2^{nd} Remission, Single Agent TRI130

AML patients greater than 18 years old, MRD+(at ≥0.1% level by MFC in Central Lab) who are in 2nd remission post-induction with a standard of care regimen will be treated with 4×28-day cycles of TRI130 monotherapy.

Patients with, as well as without, post-induction consolidation with a standard of care regimen are eligible. Patient enrollment will be staggered in the first 5 patients (=Safety Lead in) with at least 7 days in between the consecutive patients 1-5.

Cycle 1, Dose 1 of TRI130 must be ≥21 days post HiDAC or IDAC-containing consolidation. TRI130 will be administered intravenously over 4 hours twice weekly on days 1, 4, 8, 11, 15, 18, and 22 of each cycle at a fixed dosage of 18 μg after a weekly ramp up during Cycle 1 (Step-up dosing: TRI130 will be administered intravenously over 4 hours weekly on days 1, 8, 15, and 22 of each cycle at a fixed dosage of 18 μg after a weekly ramp up during Cycle 1.

Step-up dosing will proceed as shown in Table 16.

TABLE 16
Step-up dosing

| | Dose of | | Infusion | | Observation period | |
|---|---|---|---|---|---|---|
| Day of treatment | TRI130 | | time | | post-dose | |
| C1D1 and C1D4 | 6 | μg | 20-24 | hours | 24 | hours |
| C1D8 and C1D11 | 12 | μg | 20-24 | hours | 24 | hours |
| C1D15 and C1D18 | 18 | μg | 20-24 | hours | 4 | hours |
| C1D22 | 18 | μg | 8 | hours | 4 | hours |
| C2D1 | 18 | μg | 6 | hours | 2 | hours |
| all subsequent | 18 | μg | 4 | hours | 2 | hours |
| doses | | | | | | |

The dosing regimen for Cohort 5 is illustrated in FIG. 5E.

If the twice weekly dosing results in Grade 3 IRR or CRS during the safety lead in, the C1D4 and C1 D11 doses of TRI130 may be omitted.

Eligible patients can undergo HSCT after TRI130 after 2 cycles of TRI130 if in CR and MRD-negative by FCM. Otherwise, 4 cycles per protocol strongly encouraged. Patients who achieve CRMRD-status after 4 cycles of TRI130 may receive additional 4 TRI130 monotherapy cycles as per Investigator preference.

### Example 4: Single Agent Activity of TRI130 in Patients with Relapsed/Refractory AML or MDS

The primary purpose of this proof of concept study was to evaluate 8 patients with relapsed or refractory (R/R) AML who had failed treatment with hypomethylating agents (HMA, N=2) or venetoclax plus HMA (N=6) and 6 patients with R/R MDS who had failed treatment with HMA (N=5) or venetoclax plus HMA (N=1). The clinical activity of TRI130 was tested at submicrogram dose levels>0.08 μg/kg that were active in preclinical NOD/SCID mouse xenograft models of AML. This analysis evaluated clinical proof of concept performed using the primary data from a recently completed Phase 1B study of TRI130 in R/R AML and MDS patients.

### Clinical Study

This proof-of-concept study was performed under IND 135552 as part of a multiinstitutional Phase 1B clinical dose escalation trial of TRI130 in patients with relapsed/refractory AML and higher-risk myelodysplastic syndrome (MDS). It was registered in the clinical trial database ClinicalTrials.gov with the identifier number NCT03647800. The weekly target dose levels for cohorts 2-10 were 1 μg for Cohort 2, 3 mcg for Cohort 3, 9 mcg for Cohort 4, 18 mcg for Cohort 6A, 12 mcg for Cohort 6B, 24 mcg for Cohort 7, 36 mcg for Cohort 8, 48 mcg for Cohort 9, and 60 mcg for Cohort 10 [28] (See Example 2, Table 10 above). A 3+3 design was used to guide the dose escalation. In each cohort, eligible AML/MDS patients were assigned to receive a designated flat dose of TRI130 as a single agent via weekly intravenous infusions. TRI130 was administered at the indicated dose levels as a single agent via weekly intravenous infusions [28]. TRI130 was administered according to an intra-patient step-up strategy to reduce the risk for cytokine release syndrome (CRS) that was implemented in cohort 5 with a protocol amendment. DNA sequencing for molecular profiling of leukemic blast cells was performed using the Genoptix (Carlsbad, CA) platform. All responses were assessed by ELN 2017 criteria for AML and IWG 2006 criteria for MDS. TRI130 exhibited a favorable safety profile with acceptable tolerability and manageable treatment-emergent AEs [28]. The MTD was not reached at a weekly flat dose of 60 μg (1 μg/kg for a 60-kg subject) [28]. The most common grade 3 AEs suspected to be TRI130-related were grade 3-4 CRS occurring in 4 of 46 patients (8.7%), grade 3-4 anemia occurring in 2 of 46 patients (4.3%), and infusion-related reactions (IRR) occurring in 2 of 26 patients (4.3%) [28].

### Measurement of Serum Cytokine Levels and Flow Cytometry

The Meso Scale Discovery (MSD) U-PLEX assay platform and a MSD Meso Quickplex SQ 120 Reader Instrument (Meso Scale Diagnostics, Rockville, MD) were used in a central laboratory setup for measurement of serum levels of the proinflammatory cytokines interleukin-5 (IL-5), interleukin-10 (IL-10), and interferon-gamma (IFN-γ) by electrochemiluminescence in duplicate serum samples. Immunophenotyping was performed on cryopreserved peripheral blood mononuclear cells from patients by standard flow cytometry using a BD LSR II flow cytometer and FACSDiva Software Version 8.0.2 fluorochrome-labeled monoclonal antibodies reactive with CD5 (anti-human CD5, clone REA782 [PE-Vio770), CD45 (anti-human CD45, Clone H130, V500, BD Biosciences #560777), CD34 (anti-human CD34, Clone REA1164, VioBright 515, Miltenyl Biotech #130-120-517) and CD123 (anti-human CD123, Clone 9F5, AF647, BD Biosciences #563599) antigens.

### Statistical Analyses

Standard statistical methods were applied for the analysis of the clinical data. Survival data was analyzed by the Kaplan-Meier method using the GraphPad Prism 9 statistical program (GraphPad Software, LLC, San Diego, CA). Log-rank statistics was used to compare the differences between patient subgroups [29,30].

### Results

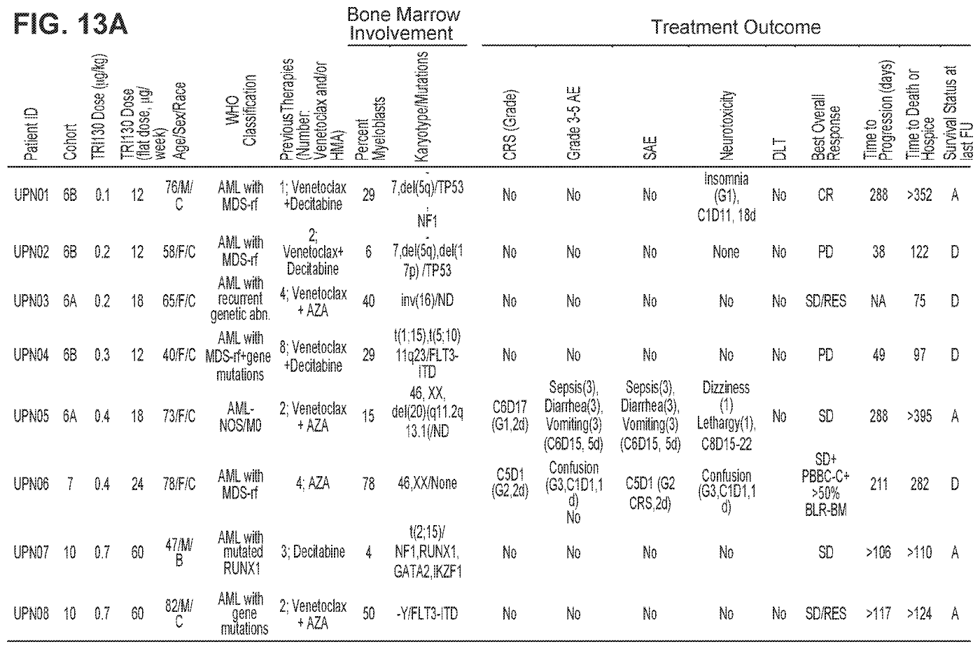

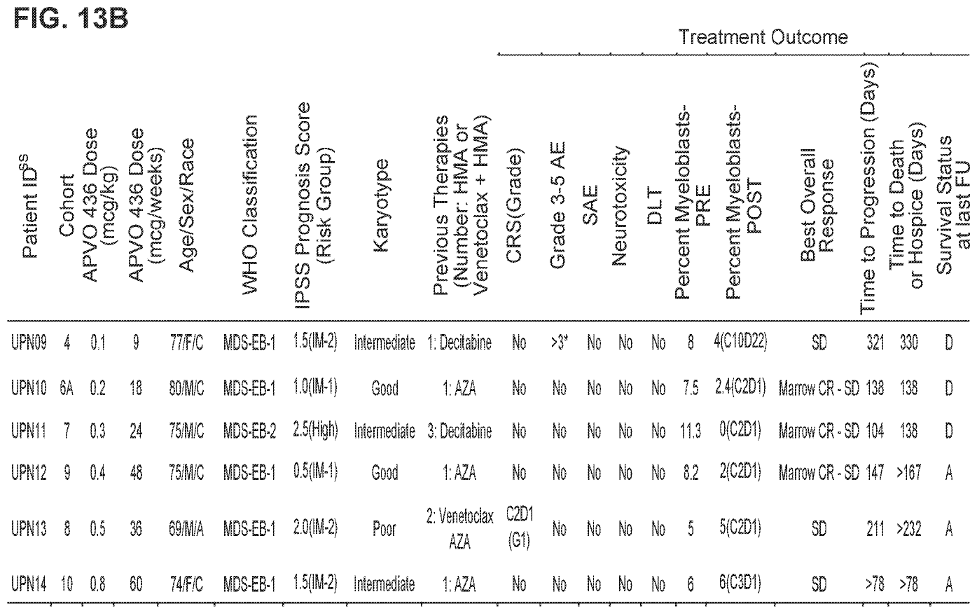

Patient characteristics: Eight patients, including 3 males and 5 females with a median age of 66 years (Mean±SE=65±6 years) of whom 7 were Caucasian and 1 was African-American, had R/R AML with 1-8 lines of prior AML therapy (FIG. 13A). 4 males and 2 females had R/R MDS. They had a median age of 75 years (Mean±SE=75±2 years) of whom 5 were Caucasian and 1 was Asian, had R/R MDS with 1-3 lines of prior MDS therapy (FIG. 13B). Of the 8 AML patients, 4 (UPN01, UPN02, UPN04, UPN06) had AML with MDS-related features—one of these patients (UPN04) also had FLT3-ITD gene mutation, 1 (UPN03) had AML with recurrent genetic abnormalities, 2 had AML with gene mutations (UPN07, UPN08), and 1 had AML-NOS (MO-AML) (FIG. 13A). All 6 MDS patients had MDS with excess blasts according to WHO classification (MDS-EB-1 or MDS-EB-2). Five of these patients had IPSS prognosis scores consistent with an intermediate-1 (IM-1) or intermediate-2 (IM-2) risk group and one had high-risk MDS (FIG. 13B). Patient characteristics and treatment outcome data are shown in FIG. 13A-13B.

No dose-limiting toxicities (DLT) or Grade 5 adverse events (AE) were observed in any of the 14 cases analyzed. Among the 8 AML patients, 6 (UPN01, UPN02, UPN03, UPN04, UPN-7, and UPN08) had no CRS, one patient (UPN05) had Grade 1 CRS lasting 2 days, and one patient (UPN06) had transient Grade 2 CRS lasting 2 days (FIG. 13A-13B). There were 2 severe adverse events (SAEs): One SAE was reported for UPN05 who developed sepsis, diarrhea and vomiting on C6D5 showing full recovery within 5 days. Grade 2 CRS of UPN06 was also reported as an SAE due to hospitalization but lasted only 2 days and fully resolved (FIG. 13A-13B). No SAEs were reported for any of the 6 MDS patients. One MDS patient (UPN13) experienced Grade 1 CRS on C2D1 lasting one day and another MDS patient (UPN09) experienced transient several Grade 3-4 AEs, including tumor lysis syndrome (TLS), Anemia, decreased platelet count, and hyperglycemia (FIG. 13A-13B).

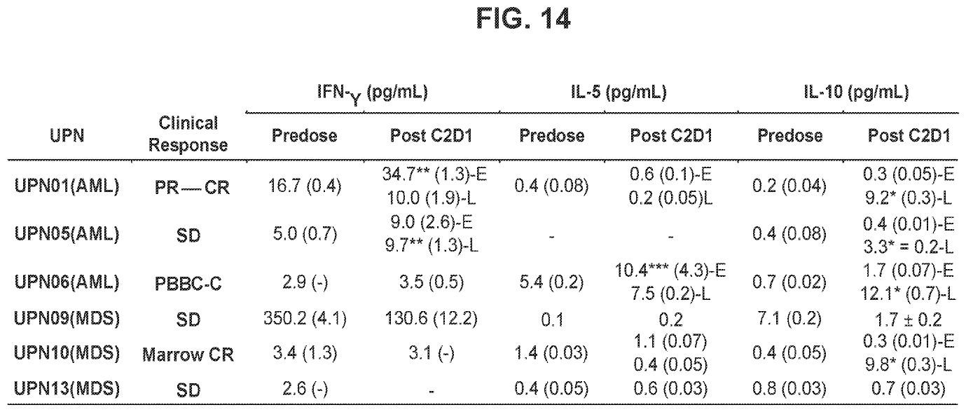

Pharmacodynamic Effects of TRI130: An analysis was performed to determine if TRI130 can activate T-cells by measuring serum levels of three T-cell derived cytokines (IFN-γ, IL-5 and IL-10) as surrogate pharmacodynamic biomarkers: T-helper 1 cytokines: IFN-γ, IL-10; T-helper 2 cytokines: IL-5, IL-10; effector T cell cytokine: IL-10). Serum levels of IFN-γ and IL-10 were markedly elevated above baseline in post-treatment blood samples from R/R AML patients (UPN01, UPN05 and UPN06) consistent with treatment-induced activation of T-cells. Likewise, IL-10 level was above baseline in a post-treatment sample from one MDS patient UPN10 (but not MDS patients UPN09 or UPN13) (FIG. 14).

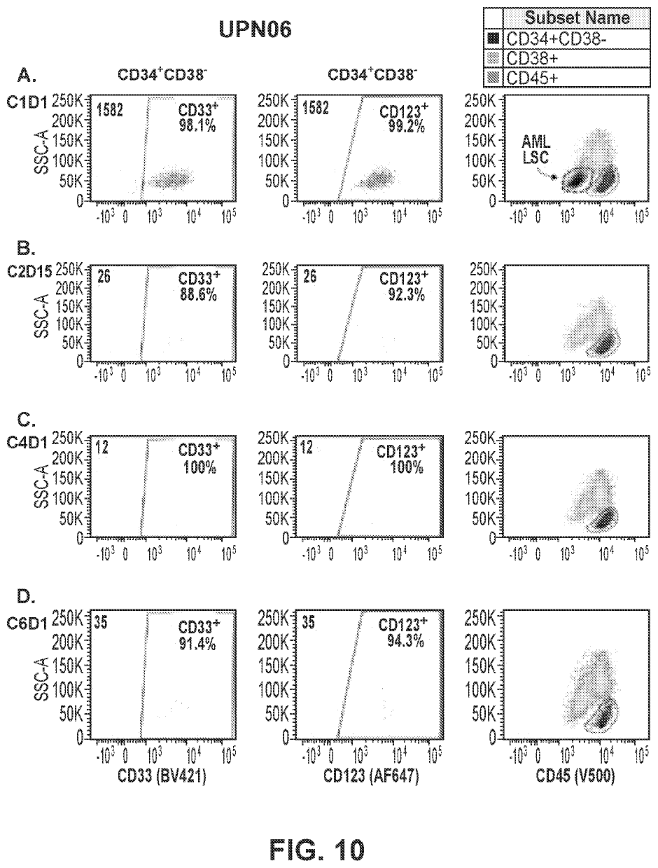

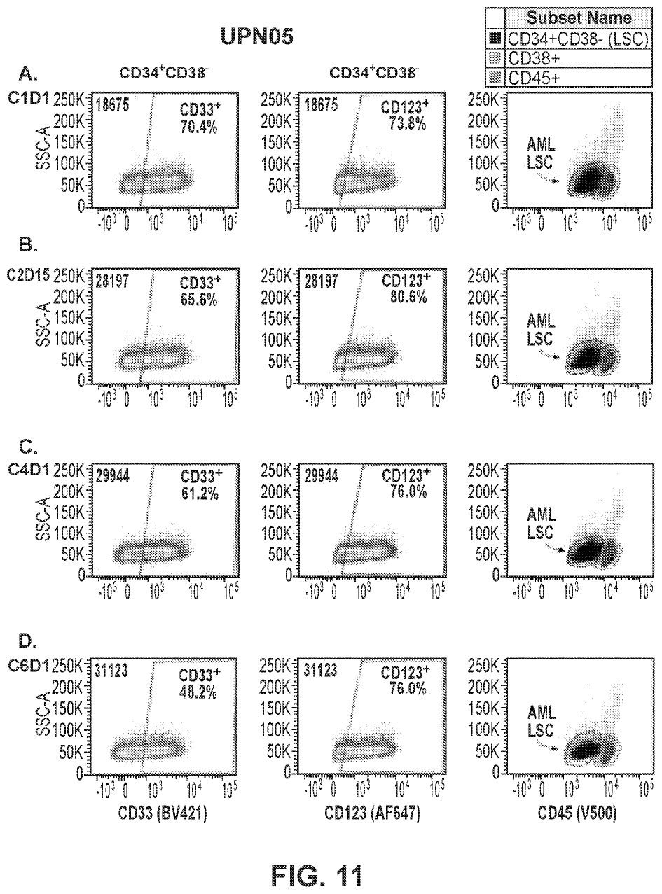

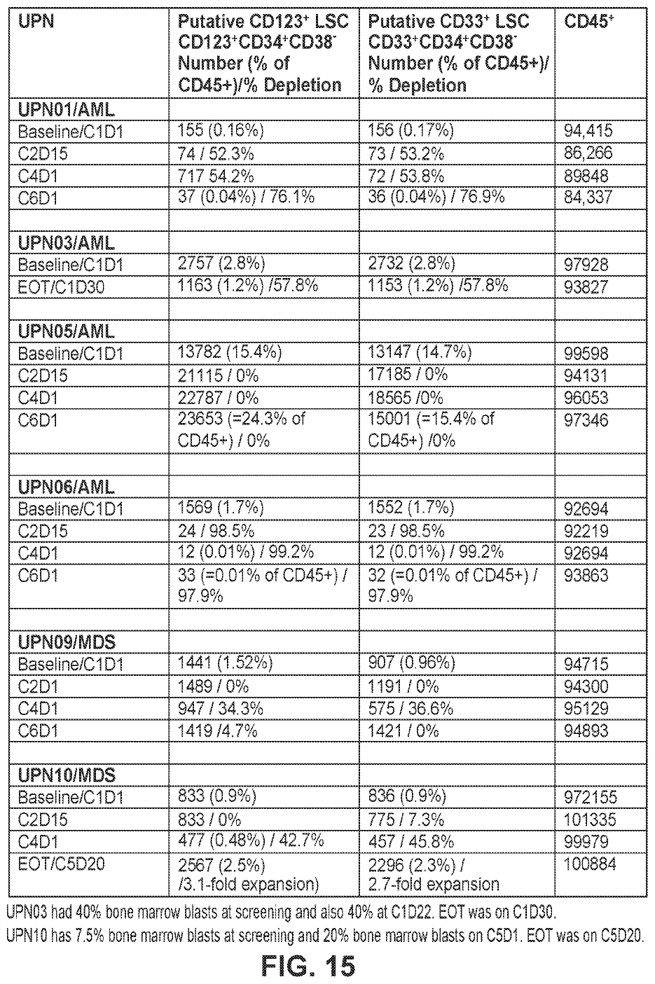

Next, immunophenotyping by multiparameter flow cytometry was used to examine the effects of TRI130 on tumor burden reflected by CD123+ target blast cells. Unlike the normal CD34+CD38− hematopoietic stem cells, the putative leukemic stem cell (LSC) populations in AML have been reported to express CD123 (19). Most of these cells co-express CD33 antigen (i.e., they are CD33+CD34+CD38−). Flow cytometric analysis of post-treatment blood samples from 3 of 4 AML patients (UPN01, UPN03, UPN06) showed decreased numbers of the circulating CD123+CD34+CD38− and CD33+CD34+CD38− putative LSC populations (FIG. 15). The maximum reduction in the numbers of the CD123+ LSC population were 76.1% in UPN01 (C6D1 sample), 57.8% in UPN03 (C1 D30 sample), and 99.2% in UPN06 (C4D1 sample). FIG. 10 depicts the multicolor dot profiles from the multi-parameter flow cytometry test that illustrates the rapid and marked depletion of the CD123+CD34+CD38− and CD33+CD34+CD38− LSC populations by TRI130 monotherapy in UPN06. Virtually all of the CD34+CD38− cells were CD123+ and CD33+ consistent with AML. The size of this CD123+CD33+CD34+CD38− AML LSC population indicated with the arrow in Panel A, 3rd column, was significantly reduced by TRI130 monotherapy. In contrast to these 3 AML cases, FIG. 15 and FIG. 11 illustrate that the CD34+CD38-CD123+ cells from the 4th AML patient, UPN05, were not depleted to TRI130 treatments, but their expansion was prevented which was associated with stable disease with a time to progression of 238 days. As shown in FIG. 15, reductions of CD123+CD34+CD38− and CD33+CD34+CD38− cells were also observed in post-treatment samples of two MDS patients.

These pharmacodynamic results provide early proof of concept that the CD3×CD123 bispecific TRI130 is capable of activating T-cells and causing depletion of targeted CD123+ blast cells in patients with MDS and AML. Notably, at the time of progression, there was a significant expansion of the CD123+ population in UPN10 and the cells in UPN03 that were not depleted were CD123+. These results illustrate that TRI130 monotherapy failures can occur independent of CD123 expression as the anti-leukemic activity of TRI130 is dependent on the cytotoxic T-cell (CTL) activity of the CD3-expressing T-cell populations that are redirected to CD123+ AML/MDS cells.

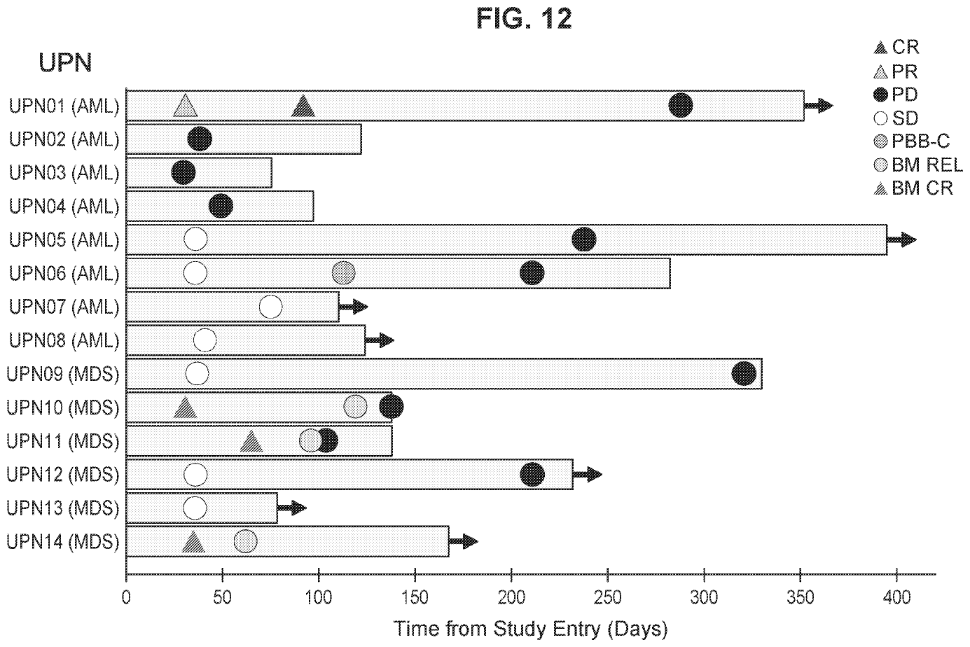

Efficacy: Of the 8 R/R AML patients, 2 patients (UPN2 and UPN4) had progressive disease (PD) and one had a stable disease (SD)/resistant disease (RES) (UPN3) as best overall response (BOR) and died of leukemia between 75-122 days (FIG. 13A-13B). Three patients (including UPN01, UPN05 and UPN06) had prolonged SD. Their time to progression ranged from >106 days to 238 days. Of 3 AML patients with a SD as their best overall response, one patient (UPN06; AML with MDS related features) had a complete clearance of peripheral blasts at 113 days (from 21% pre-treatment to 14% in cycle 2, 2% in cycle 3, and 0% in cycle 4) and a >50% decrease of pretreatment bone marrow blast percentage (from 78% prior to treatment to 37% after treatment), followed by sustained SD. This patient also had evidence of pharmacodynamic activation of T-cells by TRI130 (FIG. 14) and depletion of target CD123+CD34+CD38− AML cells (FIG. 15). Another AML patient with MDS-related features (UPN01), who had 29% bone marrow blasts, unfavorable cytogenetics (del 5q and monosomy 7) and TP53 mutation, whose disease had previously progressed on Venetoclax+Decitabine therapy, achieved a PR at 31 days and CR at 92 days following TRI130 monotherapy with full hematologic recovery as BOR (best overall response). This patient also had evidence of pharmacodynamic activation of T-cells by TRI130 (FIG. 14) and depletion of target CD123+CD34+CD38− blast cells (FIG. 15). The onset and duration of the SD, peripheral blood blast count clearance (PBBC-C), PR or CR in these patients is illustrated by the Swimmer plot depicted in FIG. 12.

Likewise, 3 MDS patients had SD (50%) and 3 additional MDS patients (50%) had a bone marrow CR at dose levels ranging from 0.1 mcg/kg to 0.8 mcg/kg (FIG. 13A-13B). One patient (UPN10 from Cohort 9) whose posttreatment samples had shown activation of T-cells (FIG. 14) had a pretreatment BM blasts of 8.2% with 15% marrow cellularity and a C2D1 posttreatment BM showing 2% blasts with 20% cellularity. Another patient (2000003 from Cohort 7) had baseline BM blasts of 11.3% with 20-30% cellularity and a C2D1 posttreatment BM blast percentage of 0% with 50% cellularity. The third patient (2190001 from Cohort 6A) had a pretreatment BM blast percentage of 7.5% with 10% cellularity and a C2D1 posttreatment BM blast percentage of 2.4% with 20% cellularity (FIG. 13A-13B). The onset of marrow CR and time to progression in these MDS patients is illustrated by the Swimmer plot depicted in FIG. 12. One MDS patient, UPN09 in Cohort 4, with ASXL-1 mutation showed a decrease in the percentage of myeloblasts in the bone marrow, but this was part of the transformation of the case into a high-risk chronic myelomonocytic leukemia-myeloproliferative neoplasm (CMMN-MPN) with an absolute monocyte count 17,600/μL around Cycle 10 (FIG. 13A-13B).

Summary: The data presented herein shows that TRI130 activated T-cells in R/R AML and MDS patients as evidenced by marked elevation of serum Th1/Th2 cytokine IL-10 and reduced the numbers of circulating CD123+CD34+ and CD33+CD34+ peripheral blasts. Single agent activity was observed at dose levels ranging from 0.1 μg/kg to 0.7 μg/kg in 4 R/R AML patients (50%), including 3 patients with prolonged stable disease (SD) and one patient with complete remission (CR). Likewise, 3 MDS patients had SD (50%) and 3 additional MDS patients (50%) had a marrow CR at dose levels ranging from 0.1 μg/kg to 0.8 μg/kg. These data provide proof of concept supporting the in vivo immunomodulatory and antileukemic activity of TRI130.

This analysis also provides clinical proof of concept evidence supporting the mechanism of action of TRI130 using the primary data from a recently completed Phase 1B study in R/R AML and MDS patients [28]. These results informed the design of currently accruing Cohort 2 of the Expansion phase of the Phase 1B study (NCT03647800). This data demonstrates promising early single agent activity and immunomodulatory effects of TRI130 (given as a weekly IV infusion) in patients who have failed prior treatment with Venetoclax plus HMA for AML or HMA alone for MDS. Although single agent TRI130 has not been associated with dose-limiting myelosuppression, the tolerability and efficacy of TRI130 in combination with a Venetoclax plus Azacitidine backbone will be formally evaluated in a Phase IB study in newly diagnosed AML patients≥75 years of age or those who are over 60 years of age and unfit for intensive chemotherapy or HSCT (Clinicaltial.gov identifier: NCT04973618). There will be a 2-week lead-in phase of Venetoclax plus Azacitidine to mitigate risk of TLS and cytokine release syndrome (CRS). It is hypothesized that the addition of TRI130 will eradicate residual CD123+ blasts as well as leukemic stem cells that are resistant to Venetoclax, leading to more durable responses.

Therapy-naive patients with AML who are elderly (>=75) or unfit to receive intensive chemotherapy have improved survival when treated with Venetoclax and HMA compared to HMA alone [7,31], and the Food and Drug Administration (FDA) has approved Venetoclax in combination with HMA for this population [15]. However, with increasing clinical experience involving the use of Venetoclax, several challenges and limitations of Venetoclax-based therapy have emerged, as emphasized in recent publications [13, 32].

Venetoclax-induced remissions in therapy-naive older/unfit AML patients are short-lived lasting less than 12 months (median) even after combined use of Venetoclax and HMAs. Furthermore, patients with secondary AML as well as AML patients previously treated with HMAs are less responsive to Venetoclax-based treatment regimens with substantially worse CR rates and <6 month overall survival times [13]. Likewise, some AML patient populations in the adverse risk category, such as patients with TP53 mutations and RTK mutations, may exhibit inherent Venetoclax-resistance [13]. Unlike its remarkable activity in therapy-naive AML patients, Venetoclax is not very effective in relapsed AML patients. The reported overall response rate was only 21% for relapsed or refractory AML patients treated with venetoclax in combination with HMAs, LDAC, or other agents such as cladribine or midostaurin [33]. Therefore, new agents that can potentially be combined with and improve the clinical efficacy of Venetoclax-based treatment regimens for elderly AML patients are urgently needed.

### REFERENCES

1. Ferrara, F.; Lessi, F.; Vitagliano, O.; Birkenghi, E.; Rossi, G. Cancers (Basel). 2019; 11(2). pii: E224. doi: 10.3390/cancers11020224.
2. DiNardo C D, Wei A H. Blood (2020) 135(2):85-96. 10.1182/blood.2019001239.
3. Blum W G, Mims A S. Cancer 2020; 126(21): 4668-4677. https://doi.org/10.1002/cncr.32904.
4. Short, N.J., Konopleva, M., Kadia, T. M., Borthakur, G., Ravandi, F., DiNardo, C. D., Daver, N. Cancer Discov. 2020; 10(4):506-525. doi: 10.1158/2159-8290.CD-19-1011.
5. Daver, N., Wei, A. H., Pollyea, D. A, Fath, i A. T., Vyas, P., DiNardo, C. D. Blood Cancer J 2020; 10:107. 10.1038/s41408-020-00376-1.
6. Dohner, H.; Estey, E.; Grimwade, D.; Amadori, S.; Appelbaum, F. R.; Buchner, T.; Dombret, H.; Ebert, B. L.; Fenaux, P.; Larson, R. A.; Levine, R. L.; Lo-Coco, F.; Naoe, T.; Niederwieser, D.; Ossenkoppele, G. J.; Sanz, M.; Sierra, J.; Tallman, M. S.; Tien, H. F.; Wei, A. H.; Lowenberg, B.; Bloomfield, C. D. Blood 2017, 129, 424-447.
7. DiNardo, C. D.; Pratz, K.; Pullarkat, V.; Jonas, B. A.; Arellano, M.; Becker, P. S.; Frankfurt, O.; Konopleva, M.; Wei, A. H.; Kantarjian, H. M.; et al. Blood. 2019, 133, 7-17. doi: 10.1182/blood-2018-08-868752.
8. Othman T A, Azenkot T, Moskoff B N, Tenold M E, Jonas B A. Future Oncol. 2021 May 24. doi: 10.2217/fon-2021-0262.
9. DiNardo C D, Jonas B A, Pullarkat V, Thirman M J, Garcia J S, Wei A H, et al. New Engl J Med (2020) 383(7):617-29. 10.1056/NEJMoa2012971.
10. Mims, A. S.; Blum, W. Curr Opin Hematol. 2019; 26(2), 88-95. doi: 10.1097/MOH.0000000000000490.
11. Schlenk, R. F.; Muller-Tidow, C.; Benner, A. Curr Opin Oncol 2017, 29, 467-473.
12. Kapoor, I., Bodo, J., Hill, B. T. et al. Cell Death Dis 11, 941 (2020). https://doi.org/10.1038/s41419-020-03144-y.
13. Pollyea, D. A., Stevens, B. M., Jones, C. L., Winters, A., Pei, S., Minhajuddin, M., D'Alessandro, A., Culp-Hill, R., Riemondy, K. A., Gillen, A. E., et al. (2018). Nat. Med. https://doi.org/10.1038/s41591-018-0233-1.
14. Lagadinou E D, Sach A, Callahan K, Rossi R M, Neering S J, Minhajuddin M, Ashton J M, Pei S, Grose V, O'Dwyer K M, Liesveld J L, Brookes P S, Becker M W, Jordan C T. Cell Stem Cell. 2013 Mar. 7:12(3):329-41. doi: 10.1016/j.stem.2012.12.013. Epub 2013 Jan. 17.
15. Jones C L, Stevens B M, D'Alessandro A, Reisz J A, Culp-Hill R, Nemkov T, Pei S, Khan N, Adane B, Ye H, Krug A, Reinhold D, Smith C, DeGregori J, Pollyea D A, Jordan C T. Cancer Cell. 2018 Nov. 12; 34(5):724-740.e4. doi: 10.1016/j.ccell.2018.10.005. Erratum in: Cancer Cell. 2019 Feb. 11; 35(2):333-335.
16. Testa, U., Riccion, i R., Coccia, E., Stellacci, E., Samoggia, P., Latagliata, R., Latagliata, R., Mariani, G., Rossini, A., Battistini, A., Lo-Coco, F., Peschle, C. Blood 2002; 100(8):2980-2988.
17. Hwang, K., Park, C. J., Jang, S., Chi, H. S., Kim, D. Y., Lee, J. H., Im, H. J., Seo, J. J. Ann Hematol 2012; 91(10):1541-1546.
18. Jin, L., Lee, E. M., Ramshaw, H. S., Busfiled, S. J., Peoppl, A. G., Wilkinson, L., Wilkinson, L., Guthridge, M. A., Thomas, D., Barry, E. F., Boyd, A., Gearing, D. P, Vairo, G., Lopez, A. F., Dick, J. E., Lock, R. B. Cell Stem Cell 2009; 5(1):31-42.
19. Al-Mawali A, Pinto A D, Al-Zadjali S. Acta Haematol. 2017; 138(3):175-181, doi: 10.1159/000480448.
20. Vergez F, Green A S, Tamburini J, et al. Haematologica. 2011; 96(12):1792-1798.
21. A L Hussaini M H, Ritchey J, Rettig M P, Eissenberg L, Uy G L, Chichili G, et al. Blood (2013) 122:360-0. 10.1182/blood.V122.21.360.360.
22. Uy G L, Aldoss I, Foster M C, Sayre P H, Wieduwilt M J, Advani A S, et al. Blood (2021) 137(6):751-62. 10.1182/blood.2020007732.
23. Daver N, Alotaibi A S, Bucklein V, Subklewe M. Leukemia. 2021 May 5. doi:10.1038/s41375-021-01253-x.
24. Isidori A, Cerchione C, Daver N, DiNardo C, Garcia-Manero G, Konopleva M, Jabbour E, Ravandi F, Kadia T, Burguera A F, Romano A, Loscocco F, Visani G, Martinelli G, Kantarjian H, Curti A. Front Oncol. 2021 May 10; 11:656218. doi: 10.3389/fonc.2021.656218.
25. Comeau, M. R.; Gottschalk, R.; Daugherty, M.; Sewell, T.; Sewell, T.; Misher, L.; Bannink, J.; Johnson, S.; Parr, L.; Kumer, J.; Jablonski, D.; DeFrancesco, M.; Bienvenue, D.; Hoyos, G. H.; McMahan, C. J.; Gross, J. A. [abstract]. In: Proceedings of the American Association for Cancer Research Annual Meeting 2019; 2019 Mar. 29-Apr. 3; Atlanta, GA. Philadelphia (PA): AACR; Cancer Res 2019; 79(13 Suppl): Abstract nr LB-199.
26. Comeau, M. R.; Miller, R. E.; Bannink, J.; Johnson, S.; Bader, R.; Gottschalk, R.; Misher, L.; Mitchell, D.; Parr, L.; DeFrancesco, M.; Bienvenue, D.; McMahan, C. J.; Hoyos, G. H.; Gross, J. A. [abstract]. In: Proceedings of the AACR-NCI-EORTC International Conference: Molecular Targets and Cancer Therapeutics; 2017 Oct. 26-30; Philadelphia, PA. Philadelphia (PA): AACR; Mol Cancer Ther 2018; 17(1 Suppl):Abstract nr B111.
27. Comeau, M. R.; Miller, R. E.; Bannink, J.; Johnson, S.; Bader, R.; Gottschalk, R.; Daugherty, M.; Sewell, T.; Misher, L.; Mitchell, D.; Parr, L.; DeFrancesco, M.; Bienvenue, D.; McMahan, C. J.; Hoyos, G. H.; Gross, J. A. [abstract]. In: Proceedings of the American Association for Cancer Research Annual Meeting 2018; 2018 Apr. 14-18; Chicago, IL. Philadelphia (PA): AACR; Cancer Res 2018; 78(13 Suppl):Abstract nr 1786.
28. Uckun, F. M.; Lin, T. L.; Mims, A.; Patel, P.; Lee, C.; Shahidzadeh, A.; Shami, P.; Cull, E.; Cogle, C. R.; Watts, J. Cancers 2021, 13, Aug. 15; 13(16):4113. doi: 10.3390/cancers13164113.
29. Uckun F M, Cogle C R, Lin T L, Qazi S, Trieu V N, Schiller G, Watts J M. Cancers (Basel). 2019 Dec. 26; 12(1):74. doi: 10.3390/cancers12010074.
30. Uckun F M, Qazi S, Hwang L, Trieu V N. Cancers (Basel). 2019 Nov. 28; 11(12):1892. doi: 10.3390/cancers11121892.
31. DiNardo C D, Pratz K W, Letai A, et al. Lancet Oncol. 2018; 19(2):216-228
32. Feld, J., Tremblay, D., Dougherty, M., Czaplinska, T., Sanchez, G., Brady, C., Kremyanskaya, M., Bar-Natan, M., Keyzner, A., Marcellino, B. K., Gabrilove, J., Navada, S. C., Silverman, L. R., El Jamal, S. M., Mascarenhas, J., & Shih, A. H. (2021). HemaSphere, 5(4), e549. https://doi.org/10.1097/HS9.0000000000000549.
33. DiNardo C D, Rausch C R, Benton C, et al. Am J Hematol. 2018; 93(3):401-407.
34. Lee J B, Khan D H, Hurren R, Xu M, Na Y, Kang H, Mirali S, Wang X, Gronda M, Jitkova Y, MacLean N, Arruda A, Alaniz Z, Konopleva M Y, Andreeff M, Minden M D, Zhang L, Schimmer A D. Blood. 2021 Jul. 22; 138(3):234-245. doi: 10.1182/blood.2020009081.

1. A method for treating a cancer, the method comprising administering to a subject in need thereof:
i) a multispecific protein comprising a CD123 binding domain and a CD3 binding domain; and
ii) a second anti-cancer agent.

2. The method of claim 1, wherein the method further comprises administering to the subject a third anti-cancer agent.

3. The method of claim 1 or 2, wherein the second anti-cancer agent is a chemotherapeutic drug.

4. The method of claim 3, wherein the chemotherapeutic drug is venetoclax, azacitidine, decitabine, daunorubicin, cytarabine, idarubicin, mitoxantrone, or etoposide.

5. The method of claim 1, wherein the second anti-cancer agent is cytarabine.

6. The method of claim 5, wherein the cytarabine is administered at a dose of about 1 g/m².

7. The method of claim 2, wherein the second anti-cancer agent is mitoxantrone, and the third anti-cancer agent is etoposide, and wherein the method further comprises administering a fourth anti-cancer agent that is cytarabine.

8. The method of claim 7, wherein the mitoxantrone is administered at a dose of about 6 mg/m²/day, the etoposide is administered at a dose of about 80 mg/m²/day, and the cytarabine is administered at a dose of about 1 g/m²/day.

9. The method of claim 1, wherein the second anti-cancer agent is venetoclax.

10. The method of claim 9, wherein the venetoclax is administered at a dose of from about 100 to about 400 mg per day.

11. The method of claim 1, wherein the second anti-cancer agent is azacytidine.

12. The method of claim 11, wherein the azacytidine is administered at a dose of about 75 mg/m²/day.

13. The method of claim 2, wherein the second anti-cancer agent is azacitidine and the third anti-cancer agent is venetoclax.

14. The method of claim 13, wherein the azacitidine is administered at a dose of about 75 mg/m²/day and the venetoclax is administered at a dose of from about 100 to about 400 mg/day.

15. The method of claim 1, wherein the second anti-cancer agent is decitabine.

16. The method of claim 15, wherein the decitabine is administered at a dose of about 20 mg/m²/day.

17. The method of claim 2, wherein the second anti-cancer agent is decitabine and the third anti-cancer agent is venetoclax.

18. The method of claim 17, wherein the decitabine is administered at a dose of about 20 mg/m²/day and the venetoclax is administered at a dose of from about 100 to about 400 mg/day.

19. The method of claim 2, wherein the second anti-cancer agent is daunorubicin, and the third anti-cancer agent is cytarabine.

20. The method of claim 19, wherein the daunorubicin is administered at a dose of about 30-90 mg/m² and the cytarabine is administered at a dose of from about 100 to about 200 mg/m².

21. The method of claim 1, wherein the anti-cancer agent is idarubicin.

22. The method of claim 21, wherein the idarubicin is administered at a dose of about 12 mg/m².

23. The method of claim 2, wherein the second anti-cancer agent is idarubicin and the third anti-cancer agent is cytarabine.

24. The method of claim 23, wherein the idarubicin is administered at a dose of about 12 mg/m², and cytarabine is administered at a dose of from about 100 to about 200 mg/m².

25. The method of claim 1, wherein the second anti-cancer agent is azacitidine.

26. The method of claim 5, wherein the azacitidine is administered at a dose of about 75 mg/m²/day.

27. The method of any one of claims 1-26, wherein the multispecific protein comprises: a dimer of two identical polypeptides, wherein each polypeptide comprises, in order from amino-terminus to carboxyl-terminus, or in order from carboxyl-terminus to amino-terminus:
(i) a CD123 binding domain,
(ii) a hinge region,
(iii) an immunoglobulin constant region, and
(iv) a CD3 binding domain.

28. The method of claim 27, wherein the polypeptide comprises, from N-terminus to C-terminus, the CD123 binding domain, the hinge region, the immunoglobulin constant region, and the CD3 binding domain.

29. The method of claim 28, wherein at least one of the CD123 and the CD3 binding domains comprises:
(i) an immunoglobulin heavy chain variable region (V_{H}) comprising HCDR1, HCDR2, and HCDR3; and
(ii) an immunoglobulin light chain variable region (V_{L}) comprising LCDR1, LCDR2, and LCDR3.

30. The method of claim 29, wherein the CD123 binding domain is a scFv comprising:
a HCDR1 that comprises SEQ ID NO: 10, a HCDR2 that comprises SEQ ID NO: 11, and a HDCR3 that comprises SEQ ID NO: 12; and
a LCDR1 that comprises SEQ ID NO: 13, a LCDR2 that comprises SEQ ID NO: 14, and a LCDR3 that comprises SEQ ID NO: 15.

31. The method of claim 29, wherein the CD123 binding domain is a scFv comprising:
a V_{H} comprising a sequence at least 90%, at least 95%, or 100% identical to SEQ ID NO: 136, and
a V_{L} comprising a sequence at least 90%, at least 95%, or 100% identical to SEQ ID NO: 134.

32. The method of claim 29, wherein the CD123 binding domain is a scFv, and wherein the scFv comprises a sequence at least 90%, at least 95%, or 100% identical to SEQ ID NO: 27.

33. The method of any one of claims 29-32, wherein the CD3 binding domain is a scFv comprising:
a HCDR1 that comprises SEQ ID NO: 19, a HCDR2 that comprises SEQ ID NO: 20, and a HDCR3 that comprises SEQ ID NO: 21; and
a LCDR1 that comprises SEQ ID NO: 22, a LCDR2 that comprises SEQ ID NO: 23, and a LCDR3 that comprises SEQ ID NO: 24.

34. The method of any one of claims 29-32, wherein the CD3 binding domain is a scFv that comprises:
a V_{H} comprising a sequence at least 90%, at least 95%, or 100% identical to SEQ ID NO: 383 or 387, and
a V_{L} comprising a sequence at least 90%, at least 95%, or 100% identical to SEQ ID NO: 384.

35. The method of any one of claims 29-32, wherein the CD3 binding domain is a scFv that comprises a sequence at least 90%, at least 95%, or 100% identical to SEQ ID NO: 27.

36. The method of claim 29, wherein each polypeptide comprises a sequence at least 90%, at least 95%, or 100% identical to SEQ ID NO: 31.

37. The method of any one of claims 1-36, wherein the multispecific protein is administered to the subject by IV infusion.

38. The method of any one of claims 1-37, wherein the second anti-cancer agent is administered to the subject orally or by IV infusion.

39. The method of any one of claims 1-38, wherein the multispecific protein is administered the subject by IV infusion at a dose of 0.3, 1, 3, 6, 9, 12, 18, 20, 24, 30, 36, 48, 50, 60, 75, or 100 μg.

40. The method of any one of claims 1-39, wherein the multispecific protein is administered once per week.

41. The method of claim 40, wherein the multispecific protein is administered to the subject by IV infusion during a first 28-day cycle; wherein 6 μg of the multispecific protein is administered on day 8; 12 μg of the multispecific protein is administered on day 15; and 18 μg of the multispecific protein is administered on day 22 of the first 28-day cycle.

42. The method of claim 41, wherein the multispecific protein is administered to the subject by IV infusion during at least one additional 28-day cycle following the first 28-day cycle, wherein 18 μg of the multispecific protein is administered on days 1, 8, 15, and 22 of the at least one additional 28-day cycle.

43. The method of claim 42, wherein cytarabine is administered intravenously on days 1-5 of the first 28-day cycle, and days 1-5 of at least one additional 28-day cycle.

44. The method of claim 43, wherein the dose of cytarabine is about 1 g/m².

45. The method of claim 42, wherein mitoxantrone, etoposide, and cytarabine are administered intravenously on days 1-6 of the first 28-day cycle, and at least one additional 28-day cycle.

46. The method of claim 45, wherein the dose of mitoxantrone is about 6 mg/m²/day, the dose of etoposide is about 80 mg/m²/day, and the dose of cytarabine is about 1 g/m²/day.

47. The method of claim 40, wherein the multispecific protein is administered to the subject by IV infusion during a first 28-day cycle; wherein 6 μg of the multispecific protein is administered on day 15; and 12 μg of the multispecific protein is administered on day 22 of the first 28-day cycle.

48. The method of claim 47, wherein the multispecific protein is administered to the subject by IV infusion during at least one additional 28-day cycle following the first 28-day cycle, wherein 18 μg of the multispecific protein is administered on days 1, 8, 15, and 22 of the at least one additional 28-day cycle.

49. The method of claim 48, wherein venetoclax is administered orally on days 1-21 of the first 28-day cycle, and days 1-21 of at least one additional 28-day cycle.

50. The method of claim 49, wherein the dose of venetoclax is from about 100 to about 400 mg/day.

51. The method of any one of claims 48-50, wherein azacytidine is administered intravenously on days 1-7 of the first 28-day cycle, and at least one additional 28-day cycle.

52. The method of claim 51, wherein the dose of azacytidine is about 75 mg/m².

53. The method of claim 40, wherein the multispecific protein is administered to the subject by IV infusion during a first 28-day cycle, wherein 6 μg of the multispecific protein is administered on day 1; 8 μg of the multispecific protein is administered on day 12; 18 μg of the multispecific protein is administered on day 15; and 18 μg of the multispecific protein is administered on day 22 of the first 28-day cycle.

54. The method of claim 53, wherein the multispecific protein is administered to the subject by IV infusion during at least one additional 28-day cycle following the first 28-day cycle, wherein 18 μg of the multispecific protein is administered on days 1, 8, 15, and 22.

55. The method of claim 54, wherein cytarabine is administered by intravenous infusion on days 1-7 of the first 28-day cycle, and days 1-7 of at least one additional 28-day cycle.

56. The method of claim 55, wherein the dose of cytarabine is from about 100 to about 200 mg/m².

57. The method of any one of claims 53-56, wherein idarubicin is administered by intravenous infusion on days 1-3 of the first 28-day cycle, and days 1-3 of at least one additional 28-day cycle.

58. The method of claim 57, wherein the dose of idarubicin is about 12 mg/m².

59. The method of claim 40, wherein the multispecific protein is administered to the subject by IV infusion during a first 28-day cycle, wherein 6 μg of the multispecific protein is administered on day 1; 8 μg of the multispecific protein is administered on day 12; 18 μg of the multispecific protein is administered on day 15; and 18 μg of the multispecific protein is administered on day 22 of the first 28-day cycle.

60. The method of claim 59, wherein the multispecific protein is administered to the subject by IV infusion during at least one additional 28-day cycle following the first 28-day cycle, wherein 18 μg of the multispecific protein is administered on days 1, 8, 15, and 22 of the at least one additional 28-day cycle.

61. The method of claim 60, wherein azacytidine is administered orally on days 1-14 of the first 28-day cycle, and at least one additional 28-day cycle.

62. The method of claim 61, wherein the dose of azacytidine is about 300 mg/day.

63. The method of any one of claims 1-62, wherein the cancer is a carcinoma or sarcoma.

64. The method of any one of claims 1-62, wherein the cancer is melanoma, kidney cancer, pancreatic cancer, lung cancer, intestinal cancer, prostate cancer, breast cancer, liver cancer, brain cancer, colon cancer, ovarian cancer, or hematological cancer.

65. The method of any one of claims 1-62, wherein the cancer is acute myeloid leukemia (AML), myelodysplastic syndrome (MDS), hairy cell leukemia (HCL), blastic plasmacytoid dendritic cell neoplasm, B-cell acute lymphoblastic leukemia (ALL), or chronic myeloid leukemia (CML).

66. The method of any one of claims 1-62, wherein the cancer is acute myeloid leukemia (AML).

67. The method of any one of claims 1-62, wherein the cancer is myelodysplastic syndrome (MDS).

68. A method for treating a cancer, the method comprising administering to a subject in need thereof a multispecific protein comprising a CD123 binding domain and a CD3 binding domain.

69. The method of claim 68, wherein the multispecific protein comprises: a dimer of two identical polypeptides, wherein each polypeptide comprises, in order from amino-terminus to carboxyl-terminus, or in order from carboxyl-terminus to amino-terminus:
(i) a CD123 binding domain,
(ii) a hinge region,
(iii) an immunoglobulin constant region, and
(iv) a CD3 binding domain.

70. The method of claim 69, wherein the polypeptide comprises, from N-terminus to C-terminus, the CD123 binding domain, the hinge region, the immunoglobulin constant region, and the CD3 binding domain.

71. The method of claim 69, wherein at least one of the CD123 and the CD3 binding domains comprises:
(i) an immunoglobulin heavy chain variable region (VH) comprising HCDR1, HCDR2, and HCDR3; and
(ii) an immunoglobulin light chain variable region (VL) comprising LCDR1, LCDR2, and LCDR3.

72. The method of claim 69, wherein the CD123 binding domain is a scFv comprising:
a HCDR1 that comprises SEQ ID NO: 10, a HCDR2 that comprises SEQ ID NO: 11, and a HDCR3 that comprises SEQ ID NO: 12; and
a LCDR1 that comprises SEQ ID NO: 13, a LCDR2 that comprises SEQ ID NO: 14, and a LCDR3 that comprises SEQ ID NO: 15.

73. The method of claim 69, wherein the CD123 binding domain is a scFv comprising:
a VH comprising a sequence at least 90%, at least 95%, or 100% identical to SEQ ID NO: 136, and
a VL comprising a sequence at least 90%, at least 95%, or 100% identical to SEQ ID NO: 134.

74. The method of claim 69, wherein the CD123 binding domain is a scFv, and wherein the scFv comprises a sequence at least 90%, at least 95%, or 100% identical to SEQ ID NO: 27.

75. The method of any one of claims 69-74, wherein the CD3 binding domain is a scFv comprising:
a HCDR1 that comprises SEQ ID NO: 19, a HCDR2 that comprises SEQ ID NO: 20, and a HDCR3 that comprises SEQ ID NO: 21; and
a LCDR1 that comprises SEQ ID NO: 22, a LCDR2 that comprises SEQ ID NO: 23, and a LCDR3 that comprises SEQ ID NO: 24.

76. The method of any one of claims 69-74, wherein the CD3 binding domain is a scFv that comprises:
a VH comprising a sequence at least 90%, at least 95%, or 100% identical to SEQ ID NO: 383 or 387, and
a VL comprising a sequence at least 90%, at least 95%, or 100% identical to SEQ ID NO: 384.

77. The method of any one of claims 69-74, wherein the CD3 binding domain is a scFv that comprises a sequence at least 90%, at least 95%, or 100% identical to SEQ ID NO: 27.

78. The method of claim 69, wherein each polypeptide comprises a sequence at least 90%, at least 95%, or 100% identical to SEQ ID NO: 31.

79. The method of any one of claims 68-78, wherein the multispecific protein is administered to the subject by IV infusion.

80. The method of any one of claims 68-79, wherein the multispecific protein is administered the subject by IV infusion at a dose of 0.1, 0.2, 0.3, 0.4, 0.5, 0.6, 0.7, or 0.8 μg/kg.

81. The method of any one of claims 68-79, wherein the multispecific protein is administered the subject by IV infusion at a dose of 0.3, 1, 3, 6, 9, 12, 18, 20, 24, 30, 36, 48, 50, 60, 75, or 100 μg.

82. The method of any one of claims 68-81, wherein the multispecific protein is administered once per week.

83. The method of claim 82, wherein the multispecific protein is administered to the subject by IV infusion during a first 28-day cycle; wherein 6 μg of the multispecific protein is administered on day 8; 12 μg of the multispecific protein is administered on day 15; and 18 μg of the multispecific protein is administered on day 22 of the first 28-day cycle.

84. The method of claim 83, wherein the multispecific protein is administered to the subject by IV infusion during at least one additional 28-day cycle following the first 28-day cycle, wherein 18 μg of the multispecific protein is administered on days 1, 8, 15, and 22 of the at least one additional 28-day cycle.

85. The method of claim 82, wherein the multispecific protein is administered to the subject by IV infusion during a first 28-day cycle; wherein 6 μg of the multispecific protein is administered on day 15; and 12 μg of the multispecific protein is administered on day 22 of the first 28-day cycle.

86. The method of claim 85, wherein the multispecific protein is administered to the subject by IV infusion during at least one additional 28-day cycle following the first 28-day cycle, wherein 18 μg of the multispecific protein is administered on days 1, 8, 15, and 22 of the at least one additional 28-day cycle.

87. The method of claim 82, wherein the multispecific protein is administered to the subject by IV infusion during a first 28-day cycle, wherein 6 μg of the multispecific protein is administered on day 1; 8 μg of the multispecific protein is administered on day 12; 18 μg of the multispecific protein is administered on day 15; and 18 μg of the multispecific protein is administered on day 22 of the first 28-day cycle.

88. The method of claim 87, wherein the multispecific protein is administered to the subject by IV infusion during at least one additional 28-day cycle following the first 28-day cycle, wherein 18 μg of the multispecific protein is administered on days 1, 8, 15, and 22.

89. The method of claim 82, wherein the multispecific protein is administered to the subject by IV infusion during a first 28-day cycle, wherein 6 μg of the multispecific protein is administered on day 1; 8 μg of the multispecific protein is administered on day 12; 18 μg of the multispecific protein is administered on day 15; and 18 μg of the multispecific protein is administered on day 22 of the first 28-day cycle.

90. The method of claim 89, wherein the multispecific protein is administered to the subject by IV infusion during at least one additional 28-day cycle following the first 28-day cycle, wherein 18 μg of the multispecific protein is administered on days 1, 8, 15, and 22 of the at least one additional 28-day cycle.

91. The method of any one of claims 68-90, wherein the cancer is a carcinoma or sarcoma.

92. The method of any one of claims 68-90, wherein the cancer is melanoma, kidney cancer, pancreatic cancer, lung cancer, intestinal cancer, prostate cancer, breast cancer, liver cancer, brain cancer, colon cancer, ovarian cancer, or hematological cancer.

93. The method of any one of claims 68-90, wherein the cancer is acute myeloid leukemia (AML), myelodysplastic syndrome (MDS), hairy cell leukemia (HCL), blastic plasmacytoid dendritic cell neoplasm, B-cell acute lymphoblastic leukemia (ALL), or chronic myeloid leukemia (CML).

94. The method of any one of claims 68-90, wherein the cancer is acute myeloid leukemia (AML).

95. The method of any one of claims 68-90, wherein the cancer is myelodysplastic syndrome (MDS).
